(19) United States
(12) Patent Application Publication
(10) Pub. No.: US 2017/0305893 A1
(43) Pub. Date: Oct. 26, 2017

(54) HETEROCYCLIC COMPOUNDS AS DCTPP1 MODULATORS

(71) Applicant: Thomas Helledays Stiftelse for Medicinsk Forskning, Stocksund (SE)

(72) Inventors: Sabin LLONA-MINGUEZ, Solna (SE); Andreas HOGLUND, Stockholm (SE); Sylvain JACQUES, Lyon (FR); Lars JOHANSSON, Bromma (SE); Tobias KOOLMEISTER, Stockholm (SE); Martin SCOBIE, Uppsala (SE); Thomas HELLEDAY, Stocksund (SE)

(21) Appl. No.: 15/517,530

(22) PCT Filed: Oct. 8, 2015
(86) PCT No.: PCT/EP2015/073289

§ 371 Date: Apr. 7, 2017

Related U.S. Application Data
(60) Provisional application No. 62/061,377, filed on Oct. 8, 2014.

(51) Int. Cl. C07D 417/10; C07F 5/02; C07D 405/04; C07D 403/10; C07D 401/10; C07D 235/12; A61K 31/4184; C07D 235/08; A61K 45/06; A61K 31/69; A61K 31/4439; A61K 31/427; A61K 31/4196; C07D 235/10; C07F 5/05

(52) U.S. Cl. CPC C07D 417/10; C07F 5/05; C07F 5/025; C07D 405/04; C07D 403/10; C07D 401/10; C07D 235/12; A61K 31/4184; C07D 235/08; A61K 45/06; A61K 31/69; A61K 31/4439; A61K 31/427; A61K 31/4196; C07D 235/10

(57) ABSTRACT

The invention relates to compounds of formula I, or a pharmaceutically-acceptable salt thereof. The present invention also relates to pharmaceutical formulations comprising these compounds, and to their use as medicaments for the treatment of disorders where modulation of DCTPP (deoxycytidine triphosphate pyrophosphatase 1) activity exerts a therapeutic effect.

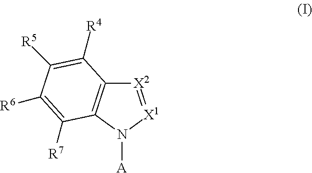

### FIELD OF THE INVENTION

The invention relates to novel compounds and pharmaceutically acceptable salts thereof. The present invention also relates to pharmaceutical formulations comprising these compounds, and to their use as medicaments for the treatment of disorders where modulation of DCTPP1 (deoxycytidine triphosphate pyrophosphatase 1) activity exerts a therapeutic effect.

### BACKGROUND OF THE INVENTION

The listing or discussion of an apparently prior-published document in this specification should not necessarily be taken as an acknowledgement that the document is part of the state of the art or is common general knowledge.

Non-canonical nucleotides are by-products of cellular metabolism. Incorporation of these damaged nucleotides during DNA replication results in mispairing, mutations and cell death. Cells have developed mechanisms to maintain the integrity of the nucleotide pool and minimize misincorporation of non-canonical nucleotides. Nucleoside triphosphate pyrophosphatases are “housecleaning” enzymes that hydrolyse non-canonical tri-phosphates to the corresponding mono- or di-phosphates. Due to its role in nucleic acid metabolism this class of enzymes can regulate cell proliferation and survival.

The dCTP pyrophosphatase enzyme (DCTPP1) is highly expressed in cancer tissue and tumour cell lines (Eur J Histochem. 2013 57(3):e29). DCTPP1 efficiently hydrolyses non-canonical nucleotides of biological relevance, including 5-Me-dCTP, 5-formyl-dCTP and 5-halo-dCTPs (Biochem J. 2014; 459(1):171-80). DCTPP1-depleted cells show increased concentration of intracellular dCTP and become more sensitive to cytotoxic nucleoside derivatives.

Synthetic nucleoside analogues have been employed in the treatment of cancer. These nucleoside analogues may be phosphorylated in the intracellular environment and then incorporated into DNA, where they exert their biological action through different mechanisms, such as inhibition of DNA methyltransferases or DNA polymerases. Due to their structural similarity with canonical and non-canonical nucleotides, phosphorylated nucleoside analogues can serve as substrates of pyrophosphatase enzymes, such as DCTPP1, and hence become inactive. Hence, modulators of the DCTPP1 enzyme can be useful in the treatment or prevention of proliferative disorders such as various forms of cancer, used alone or in combination with nucleoside analogues.

During chronic inflammation, cells from the immune system such as eosinophils, neutrophils and monocytes, secrete enzymes capable of generating mutagenic 5-halo-dCTPs (J Biol Chem. 1999 (47):33440-8; Proc Natl Acad Sci USA. 2001; 98(4):1631-6; Biochimica et Biophysica Acta, 525 (1978) 37-44). Locally generated halogenated products can be incorporated in cellular and/or mitochondrial DNA. Inhibition of DCTPP1 could lead to decreased degradation of 5-halo-dCTPs and subsequent increased intracellular levels, affecting repair and synthesis of genomic material and protein synthesis in immune system cells (Int Arch Allergy Immunol. 2010; 152(1):12-2; Haematologica. 2007 October; 92(10):1311-8). Hence, modulators of the DCTPP1 enzyme can be useful in the treatment or prevention of inflammatory or allergic conditions.

Today's treatment of cancer is not effective for all patients with a diagnosed disorder, also including a large proportion of patients that experience adverse effects from treatments with existing therapies or where resistance to on-going therapy is developed over time.

There are many disorders that are inflammatory in their nature or have an inflammatory component. One of the major problems associated with existing treatments of inflammatory conditions is a lack of efficacy and/or the prevalence of side effects (real or perceived).

Asthma is a chronic inflammatory disorder affecting 6% to 8% of the adult population of the industrialized world. In children, the incidence is even higher, being close to 10% in most countries. Asthma is the most common cause of hospitalization for children under the age of fifteen. Treatment regimens for asthma are based on the severity of the condition. Mild cases are either untreated or are only treated with inhaled β-agonists. Patients with more severe asthma are typically treated with anti-inflammatory compounds on a regular basis. There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy. Indeed, non-allergic conditions of this class are in many cases more difficult to treat.

Chronic obstructive pulmonary disorder (COPD) is a common disorder affecting 6% to 8% of the world population. The disorder is potentially lethal, and the morbidity and mortality from the condition is considerable. At present, there is no known pharmacological treatment capable of changing the course of COPD.

Other inflammatory disorders which may be mentioned include:
(a) pulmonary fibrosis (this is less common than COPD, but is a serious disorder with a very bad prognosis. No curative treatment exists);
(b) inflammatory bowel disorder (a group of disorders with a high morbidity rate. Today only symptomatic treatment of such disorders is available);
(c) rheumatoid arthritis and osteoarthritis (common disabling inflammatory disorders of the joints. There are currently no curative, and only moderately effective symptomatic, treatments available for the management of such conditions);
(d) Hypereosininophilic syndromes; and
(e) Allergy disorders.

Inflammation is also a common cause of pain. Inflammatory pain may arise for numerous reasons, such as infection, surgery or other trauma. Moreover, several malignancies are known to have inflammatory components adding to the symptomatology of the patients.

Kambe, T et al. Journal of the American Chemical Society (2014), 136, 10777-10782, discuss probes targeting various proteins, e.g. DCTPP1. However, the compounds do not show any structural resemblance to the compounds of this invention and they are not suggested to be of pharmaceutical use.

Corson, T W et al. ChemBioChem (2011), 12(11), 1767-1773, disclose the natural product triptolide as a DCTPP1 inhibitor. However, triptolide has no structural relationship to the compounds of this invention.

WO 2014/096388 describes certain benzimidazoles as kinase inhibitors. When the benzimidazole is substituted in the 4-position with a nitro group, the 2-substituent is either bromo, aminoalkyl, heterocycloalkyl, or cycloalkyl substituted with an amino group. Moreover, the benzimidazole 1-substituent cannot be aromatic or contain a group which carries an aromatic substituent.

WO 2010/118155 and WO 2008/063300 describe the use of certain heteroarylboronates as inhibitors of fatty acid amide hydrolase, but there is nothing that suggests that the compounds are useful in the treatment of cancer.

Liou, J-P et al. Journal of Medicinal Chemistry (2008), 51, 4351-4355, evaluates certain compounds for their antiproliferative activities against three types of human cancer cell lines, e.g. (5-methoxy-4-nitroindol-1-yl)(3,4,5-trimethoxy-phenyl)methanone. However, this compound does not show any activity.

WO 2008/068171 describes certain pyrimidine derivatives as JNK modulators useful for various disorders including cancer.

WO 2007/134169 and WO 2006/050053 disclose benzimidazole, indole and benzolactam boronic acid compounds as inhibitors of TNF-α. The compounds are described as anti-inflammatory agents but are also suggested to be useful in the treatment of cancer. However, there is no data in the documents that supports such a suggestion. In addition, the boronic acid/ester moiety are in all exemplified cases linked to the heterocyclic part of the molecule via an aliphatic linker that does not contain an aromatic ring. Also, benzimidazoles or indoles carrying a nitro or a carbonyl functionality in their 4-positions have not been prepared and no biological results are available.

WO 2006/033620 and WO 2004/100865 describe the use of certain 1-substituted indoles and benzimidazoles in the treatment of various pain disorders. The compounds are also claimed to be useful in the treatment of cancer, but there is no evidence that supports such a claim. In addition, the 1-substituent contains an alkylcarboxamide linker.

WO 2006/071609 describes glucocorticoid mimetic ligands having anti-inflammatory and immune suppressive activities. Although a method of treatment of tumor disorders is claimed, there is nothing that supports that speculative claim.

EP 563001 discloses the use of (4-(5-trifluoromethylbenzimidazol-1-yl)phenyl)boronic acid as an intermediate in the synthesis of compounds that blocks L-type calcium channels and which compounds are useful in the treatment of certain CNS disorders. There is nothing that suggests that this compound can be used in the treatment of proliferative disorders.

### SUMMARY OF THE INVENTION

Although the finding of oncogenes and development of new anticancer treatments and diagnosis have improved the life length of cancer patients, there is still a high medical need to find more effective and less toxic treatments. DCTPP1 inhibitors have the potential to have improved efficacy against proliferative disorders such as inflammation and/or cancer forms with dysfunctional DCTPP1 status, with decreased general toxic effects compared to known compounds. DCTPP1 inhibition may also be a suitable adjuvant therapy to be used in conjunction with radiotherapies or other chemotherapeutic approaches.

There is a real and substantial unmet clinical need for an effective anti-inflammatory drug capable of treating inflammatory disorders, in particular asthma and COPD, with no real or perceived side effects.

In a first aspect of the invention, there is provided a compound of formula I,

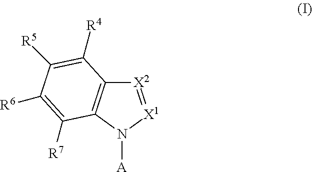

or a pharmaceutically acceptable salt thereof,
for use in the treatment of proliferative disorders, such as cancer and inflammation wherein:
A represents -L¹-L²-L³-A¹;
A¹ represents aryl optionally substituted by one or more Y¹, or heteroaryl optionally substituted by one or more Y²;
each one of L¹ and L³ independently represents a single bond or C₁₋₃alkylene optionally substituted by one or more halo;
L² represents a single bond, —C(Q)-, —N(R¹)—, —O— or —S(O)ₙ—;
X¹ represents C(R²) or N;
X² represents C(R³) or N;
each R¹ and R³ independently represents H or C₁₋₆alkyl optionally substituted by one or more halo;
R² represents H, R^{a} or —OR^{b};
R⁴ and R⁷ independently represent H, halo, —CN, R^{c}, —N₃, —NO₂, —N(R^{d})R^{e}, —N(R^{f})C(Q¹)R^{g}, —N(R^{h})S(O)ₙRⁱ, —OR^{j} or —SR^{k};
R⁵ and R⁶ independently represent H, halo, —CN, R^{c}, —N₃ or —NO₂; or
R⁴ and R⁵, R⁵ and R⁶ and/or R⁶ and R⁷ are linked together to form, along with the carbon atoms to which they are attached, a 5- or 6-membered ring, which ring optionally contains one to three heteroatoms and/or one or two further double bonds, and which ring optionally is substituted by one or more groups independently selected from halo, —OR^{j}, C₁₋₃alkyl optionally substituted by one or more halo, and Q¹;
Q represents ═O or ═S;
Q¹ represents ═O, ═NR^{r} or ═S;
R^{a} represents C₁₋₆alkyl optionally substituted by one or more groups independently selected from D¹, aryl optionally substituted by one or more groups independently selected from D² or heteroaryl optionally substituted by one or more groups independently selected from D³;
each R^{c} independently represents C₁₋₆alkyl optionally substituted by one or more halo;
each R^{b}, R^{d}, R^{e}, R^{f}, R^{g}, R^{h} and Rⁱ independently represents H or C₁₋₆alkyl optionally substituted by one or more halo; or
R^{d} and R^{e} are linked together to form, along with the nitrogen atom to which they are attached, a 3- to 6-membered ring, which ring optionally contains one further heteroatom and which ring optionally is substituted by one or more halo, one or more C₁₋₃alkyl each optionally and independently substituted by one or more F, or ═O;
D¹ represents halo, —OC₁₋₆alkyl optionally substituted by one or more halo, aryl optionally substituted by one or more groups independently selected from D² or heteroaryl optionally substituted by one or more groups independently selected from D³;
each D² and D³ independently represents halo, C₁₋₆alkyl optionally substituted by one or more halo or —OC₁₋₆alkyl optionally substituted by one or more halo; each Y¹ and Y² independently represents halo, —BF₃M, —B(OR^{a1})₂, R^{b1}, —CN, —C(Q²)R^{c1}, —C(Q²)OR^{d1}, —C(Q²)N(R^{e1})R^{f1}, —N₃, —NO₂, —N(R^{g1})R^{h1}, —N(Rⁱ¹)C(Q²)R^{j1}, —N(R^{k1})C(Q²)N(R^{l1})R^{m1}, —N(Rⁿ¹)C(Q²)OR^{o1}, —N(R^{p1})S(O)ₙR^{q1}, —N(R^{r1})S(O)ₙN(R^{s1})R^{t1}, —OR^{u1}, —OC(Q²)R^{v1}, —OC(Q²)N(R^{w1})R^{x1}, —OC(Q²)OR^{y1}, —OS(O)ₙR^{z1}, —SR^{aa1}, —S(Q)ₙR^{ab1}, —S(O)ₙN(R^{ac1})R^{ad1}, heterocycloalkyl optionally substituted by one or more groups independently selected from Z¹, aryl substituted by one or more groups independently selected from Z², heteroaryl optionally substituted by one or more groups independently selected from Z³ or Q²;
each Z¹ independently represents halo, R^{b1}, —CN, —C(Q²)R^{c1}, —C(Q²)OR^{d1}, —C(Q²)N(R^{e1})R^{f1}, —N₃, —NO₂, —N(R^{g1})R^{h1}, —N(Rⁱ¹)C(Q²)R^{j1}, —N(R^{k1})C(Q²)N(R^{l1})R^{m1}, —N(Rⁿ¹)C(Q²)OR^{o1}, —N(R^{p1})S(O)ₙR^{q1}, —N(R^{r1})S(O)ₙN(R^{s1})R^{t1}, —OR^{u1}, —OC(Q²)R^{v1}, —OC(Q²)N(R^{w1})R^{x1}, —OC(Q²)OR^{y1}, —OS(O)ₙR^{z1}, —SR^{aa1}, —S(O)ₙR^{ab1}, —S(O)ₙN(R^{ac1})R^{ad1} or Q²;
each Z² and Z³ independently represents halo, —BF₃M, —B(OR^{a1})₂, R^{b1}, —CN, C(Q²)R^{c1}, —C(Q²)OR^{d1}, —C(Q²)N(R^{e1})R^{f1}, —N₃, —NO₂, —N(R^{g1})R^{h1}, —N(Rⁱ¹)C(Q²)R^{j1}, —N(R^{k1})C(Q²)N(R^{l1})R^{m1}, —N(Rⁿ¹)C(Q²)OR^{o1}, —N(R^{p1})S(O)ₙR^{q1}, —N(R^{r1})S(O)ₙN(R^{s1})R^{t1}, —OR^{u1}, —OC(Q²)R^{v1}, —OC(Q²)N(R^{w1})R^{x1}, —OC(Q²)OR^{y1}, —OS(O)ₙR^{z1}, —SR^{aa1}, —S(O)ₙR^{ab1} or —S(O)ₙN(R^{ac1})R^{ad1};
M represents a cation selected from (R^{M})₄N⁺, Li⁺, Na⁺, K⁺, Rb⁺or Cs⁺;
each R^{M} independently represents C₁₋₁₂alkyl optionally substituted by one or more D⁴;
each Q² independently represents ═NR^{ae1}, ═N(OR^{af1}), ═O or ═S;
each R^{b1}, R^{o1}, R^{q1}, R^{y1}, R^{z1} and R^{ab1} independently represents C₁₋₄ alkyl optionally substituted by one or more groups independently selected from D⁴;
each R^{a1}, R^{c1}, R^{d1}, R^{e1}, R^{f1}, R^{g1}, R^{h1}, Rⁱ¹, R^{j1}, R^{k1}, R^{l1}, R^{m1}, Rⁿ¹, R^{p1}, R^{r1}, R^{s1}, R^{t1}, R^{u1}, R^{v1}, R^{w1}, R^{x1}, R^{aa1}, R^{ac1}, R^{ad1}, R^{ae1}, and R^{af1} independently represents H or C₁₋₄alkyl optionally substituted by one or more groups independently selected from D⁴; or
R^{e1} and R^{f1}, R^{g1} and R^{h1}, R^{l1} and R^{m1}, R^{s1} and R^{t1}, R^{w1} and R^{x1} and/or R^{ac1} and R^{ad1} are linked together to form, along with the nitrogen atom to which they are attached, a 3- to 6-membered ring, which ring optionally contains one further heteroatom and which ring optionally is substituted by one or more groups independently selected from F, one or more C₁₋₃alkyl each optionally and independently substituted by one or more F, or ═O; or
two R^{a1} are linked together to form, along with the boron, and the oxygen atoms to which they are attached, a 5- to 8-membered heterocyclic ring, which ring optionally contains one or more further heteroatoms and which ring optionally is substituted by one or more groups independently selected from halo, one or more C₁₋₃alkyl optionally substituted by one or more halo, and/or one or more ═O;
each D⁴ independently represents halo, —OH or —OC₁₋₆alkyl optionally substituted by one or more halo; and
each n independently represents 1 or 2;
provided that at least one of R⁴ or R⁷ represents —NO₂.

One embodiment of the first aspect of the invention relates to compounds of formula I, wherein:

A represents -L¹-L²-L³-A¹;
A¹ represents aryl optionally substituted by one or more groups independently selected from Y¹ or heteroaryl optionally substituted by one or more groups independently selected from Y²;
each one of L¹ and L³ independently represents a single bond or C₁₋₃alkylene;
L² represents a single bond, —C(Q)-, or —S(O)ₙ—;
X¹ represents C(R²) or N;
X² represents C(R³) or N;
R³ represents H;
R² represents H, R^{a} or —OR^{b};
R⁴ and R⁷ independently represent H or —NO₂;
R⁵ and R⁶ independently represent H, halo or R^{c}; or
R⁵ and R⁶ are linked together to form, along with the carbon atoms to which they are attached, a 5- or 6-membered ring, which ring optionally contains one to three heteroatoms and/or one or two further double bonds, and which ring optionally is substituted by C₁₋₃alkyl;
Q represents ═O;
R^{a} represents C₁₋₆alkyl optionally substituted by one or more groups independently selected from D¹ or aryl optionally substituted by one or more groups independently selected from D²;
each R^{c} independently represents C₁₋₆alkyl optionally substituted by one or more halo;
each R^{b} represents H or C₁₋₆alkyl;
D¹ represents halo, —OC₁₋₆alkyl optionally substituted by one or more aryl; each Y¹ and Y² independently represents halo, —BF₃M, —B(OR^{a1})₂, R^{b1}, —CN, —C(Q²)R^{c1}, —C(Q²)OR^{d1}, —C(Q²)N(R^{e1})R^{f1}, —NO₂, —N(R^{g1})R^{h1}, —N(Rⁱ¹)C(Q²)R^{j1}, —OR^{u1}, —OC(Q²)R^{v1}, —S(O)ₙR^{ab1} or heteroaryl;
M represents a cation selected from (R^{M})₄N⁺, Li⁺, Na⁺, K⁺, Rb⁺ or Cs⁺;
each R^{M} independently represents C₁₋₁₂alkyl optionally substituted by one or more D⁴;
each R^{b1} and R^{ab1} independently represents C₁₋₄ alkyl;
each R^{a1}, R^{c1}, R^{g1}, R^{h1}, Rⁱ¹, R^{j1}, R^{u1} and R^{v1} independently represents H or C₁₋₄ alkyl; or two R^{a1} are linked together to form, along with the boron, and the oxygen atoms to which they are attached, a 5- to 8-membered heterocyclic ring, which ring optionally contains one or more further heteroatoms and which ring optionally is substituted by one or more groups independently selected from halo, one or more C₁₋₃alkyl optionally substituted by one or more halo, and/or one or more ═O;
each D⁴ independently represents halo, —OH or —OC₁₋₆alkyl optionally substituted by one or more halo;
each n independently represents 1 or 2; and
provided that at least one of R⁴ or R⁷ represents —NO₂.

In a second aspect of the invention there is provided a novel compound of formula I,

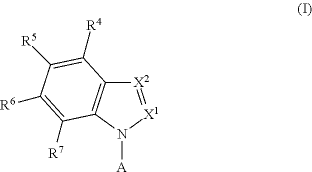

or a pharmaceutically acceptable salt thereof,
wherein:
A represents -L¹-L²-L³-A¹;
A¹ represents:
(i) aryl substituted by —BF₃M or —B(OR^{a1})₂, and optionally substituted by one or more groups independently selected from Y¹;
(ii) heteroaryl substituted by —BF₃M or —B(OR^{a1})₂, and optionally substituted by one or more groups independently selected from Y²; or
(iii) bicyclic, boron containing, partly aromatic heteroaryl substituted on the boron by —OH and optionally substituted by one or more groups independently selected from Y³;
   each one of L¹ and L³ independently represents a single bond or C₁₋₃alkylene optionally substituted by one or more halo;
   L² represents a single bond, —C(Q)-, —N(R¹)—, —O—, —S(O)ₙ—, —C(Q)N(R¹)—, —N(R¹)C(Q)-, —C(O)O—, —OC(O)—, —S(O)ₙN(R¹)— or —N(R¹)S(O)ₙ—;
   X¹ represents C(R²);
   X² represents N;
   each R¹ independently represents H or C₁₋₆alkyl optionally substituted by one or more halo;
   R² represents H, R^{a} or —OR^{b};
   R⁴ and R⁷ independently represent H, halo, —CN, R^{c}, —C(H)(CF₃)OH, —C(CF₃)₂OH, —C(OH)₂CF₃, —N₃, —NO₂, —N(R^{d})R^{e}, —N(R^{f})C(Q¹)R^{g}, —N(R^{h})S(O)ₙRⁱ, —OR^{j}, —SR^{k} or —C(O)R⁸;
   R⁵ and R⁶ independently represent H, halo, —CN, R^{c}, —N₃, —NO₂, —OR^{j} or —SR^{k}; or
   R⁴ and R⁵, R⁵ and R⁶ and/or R⁶ and R⁷ are linked together to form, along with the carbon atoms to which they are attached, a 5- or 6-membered ring, which ring optionally contains one to three heteroatoms and/or one or two further double bonds, and which ring optionally is substituted by one or more groups independently selected from halo, —OR^{j}, C₁₋₃alkyl optionally substituted by one or more halo, and Q¹;
   each R⁸ independently represents —OR^{l}, —N(H)R^{m}, —N(H)C(Q¹)Rⁿ, —N(H)C(Q¹)N(R^{o})R^{p}, —N(H)OH or —N(H)S(O)ₙR^{q};
   Q represents ═O or ═S;
   Q¹ represents ═O, ═NR^{r} or ═S;
   R^{a} represents C₁₋₆alkyl optionally substituted by one or more groups independently selected from D¹, aryl optionally substituted by one or more groups independently selected from D² or heteroaryl optionally substituted by one or more groups independently selected from D³;
   each R^{c} and R^{q} independently represents C₁₋₆alkyl optionally substituted by one or more halo;
   each R^{b}, R^{d}, R^{e}, R^{f}, R^{g}, R^{h}, Rⁱ, R^{j}, R^{k}, R^{l}, R^{m}, Rⁿ, R^{o}, R^{p} and R^{r} independently represents H or C₁₋₆alkyl optionally substituted by one or more halo; or
   R^{d} and R^{e} and/or R^{o} and R^{p} are linked together to form, along with the nitrogen atom to which they are attached, a 3- to 6-membered ring, which ring optionally contains one further heteroatom and which ring optionally is substituted by one or more halo, one or more C₁₋₃alkyl each optionally and independently substituted by one or more F, or ═O;
   D¹ represents halo, —OC₁₋₆alkyl optionally substituted by one or more halo, aryl optionally substituted by one or more groups independently selected from D² or heteroaryl optionally substituted by one or more groups independently selected from D³;
   each D² and D³ independently represents halo, C₁₋₆alkyl optionally substituted by one or more halo or —OC₁₋₆alkyl optionally substituted by one or more halo;
   each Y¹, Y² and Y³ independently represents halo, R^{b1}, —CN, or —OR^{u1};
   M represents a cation selected from (R^{M})₄N⁺, Li⁺, Na⁺, K⁺, Rb⁺ or Cs⁺;
   each R^{M} independently represents C₁₋₁₂alkyl optionally substituted by one or more D⁴;
   each R^{b1} independently represents C₁₋₆ alkyl optionally substituted by one or more groups independently selected from D⁴;
   each R^{a1} and R^{u1} independently represents H or C₁₋₆ alkyl optionally substituted by one or more groups independently selected from D⁴; or
   two R^{a1} are linked together to form, along with the boron, and the oxygen atoms to which they are attached, a 5- to 8-membered heterocyclic ring, which ring optionally contains one or more further heteroatoms and which ring optionally and independently is substituted by one or more groups independently selected from halo, C₁₋₃alkyl optionally substituted by one or more halo, and ═O;
   each D⁴ independently represents halo, —OH or —OC₁₋₆alkyl optionally substituted by one or more halo;
   each n independently represents 1 or 2;
   provided that at least one of R⁴ and R⁷ represents —C(H)(CF₃)OH, —C(CF₃)₂OH, —C(OH)₂CF₃, —NO₂ or —C(O)R⁸; and
   provided that formula I does not represent
1-(4-boronobenzyl)-2-ethoxy-1H-benzo[d]imidazole-7-carboxylic acid,
ethyl 1-(4-(5,5-dimethyl-1,3,2-dioxaborinan-2-yl)benzyl)-2-ethoxy-1H-benzo[d]imidazole-7-carboxylate,
methyl 1-(3-(4,4,5,5-tetramethyl-1,3,2-dioxaborolan-2-yl)benzyl)-1H-indole-7-carboxylate, or
methyl 1-(4-(4,4,5,5-tetramethyl-1,3,2-dioxaborolan-2-yl)benzyl)-1H-indole-7-carboxylate.

One embodiment of the second aspect of the invention relates to compounds of formula I, wherein:

A represents -L¹-L²-L³-A¹;
A¹ represents:
(i) aryl substituted by —BF₃M or —B(OR^{a1})₂, and optionally substituted by one or more groups independently selected from Y¹;
(ii) heteroaryl substituted by —BF₃M or —B(OR^{a1})₂, and optionally substituted by one or more groups independently selected from Y²; or
   each one of L¹ and L³ independently represents a single bond or C₁₋₃alkylene;
   L² represents a single bond, —C(Q)-, or —S(O)ₙ—;
   X¹ represents C(R²);
   X² represents N;
   R² represents H, R^{a} or —OR^{b};
   R⁴ and R⁷ independently represent H or —NO₂;
   R⁵ and R⁶ independently represent H, halo or R^{c}; or
   R⁵ and R⁶ are linked together to form, along with the carbon atoms to which they are attached, a 5- or 6-membered ring, which ring optionally contains one to three heteroatoms and/or one or two further double bonds, and which ring optionally is substituted by C₁₋₃alkyl;
   Q represents ═O;
   R^{a} represents C₁₋₆alkyl optionally substituted by one or more groups independently selected from D¹, aryl optionally substituted by one or more groups independently selected from D²;
   each R^{b}, represents H or C₁₋₆alkyl;
   each R^{c} and R^{q} independently represents C₁₋₆alkyl optionally substituted by one or more halo;
   D¹ represents halo, —OC₁₋₆alkyl optionally substituted by one or more aryl; each Y¹ and Y² independently represents halo, —BF₃M, —B(OR^{a1})₂, R^{b1}, —CN, —C(Q²)R^{c1}, —C(Q²)OR^{d1}, —C(Q²)N(R^{e1})R^{f1}, —NO₂, —N(R^{g1})R^{h1}, —N(Rⁱ¹)C(Q²)R^{j1}, —OR^{u1}, —OC(Q²)R^{v1}, —S(O)ₙR^{ab1}, heteroaryl;
   M represents a cation selected from (R^{M})₄N⁺, Li⁺, Na⁺, K⁺, Rb⁺ or Cs⁺;
   each R^{M} independently represents C₁₋₁₂alkyl optionally substituted by one or more D⁴;
   each R^{b1} and R^{ab1} independently represents C₁₋₄ alkyl;
   each R^{a1}, R^{c1}, R^{g1}, R^{h1}, Rⁱ¹, R^{j1}, R^{u1}, R^{v1}, independently represents H or C₁₋₄ alkyl;
   or two R^{a1} are linked together to form, along with the boron, and the oxygen atoms to which they are attached, a 5- to 8-membered heterocyclic ring, which ring optionally contains one or more further heteroatoms and which ring optionally is substituted by one or more groups independently selected from halo, one or more C₁₋₃alkyl optionally substituted by one or more halo, and/or one or more ═O;
   each D⁴ independently represents halo, —OH or —OC₁₋₆alkyl optionally substituted by one or more halo;
   each n independently represents 1 or 2; and
   provided that formula I does not represent
1-(4-boronobenzyl)-2-ethoxy-1H-benzo[d]imidazole-7-carboxylic acid, ethyl 1-(4-(5,5-dimethyl-1,3,2-dioxaborinan-2-yl)benzyl)-2-ethoxy-1H-benzo[d]imidazole-7-carboxylate,
methyl 1-(3-(4,4,5,5-tetramethyl-1,3,2-dioxaborolan-2-yl)benzyl)-1H-indole-7-carboxylate, or
methyl 1-(4-(4,4,5,5-tetramethyl-1,3,2-dioxaborolan-2-yl)benzyl)-1H-indole-7-carboxylate.

Another embodiment of the second aspect of the invention relates to compounds of formula I, wherein:

A represents -L¹-L²-L³-A¹;
A¹ represents:
(i) aryl substituted by —BF₃M or —B(OR^{a1})₂, and optionally substituted by one or more groups independently selected from Y¹;
(ii) heteroaryl substituted by —BF₃M or —B(OR^{a1})₂, and optionally substituted by one or more groups independently selected from Y²; or
   each one of L¹ and L³ independently represents a single bond or C₁₋₃alkylene;
   L² represents a single bond, —C(Q)-, or —S(O)ₙ—;
   X¹ represents C(R²);
   X² represents N;
   R² represents R^{a};
   R⁴ and R⁷ independently represent H or —NO₂;
   R⁵ and R⁶ independently represent H, F, Cl or methyl; or
   R⁵ and R⁶ are linked together to form, along with the carbon atoms to which they are attached, a 5- or 6-membered ring, which ring optionally contains one to three heteroatoms and/or one or two further double bonds, and which ring optionally is substituted by C₁₋₃alkyl;
   Q represents ═O;
   R^{a} represents C₁₋₆alkyl;
   each R^{b}, represents H or C₁₋₆alkyl;
   M represents a cation selected from (R^{M})₄N⁺, Li⁺, Na⁺, K⁺, Rb⁺ or Cs⁺;
   each R^{M} independently represents C₁₋₁₂alkyl optionally substituted by one or more D⁴;
   each R^{b1} and R^{ab1} independently represents C₁₋₄ alkyl;
   each R^{a1}, R^{c1}, R^{g1}, R^{h1}, Rⁱ¹, R^{j1}, R^{u1}, R^{v1} independently represents H or C₁₋₄alkyl; or
   two R^{a1} are linked together to form, along with the boron, and the oxygen atoms to which they are attached, a 5- to 8-membered heterocyclic ring, which ring optionally contains one or more further heteroatoms and which ring optionally is substituted by one or more groups independently selected from halo, one or more C₁₋₃alkyl optionally substituted by one or more halo, and/or one or more ═O;
   each D⁴ independently represents halo, —OH or —OC₁₋₆alkyl optionally substituted by one or more halo;
   each n independently represents 1 or 2; and
   provided that formula I does not represent
1-(4-boronobenzyl)-2-ethoxy-1H-benzo[d]imidazole-7-carboxylic acid, ethyl 1-(4-(5,5-dimethyl-1,3,2-dioxaborinan-2-yl)benzyl)-2-ethoxy-1H-benzo[d]imidazole-7-carboxylate,
methyl 1-(3-(4,4,5,5-tetramethyl-1,3,2-dioxaborolan-2-yl)benzyl)-1H-indole-7-carboxylate, or
methyl 1-(4-(4,4,5,5-tetramethyl-1,3,2-dioxaborolan-2-yl)benzyl)-1H-indole-7-carboxylate.

Compounds of the invention may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. In particular, compounds of the invention may have the advantage that they are more efficacious and/or exhibit advantageous properties in vivo.

Compounds of the invention may make anti-proliferative agents (such as e.g. anti-cancer and/or anti-inflammatory agents) with which they are combined more efficacious (i.e. allowing the effective dose of the anti-proliferative agent to be decreased and thus lower the risk of adverse reaction), prolong the duration of the effect of anti-proliferative agents with which they are combined and/or decrease the risk of resistance to the anti-proliferative agents with which they are combined.

The invention further relates to a pharmaceutical formulation comprising a compound of the invention according to the first or second aspect in admixture with one or more pharmaceutically-acceptable adjuvant, diluent and/or carrier.

The invention also relates to a combination product comprising a compound of the invention according to the first or second aspect together with one or more therapeutically agent and a kit-of-part comprising said combination product.

The invention further relates to compounds of the invention according to the first and second aspect, a pharmaceutical formulation comprising said compounds, or a combination product or kit-of-part as mentioned above, for use in therapy, such as in the treatment of proliferative disorders, e.g. cancer and/or inflammation.

The invention relates to the compounds of the invention according to the first and second aspect, a pharmaceutical formulation comprising said compounds, or a combination product or kit-of-part as mentioned above, for use in therapy, or use in the treatment of conditions associated with modulation of DCTPP1 (deoxycytidine triphosphate pyrophosphatase 1) activity, such as in the treatment of proliferative disorders, e.g. cancer and/or inflammation.

The invention also relates to a method of treatment, of a condition associated with modulation of DCTPP1 (deoxycytidine triphosphate pyrophosphatase 1) activity, such as in the treatment of proliferative disorders, e.g. cancer and/or inflammation, comprising administrating to a mammal, including human, in need of such treatment a therapeutically effective amount of the compound of the invention according to the first and second aspect, a pharmaceutical formulation comprising said compounds, or a combination product or kit-of-part as mentioned above.

The invention further relates to a use of the compounds of the invention according to the first and second aspect, a pharmaceutical formulation comprising said compounds, or a combination product or kit-of-part as mentioned above, in the manufacturing of a medicament for the treatment of conditions associated with modulation of DCTPP1 (deoxycytidine triphosphate pyrophosphatase 1) activity, such as in the treatment of proliferative disorders, e.g. cancer and/or inflammation.

### BRIEF DESCRIPTION OF THE DRAWINGS

FIG. 1. Graph showing combination effects of compound 2.2.44 (0.3, 0.625, 1.25, 2.5 and 5 μM) and decitabine (Dec, nM). Y axis (Combination index (CI), CI<0.8=synergy), X axis (Fraction affected (Fa)).

FIG. 2. Graph showing combination effects of compound 2.2.44 (0.3, 0.625, 1.25, 2.5 and 5 μM) and 5-Azacytidine (5A). Y axis (Combination index (CI), CI<0.8=synergy), X axis (Fraction affected (Fa)).

FIG. 3. Graph showing combination effects of compound 2.2.45 (0.3, 0.625, 1.25, 2.5 and 5 μM) and decitabine (Dec, nM). Y axis (Combination index (CI), CI<0.8=synergy), X axis (Fraction affected (Fa)).

FIG. 4. Graph showing combination effects of compound 2.2.48 (0.3, 0.625, 1.25, 2.5 and 5 μM) and 5-Azacytidine (5A). Y axis (Combination index (CI), CI<0.8=synergy), X axis (Fraction affected (Fa)).

FIG. 5. Graph showing combination effects of compound 2.2.49 (0.3, 0.625, 1.25, 2.5 and 5 μM) and decitabine (Dec, nM). Y axis (Combination index (CI), CI<0.8=synergy), X axis (Fraction affected (Fa)).

FIG. 6. Graph showing combination effects of compound 2.2.49 (0.3, 0.625, 1.25, 2.5 and 5 μM) and 5-Azacytidine (5A). Y axis (Combination index (CI), CI<0.8=synergy), X axis (Fraction affected (Fa)).

FIG. 7. Graph showing combination effects of compound 2.2.50 (0.3, 0.625, 1.25, 2.5 and 5 μM) and decitabine (Dec, nM). Y axis (Combination index (CI), CI<0.8=synergy), X axis (Fraction affected (Fa)).

### DETAILED DESCRIPTION OF THE INVENTION

In a first aspect of the invention, there is provided a compound of formula I,

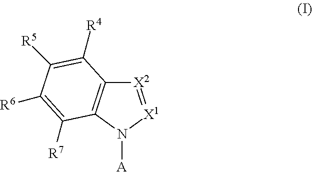

or a pharmaceutically acceptable salt thereof,
for use in the treatment of proliferative disorders, such as cancer and inflammation, wherein:
A represents -L¹-L²-L³-A¹;
A¹ represents aryl optionally substituted by one or more groups independently selected from Y¹ or heteroaryl optionally substituted by one or more groups independently selected from Y²;
each one of L¹ and L³ independently represents a single bond or C₁₋₃alkylene optionally substituted by one or more halo;
L² represents a single bond, —C(Q)-, —N(R¹)—, —O— or —S(O)ₙ—;
X¹ represents C(R²) or N;
X² represents C(R³) or N;
each R¹ and R³ independently represents H or C₁₋₆alkyl optionally substituted by one or more halo;
R² represents H, R^{a} or —OR^{b};
R⁴ and R⁷ independently represent H, halo, —CN, R^{c}, —N₃, —NO₂, —N(R^{d})R^{e}, —N(R^{f})C(Q′)R^{g}, —N(R^{h})S(O)ₙRⁱ, —OR^{j} or —SR^{k};
R⁵ and R⁶ independently represent H, halo, —CN, R^{c}, —N₃ or —NO₂; or
R⁴ and R⁵, R⁵ and R⁶ and/or R⁶ and R⁷ are linked together to form, along with the carbon atoms to which they are attached, a 5- or 6-membered ring, which ring optionally contains one to three heteroatoms and/or one or two further double bonds, and which ring optionally is substituted by one or more groups independently selected from halo, —OR^{j}, C₁₋₃alkyl optionally substituted by one or more halo, and Q¹;
Q represents ═O or ═S;
Q¹ represents ═O, ═NR^{r} or ═S;
R^{a} represents C₁₋₆alkyl optionally substituted by one or more groups independently selected from D¹, aryl optionally substituted by one or more groups independently selected from D² or heteroaryl optionally substituted by one or more groups independently selected from D³;
each R^{c} independently represents C₁₋₆alkyl optionally substituted by one or more halo;
each R^{b}, R^{d}, R^{e}, R^{f}, R^{g}, R^{h} and Rⁱ independently represents H or C₁₋₆alkyl optionally substituted by one or more halo; or
R^{d} and R^{e} are linked together to form, along with the nitrogen atom to which they are attached, a 3- to 6-membered ring, which ring optionally contains one further heteroatom and which ring optionally is substituted by one or more halo, one or more C₁₋₃alkyl each optionally and independently substituted by one or more F, or ═O;
D¹ represents halo, —OC₁₋₆alkyl optionally substituted by one or more halo, aryl optionally substituted by one or more groups independently selected from D² or heteroaryl optionally substituted by one or more groups independently selected from D³;
each D² and D³ independently represents halo, C₁₋₆alkyl optionally substituted by one or more halo or —OC₁₋₆alkyl optionally substituted by one or more halo;
each Y¹ and Y² independently represents halo, —BF₃M, —B(OR^{a1})₂, R^{b1}, —CN, —C(Q²)R^{c1}, —C(Q²)OR^{d1}, —C(Q²)N(R^{e1})R^{f1}, —N₃, —NO₂, —N(R^{g1})R^{h1}, —N(Rⁱ¹)C(Q²)R^{j1}, —N(R^{k1})C(Q²)N(R^{l1})R^{m1}, —N(Rⁿ¹)C(Q²)OR^{o1}, —N(R^{p1})S(O)ₙR^{q1}, —N(R^{r1})S(O)ₙN(R^{s1})R^{t1}, —OR^{u1}, —OC(Q²)R^{v1}, —OC(Q²)N(R^{w1})R^{x1}, —OC(Q²)OR^{y1}, —OS(O)ₙR^{z1}, —SR^{aa1}, —S(O)ₙR^{ab1}, —S(O)ₙN(R^{ac1})R^{ad1}, heterocycloalkyl optionally substituted by one or more groups independently selected from Z¹, aryl substituted by one or more groups independently selected from Z², heteroaryl optionally substituted by one or more groups independently selected from Z³ or Q²;
each Z¹ independently represents halo, R^{b1}, —CN, —C(Q²)R^{c1}, —C(Q²)OR^{d1}, —C(Q²)N(R^{e1})R^{f1}, —N₃, —NO₂, —N(R^{g1})R^{h1}, —N(Rⁱ¹)C(Q²)R^{j1}, —N(R^{k1})C(Q²)N(R^{l1})R^{m1}, —N(Rⁿ¹)C(Q²)OR^{o1}, —N(R^{p1})S(O)ₙR^{q1}, —N(R^{r1})S(O)ₙN(R^{s1})R^{t1}, —OR^{u1}, —OC(Q²)R^{v1}, —OC(Q²)N(R^{w1})R^{x1}, —OC(Q²)OR^{y1}, —OS(O)ₙR^{z1}, —SR^{aa1}, —S(O)ₙR^{ab1}, —S(O)ₙN(R^{ac1})R^{ad1} Or Q²;
each Z² and Z³ independently represents halo, —BF₃M, —B(OR^{a1})₂, R^{b1}, —CN, C(Q²)R^{c1}, —C(Q²)OR^{d1}, —C(Q²)N(R^{e1})R^{f1}, —N₃, —NO₂, —N(R^{g1})R^{h1}, —N(Rⁱ¹)C(Q²)R^{j1}, —N(R^{k1})C(Q²)N(R^{l1})R^{m1}, —N(Rⁿ¹)C(Q²)OR^{o1}, —N(R^{p1})S(O)ₙR^{q1}, —N(R^{r1})S(O)ₙN(R^{s1})R^{t1}, —OR^{u1}, —OC(Q²)R^{v1}, —OC(Q²)N(R^{w1})R^{x1}, —OC(Q²)OR^{y1}, —OS(O)ₙR^{z1}, —SR^{aa1}, —S(O)ₙR^{ab1} or —S(O)ₙN(R^{ac1})R^{ad1};
M represents a cation selected from (R^{M})₄N⁺, Li⁺, Na⁺, K⁺, Rb⁺ or Cs⁺;
each R^{M} independently represents C₁₋₁₂alkyl optionally substituted by one or more D⁴;
each Q² independently represents ═NR^{ae1}, ═N(OR^{af1}), ═O or ═S;
each R^{b1}, R^{o1}, R^{q1}, R^{y1}, R^{z1} and R^{ab1} independently represents C₁₋₄ alkyl optionally substituted by one or more groups independently selected from D⁴;
each R^{a1}, R^{c1}, R^{d1}, R^{e1}, R^{f1}, R^{g1}, R^{h1}, Rⁱ¹, R^{j1}, R^{k1}, R^{l1}, R^{m1}, Rⁿ¹, R^{p1}, R^{r1}, R^{s1}, R^{t1}, R^{u1}, R^{v1}, R^{w1}, R^{x1}, R^{aa1}, R^{ac1}, R^{ad1}, R^{ae1} and R^{af1} independently represents H or C₁₋₄alkyl optionally substituted by one or more groups independently selected from D⁴; or
R^{e1} and R^{f1}, R^{g1} and R^{h1}, R^{l1} and R^{m1}, R^{s1} and R^{t1}, R^{w1} and R^{x1} and/or R^{ac1} and R^{ad1} are linked together to form, along with the nitrogen atom to which they are attached, a 3- to 6-membered ring, which ring optionally contains one further heteroatom and which ring optionally is substituted by one or more groups independently selected from F, one or more C₁₋₃alkyl each optionally and independently substituted by one or more F, or ═O; or
two R^{a1} are linked together to form, along with the boron, and the oxygen atoms to which they are attached, a 5- to 8-membered heterocyclic ring, which ring optionally contains one or more further heteroatoms and which ring optionally is substituted by one or more groups independently selected from halo, one or more C₁₋₃alkyl optionally substituted by one or more halo, and/or one or more ═O;
each D⁴ independently represents halo, —OH or —OC₁₋₆alkyl optionally substituted by one or more halo;
each n independently represents 1 or 2; and
provided that at least one of R⁴ or R⁷ represents —NO₂.

These compounds are referred herein as compounds of the invention, or compounds for use according to the invention or compounds of the invention according to the first aspect of the invention.

In one embodiment the proliferative disorder is selected from cancer, and/or inflammation.

One embodiment refers to compounds of formula I according to the first aspect of the invention, wherein X¹ represents C(R²);

X² represents N;
R² represents H, R^{a} or —OR^{b};
R^{a} represents C₁₋₆alkyl (e.g. C₁₋₃alkyl) optionally substituted by one or more groups independently selected from D¹, or phenyl optionally substituted by one or two groups independently selected from D²;
R^{b} represents H or C₁₋₆alkyl (e.g. C₁₋₃alkyl) optionally substituted by one or more F;
D¹ represents F, —OC₁₋₄alkyl optionally substituted by one or more F, or phenyl optionally substituted by one or two groups independently selected from D²; and
D² represents F, Cl, C₁₋₄alkyl optionally substituted by one or more F or —OC₁₋₃alkyl optionally substituted by one or more F.

Another embodiment refers to compounds of formula I according to the first aspect of the invention, wherein X¹ represents C(R²);

X² represents N;
R² represents H, R^{a} or —OR^{b} (e.g. R^{a} or —OR^{b};
R^{a} represents:
(i) C₁₋₃alkyl optionally substituted by one to three F (e.g. methyl, ethyl, propyl, isopropyl, cyclopropyl, cyclopropylmethyl, difluoromethyl, trifluoromethyl or 2,2,2-trifluoroethyl) or —OC₁₋₃alkyl optionally substituted by one to three F (e.g. methoxymethyl, trifluoromethoxymethyl or ethoxyethyl);
(ii) —C₁₋₃alkylphenyl (e.g. benzyl) optionally substituted (e.g. not substituted) by one or two groups (e.g. one group) independently selected from F, Cl, methyl, difluoromethyl, trifluoromethyl, methoxy, difluoromethoxy and trifluoromethoxy; or
(iii) phenyl optionally substituted (e.g. not substituted) by one or two groups (e.g. one group) independently selected from F, Cl, methyl, difluoromethyl, trifluoromethyl, methoxy, difluoromethoxy and trifluoromethoxy; and
R^{b} represents C₁₋₂alkyl optionally substituted by one or more F (e.g. methyl, ethyl, difluoromethyl or trifluoromethyl).

A further embodiment refers to compounds of formula I according to the first aspect of the invention, wherein X¹ represents C(R²);

X² represents N; and
R² represents methyl, isopropyl, cyclopropyl, trifluoromethyl, methoxymethyl, benzyl or phenyl (e.g. methyl, cyclopropyl or trifluoromethyl).

One embodiment refers to compounds of formula I according to the first aspect of the invention, wherein X¹ represents C(R²);

X² represents C(R³);
R² represents H or R^{a};
R³ represents H or C₁₋₃alkyl optionally substituted by one or more F; and
R^{a} represents C₁₋₃alkyl optionally substituted by one or more F.

Another embodiment refers to compounds of formula I according to the first aspect of the invention, wherein X¹ represents C(R²);

X² represents C(H); and
R² represents H or C₁₋₃alkyl optionally substituted (e.g. unsubstituted, such as methyl) by one or more F (e.g. difluoromethyl or trifluoromethyl).

A further embodiment refers to compounds of formula I according to the first aspect of the invention, wherein X¹ represents N;

X² represents C(R³); and
R³ represents H or C₁₋₃alkyl optionally substituted by one or more F.

One embodiment refers to compounds of formula I according to the first aspect of the invention, wherein X¹ represents N;

X² represents C(R³); and
R³ represents C₁₋₃alkyl optionally substituted by one or more F (e.g. methyl, difluoromethyl or trifluoromethyl).

Another embodiment refers to compounds of formula I according to the first aspect of the invention, wherein X¹ represents N;

X² represents C(R³); and
R³ represents H.

A further embodiment refers to compounds of formula I according to the first aspect of the invention, wherein X¹ and X² represent N.

In one embodiment, X¹ represents C(R²).

In another embodiment, X¹ represents N.

In a further embodiment, X² represents C(R³).

In yet another embodiment, X² represents N.

In yet a further embodiment R⁴ represents —NO₂.

One embodiment refers to compounds of formula I according to the first aspect of the invention, wherein R⁴ represents —NO₂;

R⁵ and R⁶ independently represent H, halo, —CN, R^{c}, —N₃ or —NO₂;
R⁷ represents H; and
each R^{c} and R^{q} independently represents C₁₋₆alkyl optionally substituted by one or more F.

Another embodiment refers to compounds of formula I according to the first aspect of the invention, wherein R⁴ represents —NO₂;

R⁵ and R⁶ independently represent H, halo or R^{c};
R⁷ represents H; and
each R^{c} independently represents C₁₋₄alkyl optionally substituted by one or more F.

A further embodiment refers to compounds of formula I according to the first aspect of the invention, wherein R⁴ represents —NO₂;

R⁵ and R⁶ independently represent H, halo, methyl, difluoromethyl or trifluoromethyl; and
R⁷ represents H.

One embodiment refers to compounds of formula I according to the first aspect of the invention, wherein R⁴ represents —NO₂;

R⁵ and R⁶ represent H, F, Cl, or methyl, and where at least one of (e.g. both of)
R⁵ and R⁶ represent F, Cl, or methyl; and
R⁷ represents H.

Another embodiment refers to compounds of formula I according to the first aspect of the invention, wherein R⁴ represents —NO₂;

R⁵ and R⁶ or R⁶ and R⁷ (e.g. R⁵ and R⁶) are linked together to form, along with the carbon atoms to which they are attached, a 5- or 6-membered ring, which ring optionally contains one to three heteroatoms and/or one or two further double bonds, and which ring optionally is substituted by one or more groups independently selected from F and C₁₋₃alkyl optionally substituted by one or more F, and where the R⁵, R⁶ or R⁷ that is not part of the formed ring is represented by H, halo, —CN, R^{c}, —N₃ or —NO₂;
and
each R^{c} independently represents C₁₋₄alkyl optionally substituted by one or more F.

A further embodiment refers to compounds of formula I according to the first aspect of the invention, wherein R⁴ represents —NO₂;

R⁵ and R⁶ or R⁶ and R⁷ (e.g. R⁵ and R⁶), are linked together to form, along with the carbon atoms to which they are attached, a 5- or 6-membered ring, which ring optionally contains one heteroatom (e.g. no heteroatom) and/or one further double bond (eg. no further double bond), and which ring optionally is substituted by one or more groups independently selected from F and C₁₋₃alkyl optionally substituted by one or more F (e.g. F and/or methyl), and where the R⁵, R⁶ or R⁷ that is not part of the formed ring is represented by H, halo, —CN or R^{c}; and
each R^{c} represents C₁₋₄alkyl optionally substituted by one or more F (e.g. methyl).

One embodiment refers to compounds of formula I according to the first aspect of the invention, wherein R⁴ represents —NO₂;

R⁵ and R⁶ are linked together to form, along with the carbon atoms to which they are attached, a 5 or 6-membered ring (e.g. a 5-membered ring), which ring is optionally substituted by one or more (e.g. one, two, three or four) F or C₁₋₃alkyl optionally substituted by one or more F (e.g. methyl), (e.g. a 5-membered ring substituted by four methyl groups); and
R⁷ represents H.

In one embodiment of a compound of formula I according to the first aspect of the invention R⁷ represents —NO₂.

Another embodiment refers to compounds of formula I according to the first aspect of the invention, wherein R⁴ represents H;

R⁵ and R⁶ independently represent H, halo, —CN, R^{c}, —N₃ or —NO₂;
R⁷ represents —NO₂; and
each R^{c} and R^{q} independently represents C₁₋₆alkyl optionally substituted by one or more F.

A further embodiment refers to compounds of formula I, according to the first aspect of the invention, wherein R⁴ represents H;

R⁵ and R⁶ independently represent H, halo or R^{c};
R⁷ represents —NO₂; and
each R^{c} independently represents C₁₋₄alkyl optionally substituted by one or more F.

One embodiment refers to compounds of formula I according to the first aspect of the invention, wherein R⁴ represents H;

R⁵ and R⁶ independently represent H, halo, methyl, difluoromethyl or trifluoromethyl; and
R⁷ represents —NO₂.

Another embodiment refers to compounds of formula I according to the first aspect of the invention, wherein R⁴ represents H;

R⁵ and R⁶ represent H, F, Cl, or methyl, and where at least one of (e.g. both of) R⁵ and R⁶ represent F, Cl, or methyl; and
R⁷ represents —NO₂.

Another embodiment refers to compounds of formula I, according to the first aspect of the invention, wherein

R⁴ and R⁵ or R⁵ and R⁶ (e.g. R⁵ and R⁶) are linked together to form, along with the carbon atoms to which they are attached, a 5- or 6-membered ring, which ring optionally contains one to three heteroatoms and/or one or two further double bonds, and which ring optionally is substituted by one or more groups independently selected from F and C₁₋₃alkyl optionally substituted by one or more F, and where the R⁴, R⁵ or R⁶ that is not part of the formed ring is represented by H, halo, —CN, R^{c}, —N₃ or —NO₂;
R⁷ represents —NO₂; and
each R^{c} independently represents C₁₋₄alkyl optionally substituted by one or more F.

A further embodiment refers to compounds of formula I according to the first aspect of the invention, wherein

R⁴ and R⁵ or R⁵ and R⁶ (e.g. R⁵ and R⁶) are linked together to form, along with the carbon atoms to which they are attached, a 5- or 6-membered ring, which ring optionally contains one heteroatom (e.g. no heteroatom) and/or one further double bond (eg. no further double bond), and which ring optionally is substituted by one or more groups independently selected from F and C₁₋₃alkyl optionally substituted by one or more F (e.g. F and/or methyl), and where the R⁴, R⁵ or R⁷ that is not part of the formed ring is represented by H, halo, —CN or R^{c};
R⁷ represents —NO₂; and
each R^{c} represents C₁₋₄alkyl optionally substituted by one or more F (e.g. methyl).

Another embodiment refers to compounds of formula I according to the first aspect of the invention, wherein R⁴ represents H;

R⁵ and R⁶ are linked together to form, along with the carbon atoms to which they are attached, a 5 or 6-membered ring (e.g. a 5-membered ring), which ring is optionally substituted by one or more (e.g. one, two, three or, four) F, or C₁₋₃alkyl optionally substituted by one or more F (e.g. methyl), (e.g. a 5-membered ring substituted by four methyl groups); and
R⁷ represents —NO₂.

In one embodiment, when R⁵ and R⁶ are not linked together to form, along with the carbon atoms to which they are attached, a 5- or 6-membered ring, then R⁵ and R⁶ are independently selected from H, halo and R^{c}. For example, R⁵ and R⁶ may be independently selected from H, F, Cl, methyl and trifluoromethyl. In one embodiment R⁵ and R⁶ are independently selected from F, Cl, methyl and trifluoromethyl. In another embodiment of the first aspect of the invention R⁵ and R⁶ are the same, e.g. F, Cl or methyl.

Another embodiment refers to compounds of formula I according to the first aspect of the invention, wherein A represents -L¹-L²-L³-A¹;

each one of L¹ and L³ independently represents a single bond or C₁₋₃alkylene; and
L² represents a single bond, —C(O)— or —S(O)₂—.

A further embodiment refers to compounds of formula I according to the first aspect of the invention, wherein A represents -L¹-L²-L³-A¹;

each one of L¹ and L³ independently represents a single bond, —CH₂—, —CH₂CH₂— or —CH(Me)-; and
L² represents a single bond, —C(O)— or —S(O)₂—.

One embodiment refers to compounds of formula I according to the first aspect of the invention, wherein A represents -L¹-L²-L³-A¹; and

-L¹-L²-L³- represents a single bond, —CH₂—, —CH₂CH₂—, —CH(Me)-, —C(O)— or —S(O)₂—.

Another embodiment refers to compounds of formula I according to the first aspect of the invention, wherein A represents —CH₂-A¹.

A further embodiment refers to compounds of formula I according to the first aspect of the invention, wherein A¹ represents phenyl optionally substituted by one to three groups independently selected from Y¹, or heteroaryl optionally substituted by one to three groups independently selected from Y²;

each Y¹ and Y² independently represents halo, R^{b1}, —CN, —C(Q²)R^{c1}, —C(O)OR^{d1}, —C(O)N(R^{e1})R^{f1}, —N(Rⁱ¹)C(o)R^{j1}, —N(R^{p1})S(O)₂R^{q1}, —OR^{u1}, —OC(O)R^{v1}, —S(O)₂R^{ab1} or heteroaryl optionally substituted by one or more groups independently selected from Z³;
each Z³ independently represents halo (e.g. F or Cl, for example Cl) or C₁₋₃alkyl optionally substituted (e.g. unsubstituted, e.g. methyl) by one or more F (e.g. difluoromethyl or trifluoromethyl);
Q² represents ═O or ═N(OH);
each R^{b1} independently represents F, —OH or -OMe;
each R^{q1} and R^{ab1} independently represents C₁₋₃alkyl optionally substituted by one or more F; and
each R^{c1}, R^{d1}, R^{e1}, R^{f1}, Rⁱ¹, R^{j1}, R^{p1}, R^{r1}, R^{u1} and R^{v1} independently represents H or C₁₋₃alkyl optionally substituted by one or more F.

One embodiment refers to compounds of formula I according to the first aspect of the invention, wherein A¹ represents:
(i) phenyl optionally substituted (e.g. in the meta- and/or para-position, for example in the para position, or not substituted) by one, two or three (e.g. one) F, Cl, Br, —CN, —CH(OH)CH═CH₂, —C(═NOH)H, —C(O)H, —C(O)NH₂, —C(O)OH, —C(O)OMe-NH₂, —N(H)C(O)Me, —N(H)C(O)CH═CH₂, —OH, -OMe, —OCF₃, —OC(O)Me, —S(O)₂Me, pyridinyl (e.g. 2-chloro-4-pyridinyl), pyrrolyl (e.g. pyrrol-1-yl), thiazolyl (e.g. 2-methylthiazol-4-yl) or 1,2,4-triazol-1-yl; or
(ii) heteroaryl (e.g. pyrazolyl or benzodioxolyl optionally substituted by halo or C₁₋₃alkyl optionally substituted by one or more F (e.g. 3,5-dimethylpyrazol-4-yl or 6-chlorobenzodioxol-5-yl).

Another embodiment refers to compounds of formula I, according to the first aspect of the invention, wherein A¹ represents:
(i) phenyl substituted by —BF₃K or —B(OR^{a1})₂ (e.g. —B(OR^{a1})₂) and optionally substituted by one or more groups independently selected from F, Cl, methyl, difluoromethyl, trifluoromethyl, —OH and -OMe;
(ii) monocyclic heteroaryl substituted by —BF₃K or —B(OR^{a1})₂ (e.g. —B(OR^{a1})₂) and optionally substituted by one or more groups independently selected from F, Cl, methyl, difluoromethyl, trifluoromethyl, —OH and -OMe; or
(iii) bicyclic boron containing partly aromatic heteroaryl, (e.g. 1,3-dihydrobenzo-[c][1,2]oxaborolyl or 1,2-dihydrobenzo[d][1,2,3]diazaborininyl substituted on the boron by —OH and optionally substituted by one or more groups independently selected from F, Cl, methyl, difluoromethyl, trifluoromethyl, —OH and -OMe;
   each R^{a1} independently represents H or C₁₋₃alkyl optionally substituted by one or more F; or
   two R^{a1} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O.

A further embodiment refers to compounds of formula I according to the first aspect of the invention, wherein A¹ represents phenyl substituted by —BF₃K or —B(OR^{a1})₂ (e.g. —B(OR^{a1})₂) and optionally substituted by one or more groups independently selected from F, Cl, methyl, —OH and -OMe;

each R^{a1} independently represents H or C₁₋₃alkyl optionally substituted by one or more F; or
two R^{a1} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O.

One embodiment refers to compounds of formula I according to the first aspect of the invention, wherein A¹ represents phenyl substituted in the meta-, or para-position (e.g. in the para-position) by —B(OH)₂, —B(OMe)₂, 4,4,5,5-tetramethyl-1,3,2-dioxaborolan-2-yl or 6-methyl-1,3,6,2-dioxazaborocane-4,8-dion-2-yl.

Another embodiment refers to compounds of formula I, wherein A¹ represents phenyl substituted by 6-methyl-1,3,6,2-dioxazaborocane-4,8-dion-2-yl.

A further embodiment refers to compounds of formula I according to the first aspect of the invention, wherein A represents -L¹-L²-L³-A¹;

each one of L¹ and L³ independently represents a single bond or C₁₋₃alkylene;
L² represents a single bond, —C(O)— or —S(O)₂—;
A¹ represents phenyl optionally substituted by one to three groups independently selected from Y¹, or heteroaryl optionally substituted by one to three groups independently selected from Y²;
X¹ represents C(R²);
X² represents N;
R² represents H, R^{a} or —OR^{b};
R⁴ represents —NO₂;
R⁵ and R⁶ independently represent H, halo or R^{c};
R⁷ represents H; or
R⁵ and R⁶, or R⁶ and R⁷ (e.g. R⁵ and R⁶) are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F or R^{c};
R^{a} represents C₁₋₆alkyl (e.g. C₁₋₄alkyl) optionally substituted by one or more groups independently selected from D¹, or phenyl optionally substituted by one or two groups independently selected from D²;
R^{b} represents H or C₁₋₆alkyl (e.g. C₁₋₄alkyl) optionally substituted by one or more F;
each R^{c} independently represents C₁₋₆alkyl optionally substituted by one or more F;
D¹ represents F, —OC₁₋₄alkyl optionally substituted by one or more F, or phenyl optionally substituted by one or two groups independently selected from D²;
D² represents F, Cl, C₁₋₄alkyl optionally substituted by one or more F or —OC₁₋₃alkyl optionally substituted by one or more F;
each Y¹ and Y² independently represents halo, R^{b1}, —CN, —C(Q²)R^{c1}, —C(O)OR^{d1}, —C(O)N(R^{e1})R^{f1}, —N(Rⁱ¹)C(O)R^{j1}, —N(R^{p1})S(O)₂R^{q1}, —OR^{u1}, —OC(O)R^{v1}, —S(O)₂R^{ab1} or heteroaryl optionally substituted by one or more groups independently selected from Z³;
each Z³ independently represents halo (e.g. F or Cl) or C₁₋₃alkyl optionally substituted (e.g. unsubstituted, e.g. methyl) by one or more F (e.g. difluoromethyl or trifluoromethyl);
Q² represents ═O or ═N(OH);
each R^{b1}, R^{q1} and R^{ab1} independently represents C₁₋₃alkyl optionally substituted by one or more F; and
each R^{c1}, R^{d1}, R^{e1}, R^{f1}, Rⁱ¹, R^{j1}, R^{p1}, R^{r1}, R^{u1} or R^{v1} independently represents H or C₁₋₃alkyl optionally substituted by one or more F.

One embodiment refers to compounds of formula I according to the first aspect of the invention, wherein A represents -L¹-L²-L³-A¹;

-L¹-L²-L³- represents a single bond, —CH₂—, —CH₂CH₂—, —CH(Me)-, —C(O)— or —S(O)₂—;
A¹ represents:
(i) phenyl optionally substituted in the meta- and/or para-position, (e.g. in the para position, or not substituted) by one, two or three (e.g. one) F, Cl, Br, —CN, —CH(OH)CH═CH₂, —C(═NOH)H, —C(O)H, —C(O)NH₂, —C(O)OH, —C(O)OMe, —NH₂, —N(H)C(O)Me, —N(H)C(O)CH═CH₂, —OH, -OMe, —OCF₃, —OC(O)Me, —S(O)₂Me, pyridinyl (e.g. 2-chloro-4-pyridinyl), pyrrolyl (e.g. pyrrol-1-yl), thiazolyl (e.g. 2-methylthiazol-4-yl) or 1,2,4-triazol-1-yl; or
(ii) heteroaryl (e.g. pyrazolyl or benzodioxolyl) optionally substituted by halo or C₁₋₃alkyl optionally substituted by one or more F (e.g. 3,5-dimethylpyrazol-4-yl or 6-chlorobenzodioxol-5-yl);
   X¹ represents C(R²);
   X² represents N;
   R² represents R^{a} or —OR^{b};
   R⁴ represents —NO₂;
   R⁵ and R⁶ independently represents H, halo or R^{c};
   R⁷ represents H; or
   R⁵ and R⁶, or R⁶ and R⁷ (e.g. R⁵ and R⁶) are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F or R^{c};
   R^{a} represents:
(i) C₁₋₄alkyl optionally substituted by one to three F (e.g. methyl, ethyl, propyl, isopropyl, cyclopropyl, cyclopropylmethyl, difluoromethyl, trifluoromethyl or 2,2,2-trifluoroethyl) or —OC₁₋₃alkyl optionally substituted by one to three F (e.g. methoxymethyl, trifluoromethoxymethyl or ethoxyethyl);
(ii) C₁₋₃alkylphenyl (e.g. benzyl) optionally substituted (e.g. not substituted) by one or two (e.g. one) groups independently selected from F, Cl, methyl, difluoromethyl, trifluoromethyl, methoxy, difluoromethoxy and trifluoromethoxy); or
(iii) phenyl optionally substituted (e.g. not substituted) by one or two (e.g. one) groups independently selected from F, Cl, methyl, difluoromethyl, trifluoromethyl, methoxy, difluoromethoxy and trifluoromethoxy;
   R^{b} represents C₁₋₂alkyl optionally substituted by one or more F (e.g. methyl, ethyl, difluoromethyl or trifluoromethyl); and
   each R^{c} independently represents C₁₋₆alkyl optionally substituted by one or more F.

Another embodiment refers to compounds of formula I according to the first aspect of the invention, wherein A represents -L¹-L²-L³-A¹;

each one of L¹ and L³ independently represents a single bond or C₁₋₃alkylene;
L² represents a single bond, —C(O)— or —S(O)₂—;
A¹ represents:
(i) phenyl substituted by —BF₃K or —B(OR^{a1})₂ (e.g. —B(OR^{a1})₂) and optionally substituted by F, Cl, methyl, difluoromethyl, trifluoromethyl, —OH or -OMe;
(ii) monocyclic heteroaryl substituted by —BF₃K or —B(OR^{a1})₂ (e.g. —B(OR^{a1})₂) and optionally substituted by F, Cl, methyl, difluoromethyl, trifluoromethyl, —OH or -OMe; or
(iii) bicyclic, boron containing, partly aromatic heteroaryl, (e.g. 1,3-dihydrobenzo[c][1,2]oxaborolyl or 1,2-dihydrobenzo[d][1,2,3]diazaborininyl) substituted on the boron by —OH and optionally substituted by one or more groups independently selected from F, Cl, methyl, difluoromethyl, trifluoromethyl, —OH and -OMe;
   X¹ represents C(R²);
   X² represents N;
   R² represents R^{a} or —OR^{b};
   R⁴ represents —NO₂;
   R⁵ and R⁶ independently represent H, halo or R^{c};
   R⁷ represents H; or
   R⁵ and R⁶, or R⁶ and R⁷ (e.g. R⁵ and R⁶) are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F and R^{c};
   R^{a} represents:
(i) C₁₋₄alkyl optionally substituted by one to three F (e.g. methyl, ethyl, propyl, isopropyl, cyclopropyl, cyclopropylmethyl, difluoromethyl, trifluoromethyl or 2,2,2-trifluoroethyl) or —OC₁₋₃alkyl optionally substituted by one to three F (e.g. methoxymethyl, trifluoromethoxymethyl or ethoxyethyl);
(ii) C₁₋₃alkylphenyl (e.g. benzyl) optionally substituted (e.g. not substituted) by one or two (e.g. one) groups independently selected from F, Cl, methyl, difluoromethyl, trifluoromethyl, methoxy, difluoromethoxy and trifluoromethoxy; or
(iii) phenyl optionally substituted (e.g. not substituted) by one or two (e.g. one) groups independently selected from F, Cl, methyl, difluoromethyl, trifluoromethyl, methoxy, difluoromethoxy and trifluoromethoxy;
   R^{b} represents C₁₋₂alkyl optionally substituted by one or more F (e.g. methyl, ethyl, difluoromethyl or trifluoromethyl);
   each R^{c} independently represents C₁₋₆alkyl optionally substituted by one or more F;
   each R^{a1} independently represents H or C₁₋₃alkyl optionally substituted by one or more F; or
   two R^{a1} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O.

A further embodiment refers to compounds of formula I according to the first aspect of the invention, wherein A represents -L¹-L²-L³-A¹;

-L¹-L²-L³- represents:
(i) a single bond;
(ii) —CH₂—;
(iii) —CH₂CH₂— or —CH(Me)-; or
(iv) —C(O)— or —S(O)₂—;
   A¹ represents phenyl substituted by —BF₃K or —B(OR^{a1})₂ (e.g. —B(OR^{a1})₂) and optionally substituted by F, Cl, methyl, —OH or -OMe;
   X¹ represents C(R²);
   X² represents N;
   R² represents R^{a} or —OR^{b};
   R⁴ represents —NO₂;
   R⁵ and R⁶ independently represents H, halo or R^{c};
   R⁷ represents H; or
   R⁵ and R⁶, or R⁶ and R⁷ (e.g. R⁵ and R⁶) are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F and R^{c};
   R^{a} represents:
(i) C₁₋₄alkyl optionally substituted by one to three F (e.g. methyl, ethyl, propyl, isopropyl, cyclopropyl, cyclopropylmethyl, difluoromethyl, trifluoromethyl or 2,2,2-trifluoroethyl) or —OC₁₋₃alkyl optionally substituted by one to three F (e.g. methoxymethyl, trifluoromethoxymethyl or ethoxyethyl);
(ii) C₁₋₃alkylphenyl (e.g. benzyl) optionally substituted (e.g. not substituted) by one or two (e.g. one) groups independently selected from F, Cl, methyl, difluoromethyl, trifluoromethyl, methoxy, difluoromethoxy and trifluoromethoxy; or
(iii) phenyl optionally substituted (e.g. not substituted) by one or two (e.g. one) groups independently selected from F, Cl, methyl, difluoromethyl, trifluoromethyl, methoxy, difluoromethoxy and trifluoromethoxy;
   R^{b} represents C₁₋₂alkyl optionally substituted by one or more F (e.g. methyl, ethyl, difluoromethyl or trifluoromethyl);
   each R^{c} independently represents C₁₋₆alkyl optionally substituted by one or more F;
   each R^{a1} independently represents H or C₁₋₃alkyl optionally substituted by one or more F; or
   two R^{a1} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O.

In an embodiment of the invention -L¹-L²-L³- represents —CH₂CH₂— or —CH(Me)-. In another embodiment of the invention -L¹-L²-L³- represents —CH₂—.

One embodiment refers to compounds of formula I, according to the first aspect of the invention, wherein A represents —CH₂-A¹;

A¹ represents phenyl substituted in the meta- or para-position (e.g. in the para-position) by —B(OH)₂, —B(OMe)₂, 4,4,5,5-tetramethyl-1,3,2-dioxaborolan-2-yl or 6-methyl-1,3,6,2-dioxazaborocane-4,8-dion-2-yl;
X¹ represents C(R²);
X² represents N;
R² represents methyl, isopropyl, cyclopropyl, trifluoromethyl, methoxymethyl, benzyl or phenyl (e.g. methyl, cyclopropyl or trifluoromethyl);
R⁴ represents —NO₂;
R⁵ and R⁶ represent H, F, Cl, or methyl, and where at least one of (e.g. both of) R⁵ and R⁶ represent F, Cl, or methyl; and
R⁷ represents H.

Another embodiment refers to compounds of formula I according to the first aspect of the invention, wherein A represents -L¹-L²-L³-A¹;

each one of L¹ and L³ independently represents a single bond or C₁₋₃alkylene;
L² represents a single bond, —C(O)— or —S(O)₂—;
A¹ represents phenyl optionally substituted by one to three groups independently selected from Y¹, or heteroaryl optionally substituted by one to three groups independently selected from Y²;
X¹ represents C(R²);
X² represents N;
R² represents H, R^{a} or —OR^{b};
R⁴ represents H;
R⁵ and R⁶ independently represents H, halo or R^{c};
or
R⁴ and R⁵, or R⁵ and R⁶ (e.g. R⁵ and R⁶), are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F and R^{c};
R⁷ represents —NO₂;
R^{a} represents C₁₋₆alkyl (e.g. C₁₋₄alkyl) optionally substituted by one or more groups independently selected from D¹, or phenyl optionally substituted by one or two groups independently selected from D²;
R^{b} represents H or C₁₋₆alkyl (e.g. C₁₋₄alkyl) optionally substituted by one or more F;
each R^{c} independently represents C₁₋₆alkyl optionally substituted by one or more F;
D¹ represents F, —OC₁₋₄alkyl optionally substituted by one or more F, or phenyl optionally substituted by one or two groups independently selected from D²;
D² represents F, Cl, C₁₋₄alkyl optionally substituted by one or more F or —OC₁₋₃alkyl optionally substituted by one or more F;
each Y¹ and Y² independently represents halo, R^{b1}, —CN, —C(Q²)R^{c1}, —C(O)OR^{d1}, —C(O)N(R^{e1})R^{f1}, —N(Rⁱ¹)C(O)R^{j1}, —N(R^{p1})S(O)₂R^{q1}, —OR^{u1}, —OC(O)R^{v1}, —S(O)₂R^{ab1} or heteroaryl optionally substituted by one or more groups independently selected from Z³;
each Z³ independently represents halo (such as F or Cl, e.g. Cl) or C₁₋₃alkyl optionally substituted (e.g unsubstituted, i.e. methyl) by one or more F (e.g. difluoromethyl or trifluoromethyl);
Q² represents ═O or ═N(OH);
each R^{b1}, R^{q1} and R^{ab1} independently represents C₁₋₃alkyl optionally substituted by one or more F; and
each R^{c1}, R^{d1}, R^{e1}, R^{f1}, Rⁱ¹, R^{j1}, R^{p1}, R^{r1}, R^{u1} or R^{v1} independently represents H or C₁₋₃alkyl optionally substituted by one or more F.

A further embodiment refers to compounds of formula I according to the first aspect of the invention, wherein A represents -L¹-L²-L³-A¹;

-L¹-L²-L³- represents a single bond, —CH₂—, —CH₂CH₂—, —CH(Me)-, —C(O)— or —S(O)₂—;
A¹ represents:
(i) phenyl optionally substituted (e.g. not substituted) in the ortho-, meta- and/or para-position (e.g. in the meta or para position) by one, two or three (e.g. one) F, Cl, Br, —CN, —CH(OH)CH═CH₂, —C(═NOH)H, —C(O)H, —C(O)NH₂, —C(O)OH, —C(O)OMe, —NH₂, —N(H)C(O)Me, —N(H)C(O)CH═CH₂, —OH, -OMe, —OCF₃, —OC(O)Me, —S(O)₂Me, pyridinyl (e.g. 2-chloro-4-pyridinyl), pyrrolyl (e.g. pyrrol-1-yl), thiazolyl (e.g. 2-methylthiazol-4-yl) or 1,2,4-triazol-1-yl; or
(ii) heteroaryl (e.g. pyrazolyl or benzodioxolyl) optionally substituted by halo or C₁₋₃alkyl optionally substituted by one or more F (e.g. 3,5-dimethylpyrazol-4-yl or 6-chlorobenzodioxol-5-yl);
   X¹ represents C(R²);
   X² represents N;
   R² represents R^{a} or —OR^{b};
   R⁴ represents —H;
   R⁵ and R⁶ independently represent H, halo or R^{c}; or
   R⁴ and R⁵,or R⁵ and R⁶ (e.g. R⁵ and R⁶) are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F and R^{c};
   R⁷ represents —NO₂;
   R^{a} represents:
(i) C₁₋₄alkyl optionally substituted by one to three F (e.g. methyl, ethyl, propyl, isopropyl, cyclopropyl, cyclopropylmethyl, difluoromethyl, trifluoromethyl or 2,2,2-trifluoroethyl) or —OC₁₋₃alkyl optionally substituted by one to three F (e.g. methoxymethyl, trifluoromethoxymethyl or ethoxyethyl);
(ii) —C₁₋₃alkylphenyl (e.g. benzyl) optionally substituted (e.g. not substituted) by one or two (e.g. one) groups independently selected from F, Cl, methyl, difluoromethyl, trifluoromethyl, methoxy, difluoromethoxy and trifluoromethoxy; or
(iii) phenyl optionally substituted (e.g. not substituted) by one or two (e.g one) groups independently selected from F, Cl, methyl, difluoromethyl, trifluoromethyl, methoxy, difluoromethoxy and trifluoromethoxy;
   R^{b} represents C₁₋₂alkyl optionally substituted by one or more F (e.g. methyl, ethyl, difluoromethyl or trifluoromethyl); and
   each R^{c} independently represents C₁₋₆alkyl optionally substituted by one or more F.

One embodiment refers to compounds of formula I according to the first aspect of the invention, wherein A represents -L¹-L²-L³-A¹;

each one of L¹ and L³ independently represents a single bond or C₁₋₃alkylene;
L² represents a single bond, —C(O)— or —S(O)₂—;
A¹ represents:
(i) phenyl substituted by —BF₃K or —B(OR^{a1})₂ (e.g. —B(OR^{a1})₂) and optionally substituted by F, Cl, methyl, difluoromethyl, trifluoromethyl, —OH or -OMe;
(ii) monocyclic heteroaryl substituted by —BF₃K or —B(OR^{a1})₂ (e.g. —B(OR^{a1})₂) and optionally substituted by F, Cl, methyl, difluoromethyl, trifluoromethyl, —OH or -OMe; or
(iii) bicyclic, boron containing, partly aromatic heteroaryl, (e.g. 1,3-dihydrobenzo[c][1,2]oxaborolyl or 1,2-dihydrobenzo[d][1,2,3]diazaborininyl) substituted on the boron by —OH and optionally substituted by one or more groups independently selected from F, Cl, methyl, difluoromethyl, trifluoromethyl, —OH and -OMe;
   X¹ represents C(R²);
   X² represents N;
   R² represents R^{a} or —OR^{b};
   R⁴ represents H;
   R⁵ and R⁶ independently represents H, halo or R^{c}; or
   R⁴ and R⁵, or R⁵ and R⁶ (e.g. R⁵ and R⁶) are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F and R^{c};
   R⁷ represents —NO₂;
   R^{a} represents:
(i) C₁₋₄alkyl optionally substituted by one to three F (e.g. methyl, ethyl, propyl, isopropyl, cyclopropyl, cyclopropylmethyl, difluoromethyl, trifluoromethyl or 2,2,2-trifluoroethyl) or —OC₁₋₃alkyl optionally substituted by one to three F (e.g. methoxymethyl, trifluoromethoxymethyl or ethoxyethyl);
(ii) C₁₋₃alkylphenyl (e.g. benzyl) optionally substituted (e.g. not substituted) by one or two (e.g. one) groups independently selected from F, Cl, methyl, difluoromethyl, trifluoromethyl, methoxy, difluoromethoxy and trifluoromethoxy; or
(iii) phenyl optionally substituted (e.g. not substituted) by one or two (e.g. one) groups independently selected from F, Cl, methyl, difluoromethyl, trifluoromethyl, methoxy, difluoromethoxy and trifluoromethoxy;
   R^{b} represents C₁₋₂alkyl optionally substituted by one or more F (e.g. methyl, ethyl, difluoromethyl or trifluoromethyl);
   each R^{c} independently represents C₁₋₆alkyl optionally substituted by one or more F;
   each R^{a1} independently represents H or C₁₋₃alkyl optionally substituted by one or more F; or
   two R^{a1} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O.

Another embodiment refers to compounds of formula I according to the first aspect of the invention, wherein A represents -L¹-L²-L³-A¹;

-L¹-L²-L³- represents:
(i) a single bond;
(ii) —CH₂—;
(iii) —CH₂CH₂— or —CH(Me)-; or
(iv) —C(O)— or —S(O)₂—;
   A¹ represents phenyl substituted by —BF₃K or —B(OR^{a1})₂ (e.g. —B(OR^{a1})₂) and optionally substituted by F, Cl, methyl, —OH or -OMe;
   X¹ represents C(R²);
   X² represents N;
   R² represents R^{a} or —OR^{b};
   R⁴ represents H;
   R⁵ and R⁶ independently represents H, halo or R^{c}; or
   R⁴ and R⁵, or R⁵ and R⁶ (e.g., R⁵ and R⁶) are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F and R^{c};
   R⁷ represents —NO₂;
   R^{a} represents:
(i) C₁₋₄alkyl optionally substituted by one to three F (e.g. methyl, ethyl, propyl, isopropyl, cyclopropyl, cyclopropylmethyl, difluoromethyl, trifluoromethyl or 2,2,2-trifluoroethyl) or —OC₁₋₃alkyl optionally substituted by one to three F (e.g. methoxymethyl, trifluoromethoxymethyl or ethoxyethyl);
(ii) C₁₋₃alkylphenyl (e.g. benzyl) optionally substituted (e.g. not substituted) by one or two, (e.g. one) groups independently selected from F, Cl, methyl, difluoromethyl, trifluoromethyl, methoxy, difluoromethoxy and trifluoromethoxy; or
(iii) phenyl optionally substituted (e.g. not substituted) by one or two, (e.g. one) groups independently selected from F, Cl, methyl, difluoromethyl, trifluoromethyl, methoxy, difluoromethoxy and trifluoromethoxy);
   R^{b} represents C₁₋₂alkyl optionally substituted by one or more F (e.g. methyl, ethyl, difluoromethyl or trifluoromethyl);
   each R^{c} independently represents C₁₋₆alkyl optionally substituted by one or more F;
   each R^{a1} independently represents H or C₁₋₃alkyl optionally substituted by one or more F; or
   two R^{a1} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O.

A further embodiment refers to compounds of formula I according to the first aspect of the invention, wherein A represents —CH₂-A¹;

A¹ represents phenyl substituted in the meta- or para-position (e.g. in the para-position) by —B(OH)₂, —B(OMe)₂, 4,4,5,5-tetramethyl-1,3,2-dioxaborolan-2-yl or 6-methyl-1,3,6,2-dioxazaborocane-4,8-dion-2-yl;
X¹ represents C(R²);
X² represents N;
R² represents methyl, isopropyl, cyclopropyl, trifluoromethyl, methoxymethyl, benzyl or phenyl (e.g. methyl, cyclopropyl or trifluoromethyl);
R⁴ represents H;
R⁵ and R⁶ represent H, F, Cl or methyl, and where at least one of (e.g. both of) R⁵ and R⁶ represent F, Cl, or methyl;
R⁷ represents —NO₂.

One embodiment refers to compounds of formula I according to the first aspect of the invention, wherein A represents -L¹-L²-L³-A¹;

each one of L¹ and L³ independently represents a single bond or C₁₋₃alkylene;
L² represents a single bond, —C(O)— or —S(O)₂—;
A¹ represents phenyl optionally substituted by one to three groups independently selected from Y¹, or heteroaryl optionally substituted by one to three groups independently selected from Y²;
X¹ represents C(R²);
X² represents C(R³);
R² and R³ independently represents H or R^{a};
R⁴ represents —NO₂;
R⁵ and R⁶ independently represents H, halo or R^{c};
R⁷ represents H; or
R⁵ and R⁶, or R⁶ and R⁷ (e.g. R⁵ and R⁶) are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F and R^{c};
each R^{a} and R^{c} independently represents C₁₋₄alkyl optionally substituted by one or more F;
each R^{d}, R^{e}, R^{f}, R^{g}, R^{h}, Rⁱ, R^{j}, R^{k}, R^{l}, R^{m}, Rⁿ, R^{o} and R^{r} independently represents H or C₁₋₆alkyl optionally substituted by one or more F; or
R^{d} and R^{e} and/or R^{o} and R^{P} are linked together to form, along with the nitrogen atom to which they are attached, a 3- to 6-membered ring, which ring optionally contains one further heteroatom and which ring optionally is substituted by one or more F, one or more C₁₋₃alkyl each optionally and independently substituted by one or more F, or ═O;
each Y¹ and Y² independently represents halo, R^{b1}, —CN, —C(Q²)R^{c1}, —C(O)OR^{d1}, —C(O)N(R^{e1})R^{f1}, —N(Rⁱ¹)C(O)R^{j1}, —N(R^{p1})S(O)₂R^{q1}, —OR^{u1}, —OC(O)R^{v1}, —S(O)₂R^{ab1} or heteroaryl optionally substituted by one or more groups independently selected from Z³;
each Z³ independently represents halo (e.g. F or Cl) or C₁₋₃alkyl optionally substituted (e.g unsubstituted, e.g. methyl) by one or more F (e.g. difluoromethyl or trifluoromethyl);
Q² represents ═O or ═N(OH);
each R^{b1}, R^{q1} and R^{ab1} independently represents C₁₋₃alkyl optionally substituted by one or more F; and
each R^{c1}, R^{d1}, R^{e1}, R^{f1}, Rⁱ¹, R^{j1}, R^{p1}, R^{r1}, R^{u1} or R^{V1} independently represents H or C₁₋₃alkyl optionally substituted by one or more F.

Another embodiment refers to compounds of formula I according to the first aspect of the invention, wherein A represents -L¹-L²-L³-A¹;

-L¹-L²-L³- represents:
(i) a single bond;
(ii) —CH₂—;
(iii) —CH₂CH₂— or —CH(Me)-; or
(iv) —C(O)— or —S(O)₂—;
   A¹ represents phenyl optionally substituted (e.g. in the para position) by one or two (e.g. one) groups independently selected from F, Cl, C₁₋₃alkyl optionally substituted by one or more F and, —OC₁₋₃alkyl optionally substituted by one or more F (e.g. -OMe);
   X¹ represents C(R²);
   X² represents C(R³);
   R² and R³ independently represent H or C₁₋₃alkyl optionally substituted by one or more F (e.g. methyl or trifluoromethyl);
   R⁴ represents —NO₂;
   R⁵ and R⁶ independently represent H, halo or R^{c};
   R⁷ represents H; and
   each R^{c} independently represents C₁₋₃alkyl optionally substituted by one or more F.

A further embodiment refers to compounds of formula I according to the first aspect of the invention, wherein A represents -L¹-L²-L³-A¹;

each one of L¹ and L³ independently represents a single bond or C₁₋₃alkylene;
L² represents a single bond, —C(O)— or —S(O)₂—;
A¹ represents:
(i) phenyl substituted by —BF₃K or —B(OR^{a1})₂, (e.g. —B(OR^{a1})₂) and optionally substituted by F, Cl, methyl, difluoromethyl, trifluoromethyl, —OH or -OMe;
(ii) monocyclic heteroaryl substituted by —BF₃K or —B(OR^{a1})₂ (e.g. —B(OR^{a1})₂) and optionally substituted by F, Cl, methyl, difluoromethyl, trifluoromethyl, —OH or -OMe; or
(iii) bicyclic, boron containing, partly aromatic heteroaryl, (e.g. 1,3-dihydrobenzo[c][1,2]oxaborolyl or 1,2-dihydrobenzo[d][1,2,3]diazaborininyl) substituted on the boron by —OH and optionally substituted by one or more groups independently selected from F, Cl, methyl, difluoromethyl, trifluoromethyl, —OH and -OMe;
   X¹ represents C(R²);
   X² represents C(R³);
   R² and R³ independently represent H or R^{a};
   R⁴ represents —NO₂;
   R⁵ and R⁶ independently represent H, halo or R^{c};
   R⁷ represents H; or
   R⁵ and R⁶, or R⁶ and R⁷ (e.g. R⁵ and R⁶) are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F and R^{c};
   each R^{a} and R^{c} independently represents C₁₋₆alkyl optionally substituted by one or more F;
   each R^{a1} independently represents H or C₁₋₃alkyl optionally substituted by one or more F; or
   two R^{a1} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O.

One embodiment refers to compounds of formula I according to the first aspect of the invention, wherein A represents -L¹-L²-L³-A¹;

-L¹-L²-L³- represents:
(i) a single bond;
(ii) —CH₂—;
(iii) —CH₂CH₂— or —CH(Me)-; or
(iv) —C(O)— or —S(O)₂—;
   A¹ represents phenyl substituted by —BF₃K or —B(OR^{a1})₂, (e.g. —B(OR^{a1})₂) and optionally substituted by F, Cl, methyl, —OH or -OMe;
   X¹ represents C(R²);
   X² represents C(R³);
   R² and R³ independently represent H or C₁₋₃alkyl optionally substituted by one or more F (e.g. methyl or trifluoromethyl);
   R⁴ represents —NO₂;
   R⁵ and R⁶ independently represent H, halo or R^{c};
   R⁷ represents H;
   each R^{c} independently represents C₁₋₃alkyl optionally substituted by one or more F;
   each R^{a1} independently represents H or C₁₋₃alkyl optionally substituted by one or more F; or
   two R^{a} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O.

Another embodiment refers to compounds of formula I according to the first aspect of the invention, wherein A represents —CH₂-A¹;

A¹ represents phenyl substituted in the meta- or para-position (e.g. in the para-position) by —B(OH)₂, —B(OMe)₂, 4,4,5,5-tetramethyl-1,3,2-dioxaborolan-2-yl or 6-methyl-1,3,6,2-dioxazaborocane-4,8-dion-2-yl;
X¹ represents C(R²);
X² represents C(R³);
R² and R³ independently represent H or C₁₋₃alkyl optionally substituted by one or more F (e.g. methyl or trifluoromethyl);
R⁴ represents —NO₂;
R⁵ and R⁶ independently represent H, halo or R^{c};
R⁷ represents H; and
each R^{c} independently represents C₁₋₄alkyl optionally substituted by one or more F.

A further embodiment refers to compounds of formula I according to the first aspect of the invention, wherein A represents -L¹-L²-L³-A¹;

each one of L¹ and L³ independently represents a single bond or C₁₋₃alkylene;
L² represents a single bond, —C(O)— or —S(O)₂—;
A¹ represents phenyl optionally substituted by one to three groups independently selected from Y¹, or heteroaryl optionally substituted by one to three groups independently selected from Y²;
X¹ represents C(R²);
X² represents C(R³);
R² and R³ independently represent H or R^{a};
R⁴ represents H;
R⁵ and R⁶ independently represent H, halo or R^{c}; or
R⁴ and R⁵, or R⁵ and R⁶ (eg. R⁵ and R⁶) are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F and R^{c};
R⁷ represents —NO₂;
each R^{a} and R^{c} independently represents C₁₋₄alkyl optionally substituted by one or more F;
each R^{d}, R^{e}, R^{f}, R^{g}, R^{h}, Rⁱ, R^{j}, R^{k}, R^{l}, R^{m}, Rⁿ, R^{o} and R^{r} independently represents H or C₁₋₆alkyl optionally substituted by one or more F; or
R^{d} and R^{e} and/or R^{o} and R^{P} are linked together to form, along with the nitrogen atom to which they are attached, a 3- to 6-membered ring, which ring optionally contains one further heteroatom and which ring optionally is substituted by one or more F, one or more C₁₋₃alkyl each optionally and independently substituted by one or more F, or ═O;
each Y¹ and Y² independently represent halo, R^{b1}, —CN, —C(Q²)R^{c1}, —C(O)OR^{d1}, —C(O)N(R^{e1})R^{f1}, —N(Rⁱ¹)C(O)R^{j1}, —N(R^{p1})S(O)₂R^{q1}, —OR^{u1}, —OC(O)R^{v1}, —S(O)₂R^{ab1} or heteroaryl optionally substituted by one or more groups independently selected from Z³;
each Z³ independently represents halo (e.g. F or Cl) or C₁₋₃alkyl optionally substituted (e.g unsubstituted, e.g. methyl) by one or more F (e.g. difluoromethyl or trifluoromethyl);
Q² represents ═O or ═N(OH);
each R^{b1}, R^{q1} and R^{ab1} independently represents C₁₋₃alkyl optionally substituted by one or more F; and
each R^{c1}, R^{d1}, R^{e1}, R^{f1}, Rⁱ¹, R^{j1}, R^{p1}, R^{r1}, R^{u1} or R^{v1} independently represents H or C₁₋₃alkyl optionally substituted by one or more F.

One embodiment refers to compounds of formula I according to the first aspect of the invention, wherein A represents -L¹-L²-L³-A¹;

-L¹-L²-L³- represents:
(i) a single bond;
(ii) —CH₂—;
(iii) —CH₂CH₂— or —CH(Me)-; or
(iv) —C(O)— or —S(O)₂—;
   A¹ represents phenyl optionally substituted (e.g. in the para position) by one or two (e.g. one) groups independently selected from F, Cl, C₁₋₃alkyl optionally substituted by one or more F and —OC₁₋₃alkyl optionally substituted by one or more F (e.g. -OMe);
   X¹ represents C(R²);
   X² represents C(R³);
   R² and R³ independently represent H or C₁₋₃alkyl optionally substituted by one or more F (e.g. methyl or trifluoromethyl);
   R⁴ represents H;
   R⁵ and R⁶ independently represent H, halo or R^{c};
   R⁷ represents —NO₂; and
   each R^{c} independently represents C₁₋₃alkyl optionally substituted by one or more F.

Another embodiment refers to compounds of formula I according to the first aspect of the invention, wherein A represents -L¹-L²-L³-A¹;

each one of L¹ and L³ independently represents a single bond or C₁₋₃alkylene;
L² represents a single bond, —C(O)— or —S(O)₂—;
A¹ represents:
(i) phenyl substituted by —BF₃K or —B(OR^{a1})₂, (e.g. —B(OR^{a1})₂) and optionally substituted by F, Cl, methyl, difluoromethyl, trifluoromethyl, —OH or -OMe;
(ii) monocyclic heteroaryl substituted by —BF₃K or —B(OR^{a1})₂, (e.g. —B(OR^{a1})₂) and optionally substituted by F, Cl, methyl, difluoromethyl, trifluoromethyl, —OH or -OMe; or
(iii) bicyclic, boron containing, partly aromatic heteroaryl, (e.g. 1,3-dihydrobenzo[c][1,2]oxaborolyl or 1,2-dihydrobenzo[d][1,2,3]diazaborininyl) substituted on the boron by —OH and optionally substituted by one or more groups independently selected from F, Cl, methyl, difluoromethyl, trifluoromethyl, —OH and -OMe;
   X¹ represents C(R²);
   X² represents C(R³);
   R² and R³ independently represent H or R^{a};
   R⁴ represents H;
   R⁵ and R⁶ independently represent H, halo or R^{c}; or
   R⁴ and R⁵, or R⁵ and R⁶ (e.g., R⁵ and R⁶) are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F and R^{c};
   R⁷ represents —NO₂;
   each R^{a} and R^{c} independently represents C₁₋₆alkyl optionally substituted by one or more F;
   each R^{a1} independently represents H or C₁₋₃alkyl optionally substituted by one or more F; or
   two R^{a1} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O.

A further embodiment refers to compounds of formula I according to the first aspect of the invention, wherein A represents-L-L²-L³-A¹;

-L¹-L²-L³- represents:
(i) a single bond;
(ii) —CH₂—;
(iii) —CH₂CH₂— or —CH(Me)-; or
(iv) —C(O)— or —S(O)₂—;
   A¹ represents phenyl substituted by —BF₃K or —B(OR^{a1})₂, (e.g. —B(OR^{a1})₂) and optionally substituted by F, Cl, methyl, —OH or -OMe;
   X¹ represents C(R²);
   X² represents C(R³);
   R² and R³ independently represent H or C₁₋₃alkyl optionally substituted by one or more F (e.g. methyl or trifluoromethyl);
   R⁴ represents H;
   R⁵ and R⁶ independently represent H, halo or R^{c};
   R⁷ represents —NO₂;
   each R^{c} independently represents C₁₋₃alkyl optionally substituted by one or more F;
   each R^{a1} independently represents H or C₁₋₃alkyl optionally substituted by one or more F; or
   two R^{a1} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O.

One embodiment refers to compounds of formula I according to the first aspect of the invention, wherein A represents —CH₂-A¹;

A¹ represents phenyl substituted in the meta- or para-position (e.g. in the para-position) by —B(OH)₂, —B(OMe)₂, 4,4,5,5-tetramethyl-1,3,2-dioxaborolan-2-yl or 6-methyl-1,3,6,2-dioxazaborocane-4,8-dion-2-yl;
X¹ represents C(R²);
X² represents C(R³);
R² and R³ independently represent H or C₁₋₃alkyl optionally substituted by one or more F (e.g. methyl or trifluoromethyl);
R⁴ represents H;
R⁵ and R⁶ independently represent H, halo or R^{c};
R⁷ represents —NO₂; and
each R^{c} independently represents C₁₋₄alkyl optionally substituted by one or more F.

Another embodiment refers to compounds of formula I according to the first aspect of the invention, wherein A represents -L¹-L²-L³-A¹;

each one of L¹ and L³ independently represents a single bond or C₁₋₃alkylene;
L² represents a single bond, —C(O)—, —S(O)₂— or —C(O)N(H)—;
A¹ represents:
(i) phenyl substituted by —BF₃K or —B(OR^{a1})₂, (e.g. —B(OR^{a1})₂) and optionally substituted by F, Cl, methyl, difluoromethyl, trifluoromethyl, —OH or -OMe;
(ii) monocyclic heteroaryl substituted by —BF₃K, or —B(OR^{a1})₂, (e.g. —B(OR^{a1})₂) and optionally substituted by F, Cl, methyl, difluoromethyl, trifluoromethyl, —OH or -OMe; or
(iii) bicyclic, boron containing, partly aromatic heteroaryl, (e.g. 1,3-dihydrobenzo[c][1,2]oxaborolyl or 1,2-dihydrobenzo[d][1,2,3]diazaborininyl) substituted on the boron by —OH and optionally substituted by one or more groups independently selected from F, Cl, methyl, difluoromethyl, trifluoromethyl, —OH and -OMe;
   X¹ represents N;
   X² represents C(R³);
   R³ represents H or R^{a};
   R⁴ represents —NO₂;
   R⁵ and R⁶ independently represent H, halo or R^{c};
   R⁷ represents H; or
   R⁵ and R⁶, or R⁶ and R⁷ (e.g. R⁵ and R⁶) are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F and R^{c};
   each R^{a} and R^{c} independently represents C₁₋₄alkyl optionally substituted by one or more F;
   each R^{a1} independently represents H or C₁₋₃alkyl optionally substituted by one or more F; or
   two R^{a1} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O.

A further embodiment refers to compounds of formula I according to the first aspect of the invention, wherein A represents -L¹-L²-L³-A¹;

-L¹-L²-L³- represents:
(i) a single bond;
(ii) —CH₂—;
(iii) —CH₂CH₂— or —CH(Me)-; or
(iv) —C(O)— or —S(O)₂—;
   A¹ represents phenyl substituted by —BF₃K, or —B(OR^{a1})₂, (e.g. —B(OR^{a1})₂) and optionally substituted by F, Cl, methyl, —OH or -OMe;
   X¹ represents N;
   X² represents C(R³);
   R³ represents H or C₁₋₃alkyl optionally substituted by one or more F (e.g. methyl or trifluoromethyl);
   R⁴ represents —NO₂;
   R⁵ and R⁶ independently represent H, halo or R^{c}; or
   R⁵ and R⁶, are linked together to form, along with the carbon atoms to which they are attached, a 5- or 6-membered ring, which ring optionally is substituted by one or more C₁₋₃alkyl optionally substituted by one or more F (e.g. methyl);
   R⁷ represents H; and
   each R^{c} independently represents C₁₋₃alkyl optionally substituted by one or more F.

One embodiment refers to compounds of formula I according to the first aspect of the invention, wherein A represents —CH₂-A¹;

A¹ represents phenyl substituted (e.g. in the meta- or para-position (e.g. in the para-position) by —B(OH)₂, —B(OMe)₂, 4,4,5,5-tetramethyl-1,3,2-dioxaborolan-2-yl or 6-methyl-1,3,6,2-dioxazaborocane-4,8-dion-2-yl;
X¹ represents N;
X² represents C(R³);
R³ represents H or C₁₋₃alkyl optionally substituted by one or more F (e.g. methyl or trifluoromethyl);
R⁴ represents —NO₂;
R⁵ and R⁶ independently represent H, halo or R^{c}; or
R⁵ and R⁶ are linked together to form, along with the carbon atoms to which they are attached, a 5- or 6-membered ring, which ring optionally is substituted by one or more C₁₋₃alkyl optionally substituted by one or more F (e.g. methyl); and
R⁷ represents H.

Another embodiment refers to compounds of formula I according to the first aspect of the invention, wherein A represents -L¹-L²-L³-A¹;

each one of L¹ and L³ independently represents a single bond or C₁₋₃alkylene;
L² represents a single bond, —C(O)— or —S(O)₂—;
A¹ represents:
(i) phenyl substituted by —BF₃K or —B(OR^{a1})₂, (e.g. —B(OR^{a1})₂) and optionally substituted by F, Cl, methyl, difluoromethyl, trifluoromethyl, —OH or -OMe;
(ii) monocyclic heteroaryl substituted by —BF₃K, or —B(OR^{a1})₂, (e.g. —B(OR^{a1})₂) and optionally substituted by F, Cl, methyl, difluoromethyl, trifluoromethyl, —OH or -OMe; or
(iii) bicyclic, boron containing, partly aromatic heteroaryl, (e.g. 1,3-dihydrobenzo[c][1,2]oxaborolyl or 1,2-dihydrobenzo[d][1,2,3]diazaborininyl) substituted on the boron by —OH and optionally substituted by one or more groups independently selected from F, Cl, methyl, difluoromethyl, trifluoromethyl, —OH and -OMe;
   X¹ represents N;
   X² represents C(R³);
   R³ represents H or R^{a};
   R⁴ represents H;
   R⁵ and R⁶ independently represent H, halo or R^{c}; or
   R⁴ and R⁵, or R⁵ and R⁶ (e.g. R⁵ and R⁶) are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F and R^{c};
   R⁷ represents —NO₂;
   each R^{a} and R^{c} independently represents C₁₋₄alkyl optionally substituted by one or more F;
   each R^{a1} independently represents H or C₁₋₃alkyl optionally substituted by one or more F; or
   two R^{a1} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O.

A further embodiment refers to compounds of formula I according to the first aspect of the invention, wherein A represents -L¹-L²-L³-A¹;

-L¹-L²-L³- represents:
(i) a single bond;
(ii) —CH₂—;
(iii) —CH₂CH₂- or —CH(Me)-; or
(iv) —C(O)— or —S(O)₂—;
   A¹ represents phenyl substituted by —BF₃K, or —B(OR^{a1})₂, (e.g. —B(OR^{a1})₂) and optionally substituted by F, Cl, methyl, —OH or -OMe;
   X¹ represents N;
   X² represents C(R³);
   R³ represents H or C₁₋₃alkyl optionally substituted by one or more F (e.g. methyl or trifluoromethyl);
   R⁴ represents H;
   R⁵ and R⁶ independently represent H, halo or R^{c}; or
   R⁵ and R⁶, are linked together to form, along with the carbon atoms to which they are attached, a 5- or 6-membered ring, which ring optionally is substituted by one or more C₁₋₃alkyl optionally substituted by one or more F (e.g. methyl);
   R⁷ represents NO₂; and
   each R^{c} independently represents C₁₋₃alkyl optionally substituted by one or more F.

One embodiment refers to compounds of formula I according to the first aspect of the invention, wherein A represents —CH₂-A¹;

A¹ represents phenyl substituted in the meta- or para-position (e.g. in the para-position) by —B(OH)₂, —B(OMe)₂, 4,4,5,5-tetramethyl-1,3,2-dioxaborolan-2-yl or 6-methyl-1,3,6,2-dioxazaborocane-4,8-dion-2-yl;
X¹ represents N;
X² represents C(R³);
R³ represent H or C₁₋₃alkyl optionally substituted by one or more F (e.g. methyl or trifluoromethyl);
R⁴ represents H;
R⁵ and R⁶ independently represent H, halo or R^{c}; or
R⁵ and R⁶, are linked together to form, along with the carbon atoms to which they are attached, a 5- or 6-membered ring, which ring optionally is substituted by one or more C₁₋₃alkyl optionally substituted by one or more F (e.g. methyl).
R⁷ represents —NO₂.

One embodiment refers to compounds according to the first aspect of the invention selected from the group comprising
1-(4-methoxybenzyl)-2-methyl-4-nitro-1H-benzo[d]imidazole (Exemplified compound 2.2.1);
1-(4-methoxybenzyl)-4-nitro-2-phenyl-1H-benzo[d]imidazole (2.2.2);
2-benzyl-1-(4-methoxybenzyl)-4-nitro-1H-benzo[d]imidazole (2.2.3);
1-(4-methoxybenzyl)-4-nitro-2-(trifluoromethyl)-1H-benzo[d]imidazole (2.2.4);
2-methoxy-1-(4-methoxybenzyl)-4-nitro-1H-benzo[d]imidazole (2.2.5);
1-(4-methoxybenzyl)-2-(methoxymethyl)-4-nitro-1H-benzo[d]imidazole (2.2.6);
2-isopropyl-1-(4-methoxybenzyl)-4-nitro-1H-benzo[d]imidazole (2.2.7);
(4-((4-nitro-2-(trifluoromethyl)-1H-benzo[d]imidazol-1-yl)methyl)phenyl)boronic acid (2.2.8);
5,6-difluoro-1-(4-methoxybenzyl)-2-methyl-4-nitro-1H-benzo[d]imidazole (2.2.9);
1-benzyl-5,6-difluoro-2-methyl-4-nitro-1H-benzo[d]imidazole (2.2.10);
4-((5,6-difluoro-2-methyl-4-nitro-1H-benzo[d]imidazol-1-yl)methyl)benzonitrile (2.2.11);
5,6-difluoro-1-(4-fluorobenzyl)-2-methyl-4-nitro-1H-benzo[d]imidazole (2.2.12);
1-(4-(1H-1,2,4-triazol-1-yl)benzyl)-5,6-difluoro-2-methyl-4-nitro-1H-benzo[d]imidazole (2.2.13);
5,6-difluoro-2-methyl-4-nitro-1-(1-phenylethyl)-1H-benzo[d]imidazole (2.2.14);
(4-((5,6-difluoro-2-methyl-4-nitro-1H-benzo[d]imidazol-1-yl)methyl)phenyl)boronic acid (2.2.15);
5,6-dichloro-2-methyl-4-nitro-1-phenyl-1H-benzo[d]imidazole (2.2.16);
5,6-dichloro-2-methyl-4-nitro-1-(phenylsulfonyl)-1H-benzo[d]imidazole (2.2.17);
(5,6-dichloro-2-methyl-4-nitro-1H-benzo[d]imidazol-1-yl)(phenyl)methanone (2.2.18);
1-benzyl-5,6-dichloro-2-methyl-4-nitro-1H-benzo[d]imidazole (2.2.19);
5,6-dichloro-1-(4-methoxybenzyl)-2-methyl-4-nitro-1H-benzo[d]imidazole (2.2.20);
5,6-dichloro-1-(3-methoxybenzyl)-2-methyl-4-nitro-1H-benzo[d]imidazole (2.2.21);
4-((5,6-dichloro-2-methyl-4-nitro-1H-benzo[d]imidazol-1-yl)methyl)phenyl acetate (2.2.22);
N-(4-((5,6-dichloro-2-methyl-4-nitro-1H-benzo[d]imidazol-1-yl)methyl)phenyl)acetamide (2.2.23);
4-((5,6-dichloro-2-methyl-4-nitro-1H-benzo[d]imidazol-1-yl)methyl)aniline (2.2.24);
4-((5,6-dichloro-2-methyl-4-nitro-1H-benzo[d]imidazol-1-yl)methyl)phenol (2.2.25);
5,6-dichloro-2-methyl-1-(4-methylbenzyl)-4-nitro-1H-benzo[d]imidazole (2.2.26);
4-((5,6-dichloro-2-methyl-4-nitro-1H-benzo[d]imidazol-1-yl)methyl)benzaldehyde (2.2.27);
4-((5,6-dichloro-2-methyl-4-nitro-1H-benzo[d]imidazol-1-yl)methyl)benzaldehyde oxime (2.2.28);
1-(4-((5,6-dichloro-2-methyl-4-nitro-1H-benzo[d]imidazol-1-yl)methyl)phenyl)prop-2-en-1-ol (2.2.29);
1-(4-((5,6-dichloro-2-methyl-4-nitro-1H-benzo[d]imidazol-1-yl)methyl)phenyl)prop-2-en-1-one (2.2.30);
2-bromo-5-((5,6-dichloro-2-methyl-4-nitro-1H-benzo[d]imidazol-1-yl)methyl)benzaldehyde (2.2.31);
N-(4-((5,6-dichloro-2-methyl-4-nitro-1H-benzo[d]imidazol-1-yl)methyl)phenyl)acrylamide (2.2.32);
5,6-dichloro-1-(2-(3,5-dimethyl-1H-pyrazol-4-yl)ethyl)-2-methyl-4-nitro-1H-benzo[d]imidazole (2.2.33);
1-(4-(1H-pyrazol-1-yl)benzyl)-5,6-dichloro-2-methyl-4-nitro-1H-benzo[d]imidazole (2.2.34);
1-(4-(1H-1,2,4-triazol-1-yl)benzyl)-5,6-dichloro-2-methyl-4-nitro-1H-benzo[d]imidazole (2.2.35);
1-(4-(1H-pyrrol-1-yl)benzyl)-5,6-dichloro-2-methyl-4-nitro-1H-benzo[d]imidazole (2.2.36);
4-((5,6-dichloro-2-methyl-4-nitro-1H-benzo[d]imidazol-1-yl)methyl)benzoic acid (2.2.37);
methyl 4-((5,6-dichloro-2-methyl-4-nitro-1H-benzo[d]imidazol-1-yl)methyl)benzoate (2.2.38);
4-((5,6-dichloro-2-methyl-4-nitro-1H-benzo[d]imidazol-1-yl)methyl)benzamide (2.2.39);
4-((5,6-dichloro-2-methyl-4-nitro-1H-benzo[d]imidazol-1-yl)methyl)benzonitrile (2.2.40);
5,6-dichloro-2-methyl-1-(4-(methylsulfonyl)benzyl)-4-nitro-1H-benzo[d]imidazole (2.2.41);
4-((5,6-dichloro-2-methyl-4-nitro-1H-benzo[d]imidazol-1-yl)methyl)-2-methylthiazole (2.2.42);
5,6-dichloro-1-((6-chloropyridin-3-yl)methyl)-2-methyl-4-nitro-1H-benzo[d]imidazole (2.2.43);
(4-((5,6-dichloro-2-methyl-4-nitro-1H-benzo[d]imidazol-1-yl)methyl)phenyl)boronic acid (2.2.44);
(4-((5,6-dichloro-2-cyclopropyl-4-nitro-1H-benzo[d]imidazol-1-yl)methyl)phenyl)boronic acid (2.2.45);
(2-((5,6-dichloro-2-methyl-4-nitro-1H-benzo[d]imidazol-1-yl)methyl)phenyl)boronic acid (2.2.46);
(3-((5,6-dichloro-2-methyl-4-nitro-1H-benzo[d]imidazol-1-yl)methyl)phenyl)boronic acid (2.2.47);
5,6-dichloro-2-methyl-4-nitro-1-(4-(trifluoro-l4-boranyl)benzyl)-1H-benzo[d]imidazole (2.2.48), or potassium salt thereof;
5,6-dichloro-2-methyl-4-nitro-1-(4-(4,4,5,5-tetramethyl-1,3,2-dioxaborolan-2-yl)benzyl)-1H-benzo[d]imidazole (2.2.49);
2-(4-((5,6-dichloro-2-methyl-4-nitro-1H-benzo[d]imidazol-1-yl)methyl)phenyl)-6-methyl-1,3,6,2-dioxazaborocane-4,8-dione (2.2.50);
(4-(2-(5,6-dichloro-2-methyl-4-nitro-1H-benzo[d]imidazol-1-yl)ethyl)phenyl)boronic acid (2.2.51);
2,5,6-trimethyl-4-nitro-1-(3,4,5-trimethoxybenzyl)-1H-benzo[d]imidazole (2.2.52);
1-((6-chlorobenzo[d][1,3]dioxol-5-yl)methyl)-2,5,6-trimethyl-4-nitro-1H-benzo[d]imidazole (2.2.53);
1-(4-methoxybenzyl)-2,5,6-trimethyl-4-nitro-1H-benzo[d]imidazole (2.2.54);
1-(4-chlorobenzyl)-2,5,6-trimethyl-4-nitro-1H-benzo[d]imidazole (2.2.55);
1-(4-fluorobenzyl)-2,5,6-trimethyl-4-nitro-1H-benzo[d]imidazole (2.2.56);
2,5,6-trimethyl-4-nitro-1-(4-(trifluoromethoxy)benzyl)-1H-benzo[d]imidazole (2.2.57);
1-(4-methoxybenzyl)-5,6-dimethyl-4-nitro-2-(trifluoromethyl)-1H-benzo[d]imidazole (2.2.58);
2,5,6-trimethyl-4-nitro-1-(1-phenylethyl)-1H-benzo[d]imidazole (2.2.59);
(4-((2,5,6-trimethyl-4-nitro-1H-benzo[d]imidazol-1-yl)methyl)phenyl)boronic acid (2.2.60);
(4-((5,6-dimethyl-4-nitro-2-(trifluoromethyl)-1H-benzo[d]imidazol-1-yl)methyl)phenyl)boronic acid (2.2.61);
1-(4-methoxybenzyl)-4-nitro-1H-benzo[d]imidazole (2.2.62);
(4-((4-nitro-1H-benzo[d]imidazol-1-yl)methyl)phenyl)boronic acid (2.2.63);
(4-((5,5,7,7-tetramethyl-4-nitro-6,7-dihydroindeno[5,6-d]imidazol-1 (5H)-yl)methyl)phenyl)boronic acid (2.2.64);
2-benzyl-1-(4-methoxybenzyl)-7-nitro-1H-benzo[d]imidazole (2.3.1);
2-methoxy-1-(4-methoxybenzyl)-7-nitro-1H-benzo[d]imidazole (2.3.2);
1-(4-methoxybenzyl)-7-nitro-2-phenyl-1H-benzo[d]imidazole (2.3.3) and
(4-((5,6-difluoro-2-methyl-7-nitro-1H-benzo[d]imidazol-1-yl)methyl)phenyl)boronic acid (2.3.4),
   or a pharmaceutically acceptable salt thereof.

In an embodiment of the invention, the compound according to the invention is selected from the compounds 2.2.1 to 2.2.64 and 2.3.1 to 2.3.4.

Compounds have been named using the software ChemBioDraw v. 13.0. In case of doubt or seemingly inconsistence, the formula structure of the compounds prevail.

In a second aspect of the invention there is provided a compound of formula I, or a pharmaceutically acceptable salt thereof,

wherein:
A represents -L¹-L²-L³-A¹;
A¹ represents:
(i) aryl substituted by —BF₃M or —B(OR^{a1})₂, and optionally substituted by one or more groups independently selected from Y¹;
(ii) heteroaryl substituted by —BF₃M or —B(OR^{a1})₂, and optionally substituted by one or more groups independently selected from Y²; or
(iii) bicyclic, boron containing, partly aromatic heteroaryl substituted on the boron by —OH and optionally substituted by one or more groups independently selected from Y³;
   each one of L¹ and L³ independently represents a single bond or C₁₋₃alkylene optionally substituted by one or more halo;
   L² represents a single bond, —C(Q)-, —N(R¹)—, —O—, —S(O)ₙ—, —C(Q)N(R¹)—, —N(R¹)C(Q)-, —C(O)O—, —OC(O)—, —S(O)ₙN(R¹)— or —N(R¹)S(O)ₙ—;
   X¹ represents C(R²);
   X² represents N;
   each R¹ independently represents H or C₁₋₆alkyl optionally substituted by one or more halo;
   R² represents H, R^{a} or —OR^{b};
   R⁴ and R⁷ independently represent H, halo, —CN, R^{c}, —C(H)(CF₃)OH, —C(CF₃)₂OH, —C(OH)₂CF₃, —N₃, —NO₂, —N(R^{d})R^{e}, —N(R^{f})C(Q¹)R^{g}, —N(R^{h})S(O)ₙRⁱ, —OR^{j}, —SR^{k} or —C(O)R⁸;
   R⁵ and R⁶ independently represent H, halo, —CN, R^{c}, —N₃, —NO₂, —OR^{j} or —SR^{k}; or
   R⁴ and R⁵, R⁵ and R⁶ and/or R⁶ and R⁷ are linked together to form, along with the carbon atoms to which they are attached, a 5- or 6-membered ring, which ring optionally contains one to three heteroatoms and/or one or two further double bonds, and which ring optionally is substituted by one or more groups independently selected from halo, —OR^{j}, C₁₋₃alkyl optionally substituted by one or more halo, and Q¹;
   each R⁸ independently represents —OR^{l}, —N(H)R^{m}, —N(H)C(Q¹)Rⁿ, —N(H)C(Q¹)N(R^{o})R^{P}, —N(H)OH or —N(H)S(O)ₙR^{q};
   Q represents ═O or ═S;
   Q¹ represents ═O, ═NR^{r} or ═S;
   R^{a} represents C₁₋₆alkyl optionally substituted by one or more groups independently selected from D¹, aryl optionally substituted by one or more groups independently selected from D² or heteroaryl optionally substituted by one or more groups independently selected from D³;
   each R^{c} and R^{q} independently represents C₁₋₆alkyl optionally substituted by one or more halo;
   each R^{b}, R^{d}, R^{e}, R^{f}, R^{g}, R^{h}, Rⁱ, R^{j}, R^{k}, R^{l}, R^{m}, Rⁿ, R^{o}, R^{P} and R^{r} independently represents H or C₁₋₆alkyl optionally substituted by one or more halo; or
   R^{d} and R^{e} and/or R^{o} and R^{p} are linked together to form, along with the nitrogen atom to which they are attached, a 3- to 6-membered ring, which ring optionally contains one further heteroatom and which ring optionally is substituted by one or more halo, one or more C₁₋₃alkyl each optionally and independently substituted by one or more F, or ═O;
   D¹ represents halo, —OC₁₋₆alkyl optionally substituted by one or more halo, aryl optionally substituted by one or more groups independently selected from D² or heteroaryl optionally substituted by one or more groups independently selected from D³;
   each D² and D³ independently represents halo, C₁₋₆alkyl optionally substituted by one or more halo or —OC₁₋₆alkyl optionally substituted by one or more halo;
   each Y¹, Y² and Y³ independently represents halo, R^{b1}, —CN, or —OR^{u1};
   M represents a cation selected from (R^{M})₄N⁺, Li⁺, Na⁺, K⁺, Rb⁺ or Cs⁺;
   each R^{M} independently represents C₁₋₁₂alkyl optionally substituted by one or more D⁴;
   each R^{b1} independently represents C₁₋₆ alkyl optionally substituted by one or more groups independently selected from D⁴;
   each R^{a1} and R^{u1} independently represents H or C₁₋₆ alkyl optionally substituted by one or more groups independently selected from D⁴; or
   two R^{a1} are linked together to form, along with the boron, and the oxygen atoms to which they are attached, a 5- to 8-membered heterocyclic ring, which ring optionally contains one or more further heteroatoms and which ring optionally and independently is substituted by one or more groups independently selected from halo, C₁₋₃alkyl optionally substituted by one or more halo, and =0;
   each D⁴ independently represents halo, —OH or —OC₁₋₆alkyl optionally substituted by one or more halo;
   each n independently represents 1 or 2;
   provided that at least one of R⁴ and R⁷ represent —C(H)(CF₃)OH, —C(CF₃)₂OH, —C(OH)₂CF₃, —NO₂ or —C(O)R⁸; and
   provided that formula I does not represent
1-(4-boronobenzyl)-2-ethoxy-1H-benzo[d]imidazole-7-carboxylic acid,
ethyl 1-(4-(5,5-dimethyl-1,3,2-dioxaborinan-2-yl)benzyl)-2-ethoxy-1H-benzo[d]imidazole-7-carboxylate,
methyl 1-(3-(4,4,5,5-tetramethyl-1,3,2-dioxaborolan-2-yl)benzyl)-1H-indole-7-carboxylate, or
methyl 1-(4-(4,4,5,5-tetramethyl-1,3,2-dioxaborolan-2-yl)benzyl)-1H-indole-7-carboxylate.

These compounds are referred to herein as compounds of the invention, or compounds of the invention according to the second aspect of the invention.

One embodiment refers to compounds of formula I according to the second aspect of the invention, wherein A represents -L¹-L²-L³-A¹;

each one of L¹ and L³ independently represents a single bond or C₁₋₃alkylene;
L² represents a single bond, —C(O)—, —S(O)₂— or —C(O)N(H)—;
A¹ represents:
(i) phenyl substituted by —BF₃K or —B(OR^{a1})₂ (e.g. —B(OR^{a1})₂) and optionally substituted by F, Cl, methyl, difluoromethyl, trifluoromethyl, —OH or -OMe;
(ii) monocyclic heteroaryl substituted by —BF₃K or —B(OR^{a1})₂ (e.g. —B(OR^{a1})₂) and optionally substituted by F, Cl, methyl, difluoromethyl, trifluoromethyl, —OH or -OMe; or
(iii) bicyclic, boron containing, partly aromatic heteroaryl, (e.g. 1,3-dihydrobenzo[c][1,2]oxaborolyl or 1,2-dihydrobenzo[d][1,2,3]diazaborininyl) substituted on the boron by —OH and optionally substituted by one or more groups independently selected from F, Cl, methyl, difluoromethyl, trifluoromethyl, —OH and -OMe;
   X¹ represents C(R²);
   X² represents N;
   R² represents R^{a} or —OR^{b};
   R⁴ represents —NO₂ or —C(O)R⁸;
   R⁵ and R⁶ independently represent H, halo or R^{c};
   R⁷ represents H; or
   R⁵ and R⁶, or R⁶ and R⁷ (e.g. R⁵ and R⁶) are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F and R^{c};
   R⁸ represents —OR^{l}, —N(H)R^{m}, —N(H)C(O)Rⁿ, —N(H)C(O)N(R^{o})R^{P}, —N(H)OH or —N(H)S(O)₂R^{q};
   R^{a} represents:
(i) C₁₋₃alkyl optionally substituted by one to three F (e.g. methyl, ethyl, propyl, isopropyl, cyclopropyl, cyclopropylmethyl, difluoromethyl, trifluoromethyl or 2,2,2-trifluoroethyl) or —OC₁₋₃alkyl optionally substituted by one to three F (e.g. methoxymethyl, trifluoromethoxymethyl or ethoxyethyl);
(ii) —C₁₋₃alkylphenyl (e.g. benzyl) optionally substituted (e.g. not substituted) by one or two (e.g. one) groups independently selected from F, Cl, methyl, difluoromethyl, trifluoromethyl, methoxy, difluoromethoxy and trifluoromethoxy; or
(iii) phenyl optionally substituted (e.g. not substituted) by one or two (e.g. one) groups independently selected from F, Cl, methyl, difluoromethyl, trifluoromethyl, methoxy, difluoromethoxy and trifluoromethoxy;
   R^{b} represents C₁₋₂alkyl optionally substituted by one or more F (e.g. methyl, ethyl, difluoromethyl or trifluoromethyl);
   each R^{c} and R^{q} independently represents C₁₋₆alkyl optionally substituted by one or more F;
   each R^{m}, Rⁿ and R^{o} independently represents H or C₁₋₆alkyl optionally substituted by one or more F; or
   R^{o} and R^{P} are linked together to form, along with the nitrogen atom to which they are attached, a 3- to 6-membered ring, which ring optionally contains one further heteroatom and which ring optionally is substituted by one or more F, one or more C₁₋₃alkyl each optionally and independently substituted by one or more F, or ═O;
   each R^{a1} independently represents H or C₁₋₃alkyl optionally substituted by one or more F; or
   two R^{a1} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O.

Another embodiment refers to compounds of formula I according to the second aspect of the invention, wherein A represents -L¹-L²-L³-A¹;

-L¹-L²-L³- represents:
(i) a single bond;
(ii) —CH₂—;
(iii) —CH₂CH₂— or —CH(Me)-; or
(iv) —C(O)— or —S(O)₂—;
   A¹ represents phenyl substituted by —BF₃K or —B(OR^{a1})₂ (e.g. —B(OR^{a1})₂) and optionally substituted by F, Cl, methyl, —OH or -OMe;
   X¹ represents C(R²);
   X² represents N;
   R² represents R^{a} or —OR^{b};
   R⁴ represents —NO₂ or —C(O)R⁸;
   R⁵ and R⁶ independently represent H, halo or R^{c};
   R⁷ represents H; or
   R⁵ and R⁶, or R⁶ and R⁷ (e.g. R⁵ and R⁶) are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F and R^{c};
   R⁸ represents —OR^{l}, —N(H)R^{m}, —N(H)C(O)Rⁿ, —N(H)C(O)N(R^{o})R^{P}, —N(H)OH or —N(H)S(O)₂R^{q};
   R^{a} represents:
(i) C₁₋₃alkyl optionally substituted by one to three F (e.g. methyl, ethyl, propyl, isopropyl, cyclopropyl, cyclopropylmethyl, difluoromethyl, trifluoromethyl or 2,2,2-trifluoroethyl) or —OC₁₋₃alkyl optionally substituted by one to three F (e.g. methoxymethyl, trifluoromethoxymethyl, ethoxyethyl);
(ii) —C₁₋₃alkylphenyl (e.g. benzyl) optionally substituted (e.g. not substituted) by one or two (e.g. one) groups independently selected from F, Cl, methyl, difluoromethyl, trifluoromethyl, methoxy, difluoromethoxy and trifluoromethoxy); or
(iii) phenyl optionally substituted (e.g. not substituted) by one or two (e.g. one) groups independently selected from F, Cl, methyl, difluoromethyl, trifluoromethyl, methoxy, difluoromethoxy and trifluoromethoxy);
   R^{b} represents C₁₋₂alkyl optionally substituted by one or more F (e.g. methyl, ethyl, difluoromethyl or trifluoromethyl);
   each R^{c} and R^{q} independently represents C₁₋₆alkyl optionally substituted by one or more F;
   each R^{l}, R^{m}, Rⁿ and R^{o} independently represents H or C₁₋₆alkyl optionally substituted by one or more F; or
   R^{o} and R^{P} are linked together to form, along with the nitrogen atom to which they are attached, a 3- to 6-membered ring, which ring optionally contains one further heteroatom and which ring optionally is substituted by one or more F, one or more C₁₋₃alkyl each optionally and independently substituted by one or more F, or ═O;
   each R^{a1} independently represents H or C₁₋₃alkyl optionally substituted by one or more F; or
   two R^{a1} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O;

A further embodiment refers to compounds of formula I according to the second aspect of the invention, wherein A represents —CH₂-A¹;

A¹ represents phenyl substituted in the meta- or para-position (e.g. in the para-position) by —B(OH)₂, —B(OMe)₂, 4,4,5,5-tetramethyl-1,3,2-dioxaborolan-2-yl or 6-methyl-1,3,6,2-dioxazaborocane-4,8-dion-2-yl;
X¹ represents C(R²);
X² represents N;
R² represents methyl, isopropyl, cyclopropyl, trifluoromethyl, methoxymethyl, benzyl or phenyl (e.g. methyl, cyclopropyl or trifluoromethyl);
R⁴ represents —NO₂;
R⁵ and R⁶ represent H, or preferably, F, Cl, or methyl, or more preferably at least one of (or preferably both of) R⁵ and R⁶ represent F, Cl, or methyl; and
R⁷ represents H.

One embodiment refers to compounds of formula I according to the second aspect of the invention, wherein A represents -L¹-L²-L³-A¹;

each one of L¹ and L³ independently represents a single bond or C₁₋₃alkylene;
L² represents a single bond, —C(O)—, —S(O)₂— or —C(O)N(H)—;
A¹ represents:
(i) phenyl substituted by —BF₃K or —B(OR^{a1})₂ (e.g. —B(OR^{a1})₂) and optionally substituted by F, Cl, methyl, difluoromethyl, trifluoromethyl, —OH or -OMe;
(ii) monocyclic heteroaryl substituted by —BF₃K or —B(OR^{a1})₂ (e.g. —B(OR^{a1})₂) and optionally substituted by F, Cl, methyl, difluoromethyl, trifluoromethyl, —OH or -OMe; or
(iii) bicyclic, boron containing, partly aromatic heteroaryl, (e.g. 1,3-dihydrobenzo[c][1,2]oxaborolyl or 1,2-dihydrobenzo[d][1,2,3]diazaborininyl) substituted on the boron by —OH and optionally substituted by F, Cl, methyl, difluoromethyl, trifluoromethyl, —OH or -OMe;
   X¹ represents C(R²);
   X² represents N;
   R² represents R^{a} or —OR^{b};
   R⁴ represents H;
   R⁵ and R⁶ independently represent H, halo or R^{c};
   R⁷ represents —NO₂ or —C(O)R⁸; or
   R⁵ and R⁶, or R⁶ and R⁷ (e.g. R⁵ and R⁶) are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F and R^{c};
   R⁸ represents —OR^{l}, —N(H)R^{m}, —N(H)C(O)Rⁿ, —N(H)C(O)N(R^{o})R^{P}, —N(H)OH or —N(H)S(O)₂R^{q};
   R^{a} represents:
(i) C₁₋₃alkyl optionally substituted by one to three F (e.g. methyl, ethyl, propyl, isopropyl, cyclopropyl, cyclopropylmethyl, difluoromethyl, trifluoromethyl or 2,2,2-trifluoroethyl) or —OC₁₋₃alkyl optionally substituted by one to three F (e.g. methoxymethyl, trifluoromethoxymethyl or ethoxyethyl);
(ii) —C₁₋₃alkylphenyl (e.g. benzyl) optionally substituted (e.g. not substituted) by one or two (e.g. one) groups independently selected from F, Cl, methyl, difluoromethyl, trifluoromethyl, methoxy, difluoromethoxy and trifluoromethoxy); or
(iii) phenyl optionally substituted by (e.g. not substituted) by one or two (e.g. one) groups independently selected from F, Cl, methyl, difluoromethyl, trifluoromethyl, methoxy, difluoromethoxy and trifluoromethoxy;
   R^{b} represents C₁₋₂alkyl optionally substituted by one or more F (e.g. methyl, ethyl, difluoromethyl or trifluoromethyl);
   each R^{c} and R^{q} independently represents C₁₋₆alkyl optionally substituted by one or more F;
   each R^{l}, R^{m}, Rⁿ and R^{o} independently represents H or C₁₋₆alkyl optionally substituted by one or more F; or
   R^{o} and R^{p} are linked together to form, along with the nitrogen atom to which they are attached, a 3- to 6-membered ring, which ring optionally contains one further heteroatom and which ring optionally is substituted by one or more F, one or more C₁₋₃alkyl each optionally and independently substituted by one or more F, or ═O;
   each R^{a1} independently represents H or C₁₋₃alkyl optionally substituted by one or more F; or
   two R^{a1} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O.

Another embodiment refers to compounds of formula I according to the second aspect of the invention, wherein A represents -L¹-L²-L³-A¹;

-L¹-L²-L³- represents —C(O)— or —S(O)₂—, or preferably, a single bond, —CH₂CH₂— or —CH(Me)-, or more preferably, —CH₂—;
A¹ represents phenyl substituted by —BF₃K or —B(OR^{a1})₂ (e.g. —B(OR^{a1})₂) and optionally substituted by F, Cl, methyl, —OH or -OMe;
X¹ represents C(R²);
X² represents N;
R² represents R^{a} or —OR^{b};
R⁴ represents H;
R⁵ and R⁶ independently represent H, halo or R^{c};
R⁷ represents —NO₂ or —C(O)R⁸; or
R⁴ and R⁵, or R⁵ and R⁶ (e.g. R⁵ and R⁶) are linked together to form, along with the carbon atoms to which they are attached, a 5- or 6-membered ring, which ring optionally contains one to three heteroatoms and/or one or two (e.g. one) further double bonds, and which ring optionally is substituted by one or more groups independently selected from F and R^{c};
R⁸ represents —OR^{l}, —N(H)R^{m}, —N(H)C(O)Rⁿ, —N(H)C(O)N(R^{o})R^{P}, —N(H)OH or —N(H)S(O)₂R^{q};
R^{a} represents:
(i) C₁₋₃alkyl optionally substituted by one to three F (e.g. methyl, ethyl, propyl, isopropyl, cyclopropyl, cyclopropylmethyl, difluoromethyl, trifluoromethyl or 2,2,2-trifluoroethyl) or —OC₁₋₃alkyl optionally substituted by one to three F (e.g. methoxymethyl, trifluoromethoxymethyl or ethoxyethyl);
(ii) C₁₋₃alkylphenyl (e.g. benzyl) optionally substituted (e.g. not substituted) by one or two (e.g. one) groups independently selected from F, Cl, methyl, difluoromethyl, trifluoromethyl, methoxy, difluoromethoxy and trifluoromethoxy; or
(iii) phenyl optionally substituted (e.g. not substituted) by one or two (e.g. one) groups independently selected from F, Cl, methyl, difluoromethyl, trifluoromethyl, methoxy, difluoromethoxy and trifluoromethoxy;
   R^{b} represents C₁₋₂alkyl optionally substituted by one or more F (e.g. methyl, ethyl, difluoromethyl or trifluoromethyl);
   each R^{c} and R^{q} independently represents C₁₋₆alkyl optionally substituted by one or more F;
   each R^{l}, R^{m}, Rⁿ and R^{o} independently represents H or C₁₋₆alkyl optionally substituted by one or more F; or
   R^{o} and R^{P} are linked together to form, along with the nitrogen atom to which they are attached, a 3- to 6-membered ring, which ring optionally contains one further heteroatom and which ring optionally is substituted by one or more F, one or more C₁₋₃alkyl each optionally and independently substituted by one or more F, or ═O;
   each R^{a1} independently represents H or C₁₋₃alkyl optionally substituted by one or more F; or
   two R^{a1} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O.

A further embodiment refers to compounds of formula I according to the second aspect of the invention, wherein A represents —CH₂-A¹;

A¹ represents phenyl substituted in the meta- or para-position (e.g para-position) by —B(OH)₂, —B(OMe)₂, 4,4,5,5-tetramethyl-1,3,2-dioxaborolan-2-yl or 6-methyl-1,3,6,2-dioxazaborocane-4,8-dion-2-yl;
X¹ represents C(R²);
X² represents N;
R² represents methyl, isopropyl, cyclopropyl, trifluoromethyl, methoxymethyl, benzyl or phenyl (e.g. methyl, cyclopropyl or trifluoromethyl);
R⁴ represents H;
R⁵ and R⁶ independently represent H, or preferably, F, Cl, or methyl, or more preferably at least one of (or preferably both of) R⁵ and R⁶ represent F, Cl, or methyl; and
R⁷ represents —NO₂.

In an embodiments A¹ represents phenyl substituted in the para-position by —B(OH)₂.

In another embodiments A¹ represents phenyl substituted in the para-position by 4,4,5,5-tetramethyl-1,3,2-dioxaborolan-2-yl.

In a further embodiments A¹ represents phenyl substituted in the para-position by 6-methyl-1,3,6,2-dioxazaborocane-4,8-dion-2-yl.

One embodiment refers to a compound selected from the group comprising
(4-((4-nitro-2-(trifluoromethyl)-1H-benzo[d]imidazol-1-yl)methyl)phenyl)boronic acid,
(4-((5,6-difluoro-2-methyl-4-nitro-1H-benzo[d]imidazol-1-yl)methyl)phenyl)boronic acid,
(4-((5,6-dichloro-2-methyl-4-nitro-1H-benzo[d]imidazol-1-yl)methyl)phenyl)boronic acid,
(4-((5,6-dichloro-2-cyclopropyl-4-nitro-1H-benzo[d]imidazol-1-yl)methyl)-phenyl)boronic acid,
(2-((5,6-dichloro-2-methyl-4-nitro-1H-benzo[d]imidazol-1-yl)methyl)phenyl)boronic acid,
(3-((5,6-dichloro-2-methyl-4-nitro-1H-benzo[d]imidazol-1-yl)methyl)phenyl)boronic acid,
5,6-dichloro-2-methyl-4-nitro-1-(4-(trifluoro-l4-boranyl)benzyl)-1H-benzo[d]imidazole, or potassium salt thereof,
5,6-dichloro-2-methyl-4-nitro-1-(4-(4,4,5,5-tetramethyl-1,3,2-dioxaborolan-2-yl)benzyl)-1H-benzo[d]imidazole,
2-(4-((5,6-dichloro-2-methyl-4-nitro-1H-benzo[d]imidazol-1-yl)methyl)phenyl)-6-methyl-1,3,6,2-dioxazaborocane-4,8-dione,
(4-(2-(5,6-dichloro-2-methyl-4-nitro-1H-benzo[d]imidazol-1-yl)ethyl)phenyl)boronic acid,
(4-((2,5,6-trimethyl-4-nitro-1H-benzo[d]imidazol-1-yl)methyl)phenyl)boronic acid, and
(4-((5,6-dimethyl-4-nitro-2-(trifluoromethyl)-1H-benzo[d]imidazol-1-yl)methyl)-phenyl)boronic acid, or
   a pharmaceutically acceptable salt thereof.

Another embodiment refers to compounds selected from the group comprising
(4-((5,6-dichloro-2-methyl-4-nitro-1H-benzo[d]imidazol-1-yl)methyl)phenyl)boronic acid,
(4-((5,6-dichloro-2-cyclopropyl-4-nitro-1H-benzo[d]imidazol-1-yl)methyl)-phenyl)boronic acid,
(2-((5,6-dichloro-2-methyl-4-nitro-1H-benzo[d]imidazol-1-yl)methyl)phenyl)boronic acid,
(3-((5,6-dichloro-2-methyl-4-nitro-1H-benzo[d]imidazol-1-yl)methyl)phenyl)boronic acid,
5,6-dichloro-2-methyl-4-nitro-1-(4-(trifluoro-l4-boranyl)benzyl)-1H-benzo[d]imidazole, or potassium salt thereof,
5,6-dichloro-2-methyl-4-nitro-1-(4-(4,4,5,5-tetramethyl-1,3,2-dioxaborolan-2-yl)benzyl)-1H-benzo[d]imidazole,
2-(4-((5,6-dichloro-2-methyl-4-nitro-1H-benzo[d]imidazol-1-yl)methyl)phenyl)-6-methyl-1,3,6,2-dioxazaborocane-4,8-dione,
(4-(2-(5,6-dichloro-2-methyl-4-nitro-1H-benzo[d]imidazol-1-yl)ethyl)phenyl)boronic acid,
(4-((2,5,6-trimethyl-4-nitro-1H-benzo[d]imidazol-1-yl)methyl)phenyl)boronic acid, and
(4-((5,6-dimethyl-4-nitro-2-(trifluoromethyl)-1H-benzo[d]imidazol-1-yl)methyl)phenyl)boronic acid,
   or a pharmaceutically acceptable salt thereof.

Another embodiment refers to a compound selected from the group comprising
2-(4-((5,6-dichloro-2-methyl-4-nitro-1H-benzo[d]imidazol-1-yl)methyl)phenyl)-6-methyl-1,3,6,2-dioxazaborocane-4,8-dione,
(4-((5,6-dichloro-2-methyl-4-nitro-1H-benzo[d]imidazol-1-yl)methyl)phenyl)boronic acid,
(4-((5,6-dichloro-2-cyclopropyl-4-nitro-1H-benzo[d]imidazol-1-yl)methyl)phenyl)boronic acid,
5,6-dichloro-2-methyl-4-nitro-1-(4-(4,4,5,5-tetramethyl-1,3,2-dioxaborolan-2-yl)benzyl)-1H-benzo[d]imidazole, and
5,6-dichloro-2-methyl-4-nitro-1-(4-(trifluoro-l4-boranyl)benzyl)-1H-benzo[d]imidazole, or potassium salt thereof,
   or a pharmaceutically acceptable salt thereof.

Unless indicated otherwise, all technical and scientific terms used herein have the same meaning as commonly understood by one of ordinary skill in the art to which this invention pertains.

Pharmaceutically-acceptable salts for any compound or scope of compounds as defined herein, include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Particular acid addition salts may include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxy-maleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxyethanesulphonate, 1- or 2-naphthalene-sulphonate or 1,5-naphthalenedisulphonate salts) or sulphate, pyrosulphate, bisulphate, sulphite, bisulphite, phosphate, monohydrogen-phosphate, dihydrogenphosphate, metaphosphate, pyrophosphate or nitrate salts, and the like.

Particular base addition salts may include salts formed with alkali metals (such as Na and K salts), alkaline earth metals (such as Mg and Ca salts), organic bases (such as ethanolamine, diethanolamine, triethanolamine, tromethamine and lysine) and inorganic bases (such as ammonia and aluminium hydroxide). Other base addition salts include Mg, Ca and. Further base salts may be K and Na salts. In one embodiment, the salt is a potassium salt.

For the avoidance of doubt, compounds of the invention may exist as solids, and the scope of the invention includes all amorphous, crystalline and part crystalline and hydrate forms thereof. Where compounds of the invention exist in crystalline and part crystalline forms, such forms may include solvates, which are included in the scope of the invention. Compounds of the invention may also exist in solution. The compounds of the invention may exist as oils.

Compounds of the invention may contain double bonds and may thus exist as E (entgegen) and Z (zusammen) geometric isomers about each individual double bond. All such isomers and mixtures of any of the compounds of the invention are included within the scope of the invention.

Compounds of the invention may also exhibit tautomerism. All tautomeric forms and mixtures thereof of any of the compounds of the invention are included within the scope of the invention.

Compounds of the invention may also contain one or more asymmetric carbon atoms and may therefore exhibit optical isomerism and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers, and mixtures thereof of any of the compounds of the invention are included within the scope of the invention.

As used herein, references to halo and/or halogen will independently refer to fluoro (F), chloro (Cl), bromo (Br) and iodo (I), for example, F and/or CI.

Unless otherwise specified, C_{1-q}alkyl groups (where q is the upper limit of the range, e.g. 2, 3, 4, 5, 6, or 2 to 12) defined herein may be straight-chain or, when there is a sufficient number (i.e. a minimum of two or three, as appropriate) of carbon atoms, be branched-chain, and/or cyclic (so forming a C_{3-q}cycloalkyl group). When there is a sufficient number (i.e. a minimum of four) of carbon atoms, such groups may also be part cyclic. Such alkyl groups may also be saturated or, when there is a sufficient number (i.e. a minimum of two) of carbon atoms, be unsaturated (forming, for example, a C_{2-q}alkenyl or a C_{2-q}alkynyl group).

Unless otherwise specified, C_{1-q}alkylene groups (where q is the upper limit of the range, e.g. 2, 3, 4, 5, 6, or 2 to 12) defined herein may (in a similar manner to the definition of C_{1-q}alkyl) be straight-chain or, when there is a sufficient number (i.e. a minimum of two or three, as appropriate) of carbon atoms, be branched-chain, and/or cyclic (so forming a C_{3-q}-cycloalkylene group, such as cyclopropylene). When there is a sufficient number (i.e. a minimum of four) of carbon atoms, such groups may also be part cyclic. Such alkylene groups may also be saturated or, when there is a sufficient number (i.e. a minimum of two) of carbon atoms, be unsaturated, i.e. containing one or more double and/or triple bonds (e.g. one or two double bonds, or one triple bond), forming, for example, a C_{2-q}alkenylene or a C_{2-q}alkynylene group. Particular alkylene groups that may be mentioned include those that are straight-chained or cyclic and saturated.

Heterocycloalkyl groups that may be mentioned include non-aromatic monocyclic and bicyclic heterocycloalkyl groups (which groups may further be bridged) in which at least one (e.g. one to four) of the atoms in the ring system is other than carbon (i.e. a heteroatom), and in which the total number of atoms in the ring system is between three and twelve (e.g. between five and ten and, most preferably, between three and eight, e.g. a 5- or 6-membered heterocycloalkyl group). Further, such heterocycloalkyl groups may be saturated or unsaturated containing one or more double and/or triple bonds, e.g. one or two double bonds, forming for example a C_{2-q} (e.g. C_{4-q}) heterocycloalkenyl (where q is the upper limit of the range) or a C_{7-q} heterocycloalkynyl group. C_{2-q} heterocycloalkyl groups that may be mentioned include 7-azabicyclo-[2.2.1]heptanyl, 6-azabicyclo[3.1.1]-heptanyl, 6-azabicyclo[3.2.1]-octanyl, 8-azabicyclo[3.2.1]octanyl, aziridinyl, azetidinyl, dihydropyranyl, dihydropyridyl, dihydropyrrolyl (including 2,5-dihydropyrrolyl), 1,3,2-dioxaborinane, 1,3,6,2-dioxazaborocane, 1,3,2□ dioxaborolane, dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo-[2.2.1]heptanyl, 6-oxabicyclo[3.2.1]-octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuryl, tetrahydropyridyl (such as 1,2,3,4-tetrahydropyridyl and 1,2,3,6-tetrahydropyridyl), thietanyl, thiiranyl, thiolanyl, tetrahydrothiopyranyl, thiomorpholinyl, trithianyl (including 1,3,5-trithianyl), tropanyl, and the like. Substituents on heterocycloalkyl groups may, where appropriate, be located on any atom in the ring system including a heteroatom. Further, in the case where the substituent is another cyclic compound, then the cyclic compound may be attached through a single atom on the heterocycloalkyl group, forming a so-called “spiro”-compound. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form. Examples of heterocycloalkyl groups are 3- to 8-membered heterocycloalkyl groups (e.g. 4- to 6-membered heterocycloalkyl groups).

The term “aryl”, when used herein, includes C₆₋₁₀ aromatic groups. Such groups may be monocyclic or bicyclic and, when bicyclic, be either wholly or partly aromatic. C₆₋₁₀ aryl groups that may be mentioned include phenyl, naphthyl, 1,2,3,4-tetrahydronaphthyl, indanyl, and the like (e.g. phenyl, naphthyl and the like). For the avoidance of doubt, the point of attachment of substituents on aryl groups may be via any carbon atom of the ring system.

The term “heteroaryl” (or heteroaromatic), when used herein, includes 5- to 11-membered heteroaromatic groups containing one or more heteroatoms selected from oxygen, nitrogen and/or sulfur. Such heteroaryl group may comprise one, or two rings, of which at least one is aromatic. Substituents on heteroaryl/heteroaromatic groups may, where appropriate, be located on any atom in the ring system including a heteroatom. The point of attachment of heteroaryl/heteroaromatic groups may be via any atom in the ring system including (where appropriate) a heteroatom. Bicyclic heteroaryl/heteroaromatic groups may comprise a benzene ring fused to one or more further aromatic or non-aromatic heterocyclic rings, in which instances, the point of attachment of the polycyclic heteroaryl/heteroaromatic group may be via any ring including the benzene ring or the heteroaryl/heteroaromatic or heterocycloalkyl ring. Examples of heteroaryl/heteroaromatic groups that may be mentioned include pyridinyl, pyrrolyl, furanyl, thiophenyl, oxadiazolyl, thiadiazolyl, thiazolyl, oxazolyl, pyrazolyl, triazolyl, tetrazolyl, isoxazolyl, isothiazolyl, imidazolyl, imidazopyrimidinyl, imidazothiazolyl, thienothiophenyl, pyrimidinyl, furopyridinyl, indolyl, azaindolyl, pyrazinyl, indazolyl, pyrimidinyl, quinolinyl, isoquinolinyl, quinazolinyl, benzofuranyl, benzothiophenyl, benzoimidazolyl, benzoxazolyl, benzothiazolyl, benzotriazolyl and purinyl. The oxides of heteroaryl/heteroaromatic groups are also embraced within the scope of the invention (e.g. the N-oxide). As stated above, heteroaryl groups includes polycyclic (e.g. bicyclic) groups where all rings are aromatic, and partly aromatic groups where at least one ring is aromatic and at least one other ring is not aromatic. Hence, other heteroaryl groups that may be mentioned include e.g. benzo[1,3]dioxolyl, benzo[1,4]dioxinyl, dihydrobenzo[disothiazolyl, 1,2-dihydrobenzo[d][1,2,3]diazaborininyl, 3,4-dihydro-1H-benzo[c][1,2]oxaborininyl, 1,3-dihydrobenzo[c][1,2]oxaborolyl, 3,4-dihydrobenz[1,4]oxazinyl, dihydrobenzothiophenyl, indolinyl, 5H,6H,7H-pyrrolo[1,2-b]pyrimidinyl, 1,2,3,4-tetrahydroquinolinyl, thiochromanyl and the like.

For the avoidance of doubt, as used herein, references to heteroatoms will take their normal meaning as understood by one skilled in the art. Particular heteroatoms that may be mentioned include phosphorus, selenium, tellurium, silicon, oxygen, nitrogen and sulphur (e.g. boron, oxygen, nitrogen and sulphur).

For the avoidance of doubt, references to polycyclic (e.g. bicyclic) groups (e.g. when employed in the context of heterocycloalkyl groups) will refer to ring systems wherein more than two scissions would be required to convert such rings into a straight chain, with the minimum number of such scissions corresponding to the number of rings defined (e.g. the term bicyclic may indicate that a minimum of two scissions would be required to convert the rings into a straight chain). For the avoidance of doubt, the term bicyclic (e.g. when employed in the context of heterocycloalkyl groups) may refer to groups in which the second ring of a two-ring system is formed between two adjacent atoms of the first ring, and may also refer to groups in which two non-adjacent atoms are linked by either an alkylene or heteroalkylene chain (as appropriate), which later groups may be referred to as bridged.

For the avoidance of doubt, when an aryl or an heteroaryl group is substituted with a group via a double bond, such as ═O, it is understood that the aryl or heteroaryl group is partly aromatic, i.e. the aryl or heteroaryl group consists of at least two rings where at least one ring is not aromatic.

Compounds and salts described in this specification may be isotopically-labelled compounds (or “radio-labelled”). In that instance, one or more atoms are replaced by an atom having an atomic mass or mass number different from the atomic mass or mass number most abundantly found in nature (i.e., naturally occurring). Examples of suitable isotopes that may be incorporated include ²H (also written as “D” for deuterium), ³H (also written as “T” for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The isotope that is used will depend on the specific application of that isotopically-labelled derivative. For example, for in vitro receptor labelling and competition assays, compounds that incorporate ³H or ¹⁴C are often useful. Deuterium (²H) may be incorporated in molecules instead of hydrogen (¹H) to modify certain properties, e.g. to reduce metabolism. For radio-imaging applications ¹¹C or ¹⁸F are often useful. In some embodiments, the isotope is ²H. In some embodiments, the isotope is ³H. In some embodiments, the radionuclide is ¹⁴C. In some embodiments, the isotope is ¹¹C. In some embodiments, the isotope is ¹⁸F.

For the avoidance of doubt, in cases in which the identity of two or more substituents in a compound of the invention may be the same, the actual identities of the respective substituents are not in any way interdependent. For example, in the situation in which two Y¹ groups are present, those Y¹ groups may be the same or different. Similarly, where two Y¹ groups are present and each represent halo, the halo groups in question may be the same or different. Likewise, when more than one R^{b1} is present and each independently represents C₁₋₄ alkyl substituted by one or more D⁴ group, the identities of each D⁴ are in no way interdependent.

All individual features mentioned herein may be taken in isolation or in combination with any other feature.

The skilled person will appreciate that compounds of the invention that are the subject of this invention include those that are stable. That is, compounds of the invention include those that are sufficiently robust to survive isolation e.g. from a reaction mixture, to a useful degree of purity.

All embodiments of the invention and particular features mentioned herein may be taken in isolation or in combination with any other embodiments and/or particular features mentioned herein (hence describing more particular embodiments and particular features as disclosed herein) without departing from the disclosure of the invention.

### Medical Uses

A third aspect of the invention relates to a compound according to the second aspect of the invention, as hereinbefore defined, including any and all embodiments mentioned above, for use in therapy, e.g. for use as a medicament.

In an embodiment of the invention, there is provided the use of a compound according to the first or second aspect of the invention, as hereinbefore defined, in the manufacture of a medicament for use in therapy.

In a further embodiment of the invention, there is provided a method of treating proliferative disorders comprising administering to a patient in need thereof a therapeutically effective amount of a compound of the invention according to the first or second aspect of the invention, including any and all embodiments mentioned above, as hereinbefore defined.

One embodiment of the third aspect relates to a compound of the invention according to the second aspect of the invention, as hereinbefore defined, including any and all embodiments mentioned above, for use in the treatment of proliferative disorders. In another embodiment the proliferative disorder is cancer. In a further embodiment the proliferative disorder is inflammation.

Another embodiment of the third aspect relates to a compound of the invention according to the first aspect of the invention, as hereinbefore defined, including any and all embodiments mentioned above, for use in the treatment of proliferative disorders. In another embodiment the proliferative disorder is cancer. In a further embodiment the proliferative disorder is inflammation.

The term “therapy” and “treatment” as used herein include prevention, prophylaxis, therapeutic and therapeutic, and the like.

The term “disorder” as used herein includes disease, condition, and the like.

The skilled person will understand that references to the treatment of a particular condition (or, similarly, to treating that condition) take their normal meanings in the field of medicine. In particular, the terms may refer to achieving a reduction in the severity of one or more clinical symptom associated with the condition. For example, in the case of a cancer, the term may refer to achieving a reduction of the amount of cancerous cells present (e.g. in the case of a cancer forming a solid tumour, indicated by a reduction in tumour volume). In the case of an inflammation or an inflammatory disorder, the term may refer to achieving a reduction of among others an amount of white blood cells.

As used herein, references to patients will refer to a living subject being treated, including mammalian e.g. human patients.

Although compounds of the invention may possess pharmacological activity as such, certain pharmaceutically-acceptable (e.g. “protected”) derivatives of compounds of the invention may exist or be prepared, which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised) may therefore be described as “prodrugs” of compounds of the invention.

As used herein, references to prodrugs will include compounds that form a compound of the invention, in an experimentally-detectable amount, within a predetermined time, following enteral or parenteral administration (e.g. oral or parenteral administration). All prodrugs of the compounds of the invention are included within the scope of the invention.

Furthermore, certain compounds of the invention may possess no or minimal pharmacological activity as such, but may be administered parenterally or orally, and thereafter be metabolised in the body to form compounds of the invention that possess pharmacological activity as such. Such compounds (which also includes compounds that may possess some pharmacological activity, but which activity is appreciably lower than that of the “active” compounds of the invention to which they are metabolised), may also be described as “prodrugs”.

Thus, the compounds of the invention are believed to be useful because they possess pharmacological activity, and/or are metabolised in the body following oral or parenteral administration to form compounds, which possess pharmacological activity.

In one embodiment the compound of the invention according to the first or second aspect of the invention, including any and all embodiments mentioned above, as hereinbefore defined, is used for the treatment cancer, whereby the cancer is selected from the group comprising:

soft tissue cancers, such as sarcoma (e.g. angiosarcoma, fibrosarcoma, rhabdomyosarcoma, liposarcoma), myxoma, rhabdomyoma, fibroma, lipoma and teratoma;
lung cancers, such as bronchogenic carcinoma (e.g. squamous cell, undifferentiated small cell, undifferentiated large cell, adenocarcinoma), alveolar (or bronchiolar) carcinoma, bronchial adenoma, sarcoma, lymphoma, chondromatous hamartoma, mesothelioma;
gastrointestinal cancers: such as esophagus (e.g. squamous cell carcinoma, adenocarcinoma, leiomyosarcoma, lymphoma), stomach (e.g. carcinoma, lymphoma, leiomyosarcoma), pancreatic cancers (e.g. ductal adenocarcinoma, insulinoma, glucagonoma, gastrinoma, carcinoid tumors, vipoma), small bowel cancers (e.g. adenocarcinoma, lymphoma, carcinoid tumors, Kaposi's sarcoma, leiomyoma, hemangioma, lipoma, neurofibroma, fibroma), large bowel cancers (e.g. adenocarcinoma, tubular adenoma, villous adenoma, hamartoma, leiomyoma);
genitourinary tract cancers, such as cancers of the kidney (adenocarcinoma, Wilm's tumor (nephroblastoma), lymphoma, leukemia), bladder and urethra (e.g. squamous cell carcinoma, transitional cell carcinoma, adenocarcinoma), prostate (e.g. adenocarcinoma, sarcoma), testis (e.g. seminoma, teratoma, embryonal carcinoma, teratocarcinoma, choriocarcinoma, sarcoma, interstitial cell carcinoma, fibroma, fibroadenoma, adenomatoid tumors, lipoma);
liver cancers, such as hepatoma (e.g. hepatocellular carcinoma), cholangiocarcinoma, hepatoblastoma, angiosarcoma, hepatocellular adenoma, hemangioma;
bone cancers, such as osteogenic sarcoma (e.g. osteosarcoma), fibrosarcoma, malignant fibrous histiocytoma, chondrosarcoma, Ewing's sarcoma, malignant lymphoma (e.g. reticulum cell sarcoma), multiple myeloma, malignant giant cell tumor chordoma, osteochronfroma (e.g. osteocartilaginous exostoses), benign chondroma, chondroblastoma, chondromyxofibroma, osteoid osteoma and giant cell tumors;
cancers of the head and/or nervous system, such as cancer of the skull (e.g. osteoma, hemangioma, granuloma, xanthoma, osteitis deformans), meninges (e.g. meningioma, meningiosarcoma, gliomatosis), brain (e.g. astrocytoma, medulloblastoma, glioma, ependymoma, germinoma (pinealoma), glioblastoma multiform, oligodendroglioma, schwannoma, retinoblastoma, congenital tumors), spinal cord (e.g. neurofibroma, meningioma, glioma, sarcoma);
gynecological cancers, such as cancers of the uterus (e.g. endometrial carcinoma), cervix (e.g. cervical carcinoma, pre-tumor cervical dysplasia), ovaries (e.g. ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), cancers of the vulva (e.g. squamous cell carcinoma, intraepithelial carcinoma, adenocarcinoma, fibrosarcoma, melanoma), vagina (e.g. clear cell carcinoma, squamous cell carcinoma, botryoid sarcoma (embryonal rhabdomyosarcoma), fallopian tubes (e.g. carcinoma);
hematologic cancers, such as cancers of the blood and bone marrow (e.g. myeloid leukemia (acute and chronic), acute lymphoblastic leukemia, chronic lymphocytic leukemia, myeloproliferative disorders, multiple myeloma, myelodysplastic syndrome), Hodgkin's disorder, non-Hodgkin's lymphoma (malignant lymphoma);
skin cancers, such as malignant melanoma, basal cell carcinoma, squamous cell carcinoma, Kaposi's sarcoma, moles dysplastic nevi, lipoma, angioma, dermatofibroma, keloids;
adrenal glands cancers; and
neuroblastomas.

As used herein, references to cancerous cells and the like will include references to a cell afflicted by any one of the above identified conditions.

In one embodiment the cancer may be selected from the group comprising acute myeloid leukaemia, acute lymphocytic leukaemia, myelodysplastic syndrome, chronic myelomonocytic leukaemia, lymphomas, advanced stomach cancer, oesophageal cancer and ovarian cancer.

In another embodiment the cancer is selected from the group comprising acute myeloid leukaemia, acute lymphocytic leukaemia, myelodysplastic syndrome, chronic myelomonocytic leukaemia and lymphomas.

In another embodiment the cancer is selected from the group comprising acute myeloid leukaemia and myelodysplastic syndrome.

The skilled person will understand that treatment with compounds of the invention may comprise (i.e. be combined with) further treatment(s) for the same condition. In particular, treatment with compounds of the invention may be combined with other means for the treatment of a proliferative disorder, e.g. cancer, and/or inflammation, such as treatment with one or more other therapeutic agent that is useful in the treatment of cancer and/or one or more physical method used in the treatment of cancer (such as treatment through surgery), as known to those skilled in the art.

Thus, there is also provided a method of treating a proliferative disorder, e.g. cancer and/or inflammation, in a patient in need thereof wherein the patient is administered a therapeutically effective amount of compound of the invention according to the second aspect of the invention, as hereinbefore defined, including any and all embodiments mentioned above, in combination with treatment by radiotherapy, simultaneously, concomitantly or sequentially.

The term “inflammation” will be understood by those skilled in the art to include any condition characterised by a localised or a systemic protective response, which may be elicited by physical trauma, infection, chronic disorders, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disorder, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

In one embodiment the compound of the invention according to the first or second aspect of the invention, including any and all embodiments mentioned above, as hereinbefore defined, is used for the treatment of inflammation selected from the group comprising allergic disorders, asthma, childhood wheezing, chronic obstructive pulmonary disorder, bronchopulmonary dysplasia, cystic fibrosis, interstitial lung disorder (e.g. sarcoidosis, pulmonary fibrosis, scleroderma lung disorder, and usual interstitial in pneumonia), ear nose and throat disorders (e.g. rhinitis, nasal polyposis, and otitis media), eye disorders (e.g. conjunctivitis and giant papillary conjunctivitis), skin disorders (e.g. psoriasis, dermatitis, and eczema), rheumatic disorders (e.g. rheumatoid arthritis, arthrosis, psoriasis arthritis, osteoarthritis, systemic lupus erythematosus, systemic sclerosis), vasculitis (e.g. Henoch-Schonlein purpura, Löffler's syndrome and Kawasaki disorder), cardiovascular disorders (e.g. atherosclerosis), gastrointestinal disorders (e.g. eosinophilic disorders in the gastrointestinal system, inflammatory bowel disorder, irritable bowel syndrome, colitis, celiaci and gastric haemorrhagia), urologic disorders (e.g. glomerulonephritis, interstitial cystitis, nephritis, nephropathy, nephrotic syndrome, hepatorenal syndrome, and nephrotoxicity), disorders of the central nervous system (e.g. cerebral ischemia, spinal cord injury, migraine, multiple sclerosis, and sleep-disordered breathing), endocrine disorders (e.g. autoimmune thyreoiditis, diabetes-related inflammation), urticaria, anaphylaxis, angioedema, oedema in Kwashiorkor, dysmenorrhoea, burn-induced oxidative injury, multiple trauma, pain, toxic oil syndrome, endotoxin chock, sepsis, bacterial infections (e.g. from Helicobacter pylori, Pseudomonas aerugiosa or Shigella dysenteriae), fungal infections (e.g. vulvovaginal candidasis), viral infections (e.g. hepatitis, meningitis, parainfluenza and respiratory syncytial virus), sickle cell anemia and hypereosinofilic syndrome. In particular, compounds of the invention may be useful in treating allergic disorders, asthma, rhinitis, conjunctivitis, COPD, cystic fibrosis, dermatitis, urticaria, eosinophilic gastrointestinal disorders, inflammatory bowel disorder, rheumatoid arthritis, osteoarthritis and pain.

In one embodiment the compound of the invention according to the first or second aspect of the invention, including any and all embodiments mentioned above, as hereinbefore defined, is used for the treatment of proliferative disorders such as autoimmune disorders, allergic disorders and hyperinflammatory disorders. These disorders or disorders are conditions where a mammal's immune system starts reacting against its own tissues. In one embodiment, the autoimmune disorder may be selected from rheumatoid arthritis, multiple sclerosis, inflammatory bowel disorder including Crohn's disorder and ulcerative colitis, systemic lupus erythematosus, autoimmune uveitis, type I diabetes, dermatomyesitis, Goodpasteure's syndrome, Graves' disorder, Guillian-Barré Syndrome (GBS), Hashimotos Disorder, Mixed connective tissue disorder, Myasthenia gravis, Pemphigus vulgaris, Pernicious anemia, Psoriasis, Polymyositis, Primary biliary cirrhosis, Sjögren's syndrome, Giant cell arteritis, ulcerative colitis, vasculitis, Wegener's granulomatosis, Churg-Strauss syndrome and iopathic thrombocytopenic purpura. Most preferably the autoimmune disorder is selected from rheumatoid arthritis and multiple sclerosis. In another embodiment, the inflammatory (e.g. chronic inflammatory) disorder is selected from celiac disorder, vasculitis, lupus, chronic obstructive pulmonary disorder (COPD), irritable bowel disorder, atherosclerosis, arthritis and psoriasis.

In another embodiment, the inflammatory disorder is selected from the group comprising Asthma, Allergic disorders, Atopic dermatitis (eczema), Crohn's disease, Hay fever, Idiopathic hypereosinophilic syndrome, Ulcerative colitis, Churg-Strauss syndrome, Löffler syndrome, Drug allergy, Lupus and Hypereosinophilic Syndrome.

Compounds of the invention may be administered orally, intravenously, subcutaneously, buccally, rectally, dermally, nasally, tracheally, bronchially, by any other parenteral route or via inhalation, in a pharmaceutically acceptable dosage form. Alternatively, particularly where compounds of the invention are intended to act locally, compounds of the invention may be administered topically. The skilled person will understand that compounds of the invention may act systemically and/or locally (i.e. at a particular site).

As used herein, the term effective amount refers to an amount of a compound that confers a therapeutic effect on the treated patient. The effect may be objective (i.e. measurable by some test or marker) or subjective (i.e. the subject gives an indication of or feels an effect). Compounds of the invention may be administered at varying doses. Oral, pulmonary and topical dosages (and subcutaneous dosages, although these dosages may be relatively lower) may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 2000 mg, for example between about 0.1 mg to about 500 mg, or between 1 mg to about 100 mg, of the active ingredient. Intravenously, the most preferred doses will range from about 0.001 to about 10 mg/kg of body weight per hour (mg/kg/hour) during constant rate infusion. Advantageously, compounds may be administered in a single daily dose, or the total daily dosage may be administered in divided doses of two, three or four times daily. The above-mentioned dosages are exemplary of the average case; there can, of course, be individual instances where higher or lower dosage ranges are merited.

In any event, the physician, or the skilled person, will be able to determine the actual dosage which will be most suitable for an individual patient, which will vary depending on the route of administration, the type and severity of the condition that is to be treated, as well as the species, age, weight, sex, renal function, hepatic function and response of the particular patient to be treated.

### Pharmaceutical Formulation

According to a fourth aspect of the invention there is provided a pharmaceutical formulation including a compound of the invention according to the first or second aspect of the invention, including any and all embodiments mentioned above, as hereinbefore defined, including any and all embodiments mentioned above, in admixture with one or more pharmaceutically acceptable adjuvant, diluent and/or carrier.

For the purpose of the present invention, the term formulation is used synonymously with the term “composition”, unless otherwise specified or apparent from the context

Compounds of the invention may be administered in the form of tablets or capsules, e.g. time-release capsules that are taken orally. Alternatively, the compounds of the invention may be in a liquid form and may be taken orally or by injection. In particular, injection may take place using conventional means, and may include the use of microneedles. The compounds of the invention may also be in the form of suppositories, or, creams, gels, and foams e.g. that can be applied to the skin. In addition, they may be in the form of an inhalant that is applied nasally or via the lungs.

Depending on e.g. potency and physical characteristics of the compound of the invention (i.e. active ingredient), pharmaceutical formulations that may be mentioned include those in which the active ingredient is present in at least 1% (or at least 10%, at least 30% or at least 50%) by weight of the total weight of the formulation. That is, the ratio of active ingredient to the other components (i.e. the addition of adjuvant, diluent and carrier) of the pharmaceutical formulation is at least 1:99 (or at least 10:90, at least 30:70 or at least 50:50) by weight.

Pharmaceutical formulations, as described herein, may be prepared in accordance with standard and/or accepted pharmaceutical practice.

In one embodiment of the fourth aspect of the invention, there is provided a process for the preparation of a pharmaceutical formulation, as hereinbefore defined, which process comprises bringing into association compound of the invention according to the first or second aspect of the invention, including any and all embodiments mentioned above, as hereinbefore defined, or a pharmaceutically acceptable salt thereof, with one or more pharmaceutically-acceptable adjuvant, diluent and/or carrier.

The invention relates to a pharmaceutical formulation including compound of the invention according to the first or second aspect of the invention, including any and all embodiments mentioned above, as hereinbefore defined, in admixture with one or more pharmaceutically acceptable adjuvant, diluent and/or carrier, for use in therapy, such as treatment of a proliferative disorder, e.g. cancer and/or inflammation.

### Combination Products

As described herein, compounds of the invention may also be combined with one or more other therapeutic agents. Such combination products that provide for the administration of a compound of the invention in conjunction with one or more other therapeutic agent may be presented either as separate formulations, wherein at least one formulation comprises a compound of the invention, and at least one formulation comprises the one or more other therapeutic agent. A combination product may also be presented as a single formulation comprising a compound of the invention and the one or more other therapeutic agent.

According to one embodiment of a fifth aspect of the invention, there is provided a combination product comprising:
(A) a compound of the invention according to the first or second aspect of the invention, including any and all embodiments mentioned above, as hereinbefore defined, in admixture with one or more pharmaceutically-acceptable adjuvant, diluent and/or carrier and
(B) one or more other therapeutic agent in admixture with one or more pharmaceutically-acceptable adjuvant, diluent and/or carrier.

According to another embodiment of the fifth aspect of the invention, there is provided a combination product comprising:
(C) a compound of the invention according to the first or second aspect of the invention, including any and all embodiments mentioned above, as hereinbefore defined, and
(D) one or more other therapeutic agent,
   in admixture with one or more pharmaceutically-acceptable adjuvant, diluent and/or carrier,

In a sixth aspect of the invention there is provided a kit-of-parts comprising the combination product defined above.

In one embodiment of the sixth aspect there is provided a kit-of-parts comprising

(A) a compound of the invention according to the first or second aspect of the invention, including any and all embodiments mentioned above, as hereinbefore defined, in admixture with one or more pharmaceutically-acceptable adjuvant, diluent and/or carrier, and
(B) one or more other therapeutic agent in admixture with one or more a pharmaceutically-acceptable adjuvant, diluent and/or carrier,
suitable for simultaneous, concomitantly or sequentially administration.

Pharmaceutical formulations, combination products and kits-of-parts, as described herein, may be prepared in accordance with standard and/or accepted pharmaceutical practice.

In one embodiment, there is provided a process for the preparation of a combination product or kit-of-parts as hereinbefore defined, which process comprises bringing into association a compound of the invention according to the first or second aspect of the invention, including any and all embodiments mentioned above, as hereinbefore defined, with the one or more other therapeutic agent and one or more pharmaceutically-acceptable adjuvant, diluent and/or carrier.

The invention relates to a combination products and kits-of-parts including a compound of the invention according to the first or second aspect of the invention, including any and all embodiments mentioned above, as hereinbefore defined, including any and all embodiments mentioned above, for use in therapy, such as treatment of a proliferative disorder, e.g. cancer and/or inflammation.

As used herein, references to bringing into association will mean that the two components are rendered suitable for administration in conjunction with each other, e.g. the compounds or agents or pharmaceutically acceptable salts thereof are mixed together with one or more pharmaceutically-acceptable adjuvant, diluent and/or carrier.

In relation to the process for the preparation of a kit of parts as hereinbefore defined, by bringing the two components “into association with” each other, the compounds or agents comprised in the kit of parts may be:

(i) provided as separate pharmaceutical formulations,
(ii) packaged and presented together in a “combination pack” for use in conjunction with each other in a combination therapy.

Examples of therapeutic agents (component (B)) that may be useful in combination with compounds of this invention are selected from the group comprising of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors; kinase inhibitors; angiogenesis inhibitors; immunotherapeutic agents; pro-apoptotic agents; and cell cycle signaling inhibitors. Additional combination therapy comprises radiation therapy. Further therapeutic agents that are useful in the treatment of a respiratory disorder (e.g. leukotriene receptor antagonists (LTRas), glucocorticoids, antihistamines, beta-adrenergic drugs, anticholinergic drugs and PDE₄ inhibitors and/or other therapeutic agents that are useful in the treatment of a respiratory disorder) and/or other therapeutic agents that are useful in the treatment of inflammation and disorders with an inflammatory component (e.g. NSAIDs, coxibs, corticosteroids, analgesics, inhibitors of 5-lipoxygenase, inhibitors of FLAP (5-lipoxygenase activting protein), immunosuppressants and sulphasalazine and related compounds and/or other therapeutic agents that are useful in the treatment of inflammation).

In one embodiment of the present invention, the one or more combination agent is a nucleoside analogue such as a cytidine analogue. In another embodiment, the one or more cytidine analogue is selected from the group comprising cytarabine, fludarabine, cladribine, clofarabine, nelarabine, capecitabine, floxuridine, deoxycoformycin, azacitidine (also known as 5-azacytidine), decitabine, gemcitabine, sapacitabine, zebularine, fluorouracil and 4′-thio-2′-deoxycytidine. In another embodiment, the cytidine analogue is selected from the group comprising azacitidine, decitabine and gemcitabine.

In one embodiment, the combination products as defined above, comprises a compound of the invention selected from the group comprising
1-(4-methoxybenzyl)-2-methyl-4-nitro-1H-benzo[d]imidazole,
1-(4-methoxybenzyl)-4-nitro-2-phenyl-1H-benzo[d]imidazole,
2-benzyl-1-(4-methoxybenzyl)-4-nitro-1H-benzo[d]imidazole,
1-(4-methoxybenzyl)-4-nitro-2-(trifluoromethyl)-1H-benzo[d]imidazole,
2-methoxy-1-(4-methoxybenzyl)-4-nitro-1H-benzo[d]imidazole,
1-(4-methoxybenzyl)-2-(methoxymethyl)-4-nitro-1H-benzo[d]imidazole,
2-isopropyl-1-(4-methoxybenzyl)-4-nitro-1H-benzo[d]imidazole,
(4-((4-nitro-2-(trifluoromethyl)-1H-benzo[d]imidazol-1-yl)methyl)phenyl)boronic acid,
5,6-difluoro-1-(4-methoxybenzyl)-2-methyl-4-nitro-1H-benzo[d]imidazole,
1-benzyl-5,6-difluoro-2-methyl-4-nitro-1H-benzo[d]imidazole,
4-((5,6-difluoro-2-methyl-4-nitro-1H-benzo[d]imidazol-1-yl)methyl)benzonitrile,
5,6-difluoro-1-(4-fluorobenzyl)-2-methyl-4-nitro-1H-benzo[d]imidazole,
1-(4-(1H-1,2,4-triazol-1-yl)benzyl)-5,6-difluoro-2-methyl-4-nitro-1H-benzo[d]imidazole,
5,6-difluoro-2-methyl-4-nitro-1-(1-phenylethyl)-1H-benzo[d]imidazole,
(4-((5,6-difluoro-2-methyl-4-nitro-1H-benzo[d]imidazol-1-yl)methyl)phenyl)boronic acid,
5,6-dichloro-2-methyl-4-nitro-1-phenyl-1H-benzo[d]imidazole,
5,6-dichloro-2-methyl-4-nitro-1-(phenylsulfonyl)-1H-benzo[d]imidazole,
(5,6-dichloro-2-methyl-4-nitro-1H-benzo[d]imidazol-1-yl)(phenyl)methanone,
1-benzyl-5,6-dichloro-2-methyl-4-nitro-1H-benzo[d]imidazole,
5,6-dichloro-1-(4-methoxybenzyl)-2-methyl-4-nitro-1H-benzo[d]imidazole,
5,6-dichloro-1-(3-methoxybenzyl)-2-methyl-4-nitro-1H-benzo[d]imidazole,
4-((5,6-dichloro-2-methyl-4-nitro-1H-benzo[d]imidazol-1-yl)methyl)phenyl acetate,
N-(4-((5,6-dichloro-2-methyl-4-nitro-1H-benzo[d]imidazol-1-yl)methyl)phenyl)-acetamide,
4-((5,6-dichloro-2-methyl-4-nitro-1H-benzo[d]imidazol-1-yl)methyl)aniline,
4-((5,6-dichloro-2-methyl-4-nitro-1H-benzo[d]imidazol-1-yl)methyl)phenol,
5,6-dichloro-2-methyl-1-(4-methylbenzyl)-4-nitro-1H-benzo[d]imidazole,
4-((5,6-dichloro-2-methyl-4-nitro-1H-benzo[d]imidazol-1-yl)methyl)benzaldehyde,
4-((5,6-dichloro-2-methyl-4-nitro-1H-benzo[d]imidazol-1-yl)methyl)benzaldehyde oxime,
1-(4-((5,6-dichloro-2-methyl-4-nitro-1H-benzo[d]imidazol-1-yl)methyl)phenyl)prop-2-en-1-ol,
1-(4-((5,6-dichloro-2-methyl-4-nitro-1H-benzo[d]imidazol-1-yl)methyl)phenyl)prop-2-en-1-one,
2-bromo-5-((5,6-dichloro-2-methyl-4-nitro-1H-benzo[d]imidazol-1-yl)methyl)benzaldehyde,
N-(4-((5,6-dichloro-2-methyl-4-nitro-1H-benzo[d]imidazol-1-yl)methyl)phenyl)acrylamide,
5,6-dichloro-1-(2-(3,5-dimethyl-1H-pyrazol-4-yl)ethyl)-2-methyl-4-nitro-1H-benzo[d]imidazole,
1-(4-(1H-pyrazol-1-yl)benzyl)-5,6-dichloro-2-methyl-4-nitro-1H-benzo[d]imidazole,
1-(4-(1H-1,2,4-triazol-1-yl)benzyl)-5,6-dichloro-2-methyl-4-nitro-1H-benzo[d]imidazole,
1-(4-(1H-pyrrol-1-yl)benzyl)-5,6-dichloro-2-methyl-4-nitro-1H-benzo[d]imidazole,
4-((5,6-dichloro-2-methyl-4-nitro-1H-benzo[d]imidazol-1-yl)methyl)benzoic acid,
methyl 4-((5,6-dichloro-2-methyl-4-nitro-1H-benzo[d]imidazol-1-yl)methyl)-benzoate,
4-((5,6-dichloro-2-methyl-4-nitro-1H-benzo[d]imidazol-1-yl)methyl)benzamide,
4-((5,6-dichloro-2-methyl-4-nitro-1H-benzo[d]imidazol-1-yl)methyl)benzonitrile,
5,6-dichloro-2-methyl-1-(4-(methylsulfonyl)benzyl)-4-nitro-1H-benzo[d]imidazole,
4-((5,6-dichloro-2-methyl-4-nitro-1H-benzo[d]imidazol-1-yl)methyl)-2-methylthiazole,
5,6-dichloro-1-((6-chloropyridin-3-yl)methyl)-2-methyl-4-nitro-1H-benzo[d]-imidazole,
(4-((5,6-dichloro-2-methyl-4-nitro-1H-benzo[d]imidazol-1-yl)methyl)phenyl)boronic acid,
(4-((5,6-dichloro-2-cyclopropyl-4-nitro-1H-benzo[d]imidazol-1-yl)methyl)phenyl)boronic acid
(2-((5,6-dichloro-2-methyl-4-nitro-1H-benzo[d]imidazol-1-yl)methyl)phenyl)boronic acid,
(3-((5,6-dichloro-2-methyl-4-nitro-1H-benzo[d]imidazol-1-yl)methyl)phenyl)boronic acid,
5,6-dichloro-2-methyl-4-nitro-1-(4-(trifluoro-l4-boranyl)benzyl)-1H-benzo[d]imidazole, or potassium salt thereof,
5,6-dichloro-2-methyl-4-nitro-1-(4-(4,4,5,5-tetramethyl-1,3,2-dioxaborolan-2-yl)benzyl)-1H-benzo[d]imidazole,
2-(4-((5,6-dichloro-2-methyl-4-nitro-1H-benzo[d]imidazol-1-yl)methyl)phenyl)-6-methyl-1,3,6,2-dioxazaborocane-4,8-dione,
(4-(2-(5,6-dichloro-2-methyl-4-nitro-1H-benzo[d]imidazol-1-yl)ethyl)phenyl)boronic acid,
2,5,6-trimethyl-4-nitro-1-(3,4,5-trimethoxybenzyl)-1H-benzo[d]imidazole,
1-((6-chlorobenzo[d][1,3]dioxol-5-yl)methyl)-2,5,6-trimethyl-4-nitro-1H-benzo[d]imidazole,
1-(4-methoxybenzyl)-2,5,6-trimethyl-4-nitro-1H-benzo[d]imidazole,
1-(4-chlorobenzyl)-2,5,6-trimethyl-4-nitro-1H-benzo[d]imidazole,
1-(4-fluorobenzyl)-2,5,6-trimethyl-4-nitro-1H-benzo[d]imidazole,
2,5,6-trimethyl-4-nitro-1-(4-(trifluoromethoxy)benzyl)-1H-benzo[d]imidazole,
1-(4-methoxybenzyl)-5,6-dimethyl-4-nitro-2-(trifluoromethyl)-1H-benzo[d]-imidazole,
2,5,6-trimethyl-4-nitro-1-(1-phenylethyl)-1H-benzo[d]imidazole,
(4-((2,5,6-trimethyl-4-nitro-1H-benzo[d]imidazol-1-yl)methyl)phenyl)boronic acid,
(4-((5,6-dimethyl-4-nitro-2-(trifluoromethyl)-1H-benzo[d]imidazol-1-yl)methyl)-phenyl)boronic acid,
1-(4-methoxybenzyl)-4-nitro-1H-benzo[d]imidazole,
(4-((4-nitro-1H-benzo[d]imidazol-1-yl)methyl)phenyl)boronic acid,
(4-((5,5,7,7-tetramethyl-4-nitro-6,7-dihydroindeno[5,6-d]imidazol-1 (5H)-yl)methyl)phenyl)boronic acid,
2-benzyl-1-(4-methoxybenzyl)-7-nitro-1H-benzo[d]imidazole,
2-methoxy-1-(4-methoxybenzyl)-7-nitro-1H-benzo[d]imidazole,
1-(4-methoxybenzyl)-7-nitro-2-phenyl-1H-benzo[d]imidazole and
(4-((5,6-difluoro-2-methyl-7-nitro-1H-benzo[d]imidazol-1-yl)methyl)phenyl)boronic acid,
   or a pharmaceutically acceptable salt thereof,
   and
   a therapeutic agent selected from the group comprising of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors; kinase inhibitors; angiogenesis inhibitors; immunotherapeutic agents; pro-apoptotic agents; and cell cycle signaling inhibitors,
   leukotriene receptor antagonists (LTRas), glucocorticoids, antihistamines, beta-adrenergic drugs, anticholinergic drugs and PDE₄ inhibitors, NSAIDs, coxibs, corticosteroids, analgesics, inhibitors of 5-lipoxygenase, inhibitors of FLAP (5-lipoxygenase activting protein), immunosuppressants.

In another embodiment, the combination products as defined above, comprises a compound of the invention selected from the group comprising
1-(4-methoxybenzyl)-2-methyl-4-nitro-1H-benzo[d]imidazole,
1-(4-methoxybenzyl)-4-nitro-2-phenyl-1H-benzo[d]imidazole,
2-benzyl-1-(4-methoxybenzyl)-4-nitro-1H-benzo[d]imidazole,
1-(4-methoxybenzyl)-4-nitro-2-(trifluoromethyl)-1H-benzo[d]imidazole,
2-methoxy-1-(4-methoxybenzyl)-4-nitro-1H-benzo[d]imidazole,
1-(4-methoxybenzyl)-2-(methoxymethyl)-4-nitro-1H-benzo[d]imidazole,
2-isopropyl-1-(4-methoxybenzyl)-4-nitro-1H-benzo[d]imidazole,
(4-((4-nitro-2-(trifluoromethyl)-1H-benzo[d]imidazol-1-yl)methyl)phenyl)boronic acid,
5,6-difluoro-1-(4-methoxybenzyl)-2-methyl-4-nitro-1H-benzo[d]imidazole,
1-benzyl-5,6-difluoro-2-methyl-4-nitro-1H-benzo[d]imidazole,
4-((5,6-difluoro-2-methyl-4-nitro-1H-benzo[d]imidazol-1-yl)methyl)benzonitrile,
5,6-difluoro-1-(4-fluorobenzyl)-2-methyl-4-nitro-1H-benzo[d]imidazole,
1-(4-(1H-1,2,4-triazol-1-yl)benzyl)-5,6-difluoro-2-methyl-4-nitro-1H-benzo[d]imidazole,
5,6-difluoro-2-methyl-4-nitro-1-(1-phenylethyl)-1H-benzo[d]imidazole,
(4-((5,6-difluoro-2-methyl-4-nitro-1H-benzo[d]imidazol-1-yl)methyl)phenyl)boronic acid,
5,6-dichloro-2-methyl-4-nitro-1-phenyl-1H-benzo[d]imidazole,
5,6-dichloro-2-methyl-4-nitro-1-(phenylsulfonyl)-1H-benzo[d]imidazole,
(5,6-dichloro-2-methyl-4-nitro-1H-benzo[d]imidazol-1-yl)(phenyl)methanone,
1-benzyl-5,6-dichloro-2-methyl-4-nitro-1H-benzo[d]imidazole,
5,6-dichloro-1-(4-methoxybenzyl)-2-methyl-4-nitro-1H-benzo[d]imidazole,
5,6-dichloro-1-(3-methoxybenzyl)-2-methyl-4-nitro-1H-benzo[d]imidazole,
4-((5,6-dichloro-2-methyl-4-nitro-1H-benzo[d]imidazol-1-yl)methyl)phenyl acetate,
N-(4-((5,6-dichloro-2-methyl-4-nitro-1H-benzo[d]imidazol-1-yl)methyl)phenyl)-acetamide,
4-((5,6-dichloro-2-methyl-4-nitro-1H-benzo[d]imidazol-1-yl)methyl)aniline,
4-((5,6-dichloro-2-methyl-4-nitro-1H-benzo[d]imidazol-1-yl)methyl)phenol,
5,6-dichloro-2-methyl-1-(4-methylbenzyl)-4-nitro-1H-benzo[d]imidazole,
4-((5,6-dichloro-2-methyl-4-nitro-1H-benzo[d]imidazol-1-yl)methyl)benzaldehyde,
4-((5,6-dichloro-2-methyl-4-nitro-1H-benzo[d]imidazol-1-yl)methyl)benzaldehyde oxime,
1-(4-((5,6-dichloro-2-methyl-4-nitro-1H-benzo[d]imidazol-1-yl)methyl)phenyl)prop-2-en-1-ol,
1-(4-((5,6-dichloro-2-methyl-4-nitro-1H-benzo[d]imidazol-1-yl)methyl)phenyl)prop-2-en-1-one,
2-bromo-5-((5,6-dichloro-2-methyl-4-nitro-1H-benzo[d]imidazol-1-yl)methyl)-benzaldehyde,
N-(4-((5,6-dichloro-2-methyl-4-nitro-1H-benzo[d]imidazol-1-yl)methyl)phenyl)-acrylamide,
5,6-dichloro-1-(2-(3,5-dimethyl-1H-pyrazol-4-yl)ethyl)-2-methyl-4-nitro-1H-benzo[d]imidazole,
1-(4-(1H-pyrazol-1-yl)benzyl)-5,6-dichloro-2-methyl-4-nitro-1H-benzo[d]imidazole,
1-(4-(1H-1,2,4-triazol-1-yl)benzyl)-5,6-dichloro-2-methyl-4-nitro-1H-benzo[d]imidazole,
1-(4-(1H-pyrrol-1-yl)benzyl)-5,6-dichloro-2-methyl-4-nitro-1H-benzo[d]imidazole,
4-((5,6-dichloro-2-methyl-4-nitro-1H-benzo[d]imidazol-1-yl)methyl)benzoic acid,
methyl 4-((5,6-dichloro-2-methyl-4-nitro-1H-benzo[d]imidazol-1-yl)methyl)-benzoate,
4-((5,6-dichloro-2-methyl-4-nitro-1H-benzo[d]imidazol-1-yl)methyl)benzamide,
4-((5,6-dichloro-2-methyl-4-nitro-1H-benzo[d]imidazol-1-yl)methyl)benzonitrile,
5,6-dichloro-2-methyl-1-(4-(methylsulfonyl)benzyl)-4-nitro-1H-benzo[d]imidazole,
4-((5,6-dichloro-2-methyl-4-nitro-1H-benzo[d]imidazol-1-yl)methyl)-2-methylthiazole,
5,6-dichloro-1-((6-chloropyridin-3-yl)methyl)-2-methyl-4-nitro-1H-benzo[d]imidazole,
(4-((5,6-dichloro-2-methyl-4-nitro-1H-benzo[d]imidazol-1-yl)methyl)phenyl)boronic acid,
(4-((5,6-dichloro-2-cyclopropyl-4-nitro-1H-benzo[d]imidazol-1-yl)methyl)-phenyl)boronic acid,
(2-((5,6-dichloro-2-methyl-4-nitro-1H-benzo[d]imidazol-1-yl)methyl)phenyl)boronic acid,
(3-((5,6-dichloro-2-methyl-4-nitro-1H-benzo[d]imidazol-1-yl)methyl)phenyl)boronic acid,
5,6-dichloro-2-methyl-4-nitro-1-(4-(trifluoro-l4-boranyl)benzyl)-1H-benzo[d]imidazole, or potassium salt thereof,
5,6-dichloro-2-methyl-4-nitro-1-(4-(4,4,5,5-tetramethyl-1,3,2-dioxaborolan-2-yl)benzyl)-1H-benzo[d]imidazole,
2-(4-((5,6-dichloro-2-methyl-4-nitro-1H-benzo[d]imidazol-1-yl)methyl)phenyl)-6-methyl-1,3,6,2-dioxazaborocane-4,8-dione,
(4-(2-(5,6-dichloro-2-methyl-4-nitro-1H-benzo[d]imidazol-1-yl)ethyl)phenyl)boronic acid,
2,5,6-trimethyl-4-nitro-1-(3,4,5-trimethoxybenzyl)-1H-benzo[d]imidazole,
1-((6-chlorobenzo[d][1,3]dioxol-5-yl)methyl)-2,5,6-trimethyl-4-nitro-1H-benzo[d]imidazole,
1-(4-methoxybenzyl)-2,5,6-trimethyl-4-nitro-1H-benzo[d]imidazole,
1-(4-chlorobenzyl)-2,5,6-trimethyl-4-nitro-1H-benzo[d]imidazole,
1-(4-fluorobenzyl)-2,5,6-trimethyl-4-nitro-1H-benzo[d]imidazole,
2,5,6-trimethyl-4-nitro-1-(4-(trifluoromethoxy)benzyl)-1H-benzo[d]imidazole,
1-(4-methoxybenzyl)-5,6-dimethyl-4-nitro-2-(trifluoromethyl)-1H-benzo[d]imidazole,
2,5,6-trimethyl-4-nitro-1-(1-phenylethyl)-1H-benzo[d]imidazole,
(4-((2,5,6-trimethyl-4-nitro-1H-benzo[d]imidazol-1-yl)methyl)phenyl)boronic acid,
(4-((5,6-dimethyl-4-nitro-2-(trifluoromethyl)-1H-benzo[d]imidazol-1-yl)methyl)-phenyl)boronic acid,
1-(4-methoxybenzyl)-4-nitro-1H-benzo[d]imidazole,
(4-((4-nitro-1H-benzo[d]imidazol-1-yl)methyl)phenyl)boronic acid,
(4-((5,5,7,7-tetramethyl-4-nitro-6,7-dihydroindeno[5,6-d]imidazol-1 (5H)-yl)methyl)phenyl)boronic acid,
2-benzyl-1-(4-methoxybenzyl)-7-nitro-1H-benzo[d]imidazole,
2-methoxy-1-(4-methoxybenzyl)-7-nitro-1H-benzo[d]imidazole,
1-(4-methoxybenzyl)-7-nitro-2-phenyl-1H-benzo[d]imidazole, and
(4-((5,6-difluoro-2-methyl-7-nitro-1H-benzo[d]imidazol-1-yl)methyl)phenyl)boronic, acid
   or a pharmaceutically acceptable salt thereof
   and
   a therapeutic agent selected from the group comprising cytarabine, fludarabine, cladribine, clofarabine, nelarabine, capecitabine, floxuridine, deoxycoformycin, azacitidine, decitabine, gemcitabine, sapacitabine, zebularine, fluorouracil and 4′-thio-2′-deoxycytidine.

In a further embodiment, the combination products as defined above, comprises a compound of the invention selected from the group comprising
(4-((5,6-dichloro-2-methyl-4-nitro-1H-benzo[d]imidazol-1-yl)methyl)phenyl)boronic acid,
(4-((5,6-dichloro-2-cyclopropyl-4-nitro-1H-benzo[d]imidazol-1-yl)methyl)phenyl)boronic acid,
(2-((5,6-dichloro-2-methyl-4-nitro-1H-benzo[d]imidazol-1-yl)methyl)phenyl)boronic acid,
(3-((5,6-dichloro-2-methyl-4-nitro-1H-benzo[d]imidazol-1-yl)methyl)phenyl)boronic acid,
5,6-dichloro-2-methyl-4-nitro-1-(4-(trifluoro-l4-boranyl)benzyl)-1H-benzo[d]imidazole, or potassium salt thereof,
5,6-dichloro-2-methyl-4-nitro-1-(4-(4,4,5,5-tetramethyl-1,3,2-dioxaborolan-2-yl)benzyl)-1H-benzo[d]imidazole,
2-(4-((5,6-dichloro-2-methyl-4-nitro-1H-benzo[d]imidazol-1-yl)methyl)phenyl)-6-methyl-1,3,6,2-dioxazaborocane-4,8-dione,
(4-(2-(5,6-dichloro-2-methyl-4-nitro-1H-benzo[d]imidazol-1-yl)ethyl)phenyl)boronic acid,
(4-((2,5,6-trimethyl-4-nitro-1H-benzo[d]imidazol-1-yl)methyl)phenyl)boronic acid, and
(4-((5,6-dimethyl-4-nitro-2-(trifluoromethyl)-1H-benzo[d]imidazol-1-yl)methyl)-phenyl)boronic acid,
   or a pharmaceutically acceptable salt thereof
   and
   a therapeutic agent selected from the group comprising cytarabine, fludarabine, cladribine, clofarabine, nelarabine, capecitabine, floxuridine, deoxycoformycin, azacitidine, decitabine, gemcitabine, sapacitabine, zebularine, fluorouracil and 4′-thio-2′-deoxycytidine.

In a further embodiment, the combination products as defined above, comprises a compound of the invention selected from the group comprising
2-(4-((5,6-dichloro-2-methyl-4-nitro-1H-benzo[d]imidazol-1-yl)methyl)phenyl)-6-methyl-1,3,6,2-dioxazaborocane-4,8-dione,
(4-((5,6-dichloro-2-methyl-4-nitro-1H-benzo[d]imidazol-1-yl)methyl)phenyl)boronic acid,
(4-((5,6-dichloro-2-cyclopropyl-4-nitro-1H-benzo[d]imidazol-1-yl)methyl)phenyl)boronic acid,
5,6-dichloro-2-methyl-4-nitro-1-(4-(4,4,5,5-tetramethyl-1,3,2-dioxaborolan-2-yl)benzyl)-1H-benzo[d]imidazole, and
5,6-dichloro-2-methyl-4-nitro-1-(4-(trifluoro-l4-boranyl)benzyl)-1H-benzo[d]imidazole, or potassium salt thereof,
   or a pharmaceutically acceptable salt thereof, and
   a therapeutic agent selected from the group comprising azacitidine, decitabine and gemcitabine.

In one embodiment, the combination products as defined above, comprises a compound of the invention selected from the group comprising

2-(4-((5,6-dichloro-2-methyl-4-nitro-1H-benzo[d]imidazol-1-yl)methyl)phenyl)-6-methyl-1,3,6,2-dioxazaborocane-4,8-dione, or a pharmaceutically acceptable salt thereof, and
a therapeutic agent selected from azacitidine, decitabine and/or gemcitabine.

In another embodiment, the combination products as defined above, comprises a compound of the invention selected from the group comprising

(4-((5,6-dichloro-2-methyl-4-nitro-1H-benzo[d]imidazol-1-yl)methyl)phenyl)boronic acid, or a pharmaceutically acceptable salt thereof, and
a therapeutic agent selected from azacitidine, decitabine and/or gemcitabine.

In a further embodiment, the combination products as defined above, comprises a compound of the invention selected from the group comprising

(4-((5,6-dichloro-2-cyclopropyl-4-nitro-1H-benzo[d]imidazol-1-yl)methyl)phenyl)-boronic acid, or a pharmaceutically acceptable salt thereof, and
a therapeutic agent selected from azacitidine, decitabine and/or gemcitabine.

In an embodiment, the combination products as defined above, comprises a compound of the invention selected from the group comprising

5,6-dichloro-2-methyl-4-nitro-1-(4-(4,4,5,5-tetramethyl-1,3,2-dioxaborolan-2-yl)benzyl)-1H-benzo[d]imidazole, or a pharmaceutically acceptable salt thereof, and
a therapeutic agent selected from azacitidine, decitabine and/or gemcitabine.

In another embodiment, the combination products as defined above, comprises a compound of the invention selected from the group comprising

5,6-dichloro-2-methyl-4-nitro-1-(4-(trifluoro-l4-boranyl)benzyl)-1H-benzo[d]imidazole, or a pharmaceutically acceptable salt thereof (e.g. a potassium salt), and
a therapeutic agent selected from azacitidine, decitabine and/or gemcitabine.

It is contemplated that any method or composition described herein can be implemented with respect to any other method or composition described herein.

Unless otherwise defined, all technical and scientific terms used herein have the same meaning as commonly understood by one of ordinary skill in the art to which this invention pertains.

### EXAMPLES

### Example 1

Compounds of the invention as described herein may be prepared in accordance with techniques such as those described in the examples provided hereinafter.

The invention is illustrated by way of the following examples, in which the following abbreviations may be employed.
aq aqueous
conc concentrated
DCM dichloromethane
DMF dimethylformamide
DMSO dimethylsulfoxide
EtOAc ethyl acetate
EtOH ethanol
FCC flash column chromatography
h hours
HPLC high pressure liquid chromatography
MeCN acetonitrile
MeOH methanol
min minutes
MTBE methyl tert butyl ether
rt room temperature
TFA trifluoroacetic acid
THF tetrahydrofuran
TLC thin layer chromatography

Starting materials and chemical reagents specified in the syntheses described below are commercially available, e.g. from Sigma-Aldrich, Fine Chemicals Combi-Blocks and other vendors.

Purification of compounds may be carried out using silica-gel column chromatography or preparative reverse phase HPLC (ACE column, acidic gradients with MeCN—H₂O containing 0.1% TFA or XBridge column, basic gradients using MeCN—H₂O containing ammonium bicarbonate) to give the products as their free bases or trifluoroacetic acid salts.

Compounds of general formula 1.3 may be prepared according to Scheme 1.

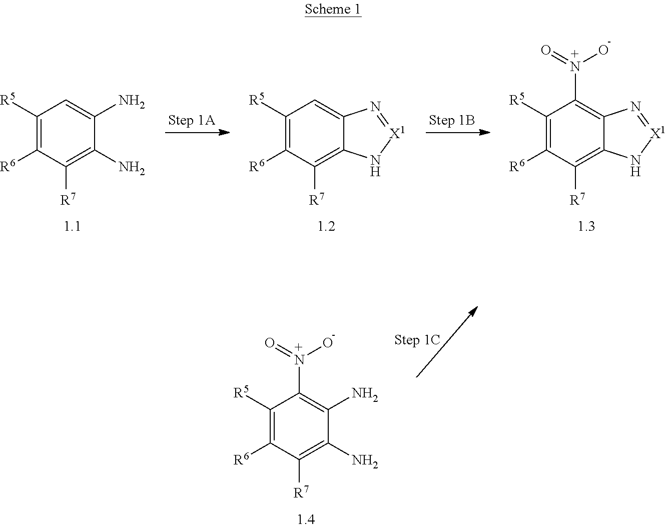

### Step 1A

The appropriate aromatic diamine (1 eq) and carboxylic acid (10 eq) were heated in a sealed tube at 145° C. for 20 min. The mixture was purified by FCC or preparative HPLC to afford the desired compound.

Alternatively, the appropriate aromatic diamine (1 eq) and orthoformate (10 eq) were refluxed for 16 h. The mixture was purified by FCC or preparative HPLC to afford the desired compound.

### Step 1B

65% HNO₃ (1.1 eq) was added dropwise to an appropriately substituted heteroaryl compound (1 eq) in a mixture of MTBE/MeCN (2:1, 0.4 M) at 0° C. The mixture was stirred at 0° C. for 1 h after which the reaction was concentrated in vacuo. The residue was suspended in DCM (0.4 M) and the mixture was added dropwise to ice-cold 95% H₂SO₄ (10 eq). The mixture was allowed to warm to rt and stirred for 16 h. The mixture was poured onto ice-water and neutralized with conc NH₄OH while keeping the temperature below 5° C. The mixture was filtered to afford the desired compound.

### Step 1C

An appropriately substituted aromatic diamine (1 eq) and a carboxylic acid (3 eq) in 4N HCl (0.2 M) were refluxed for 16 h. The mixture was allowed to cool and neutralized with NaHCO₃. The aq layer was extracted with EtOAc, dried over MgSO₄ and concentrated in vacuo to afford the desired compound.

Compounds of general formulas 2.2 and 2.3 may be prepared according to Scheme 2.

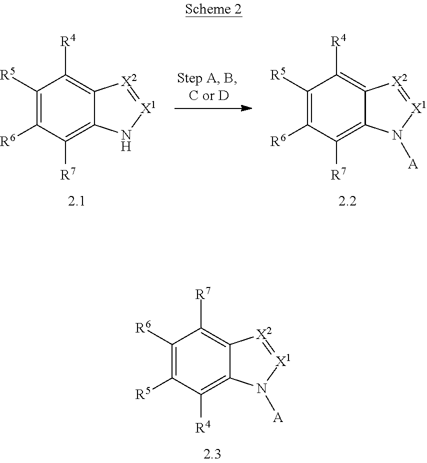

Step 2A—When A=arylalkyl

A mixture of the appropriate alkyl halide (1.5 eq), the appropriate heteroaryl (1 eq), K₂CO₃ (2 eq) and DMSO (0.2 M) was stirred at rt for 16 h. The mixture was allowed to cool and purified by FCC or preparative HPLC to afford the desired compound.

Step 2B—When A=aryl

A mixture of an appropriate heteroaryl (1 eq), an appropriate aromatic boronic acid (1 eq), copper acetate (1 eq), pyridine (2 eq) and DCM (0.1 M) was stirred at rt for four days. The mixture was cooled and purified by FCC or preparative HPLC to afford the desired compound.

Step 2C—When A=arylsulfonyl

A mixture of the appropriate heteroaryl (1 eq), the appropriate arylsulfonyl chloride (1.5 eq), triethylamine (1 eq) and DCM (0.1 M) was stirred at rt for 16 h. The mixture was cooled and purified by FCC or preparative HPLC to afford the desired compound.

Step 2D—When A=arylcarbonyl

A mixture of the appropriate heteroaryl (4.1) (1 eq), the appropriate acid chloride (1.5 eq), triethylamine (1 eq) and DCM (0.1 M) was stirred at rt for 16 h. The mixture was cooled and purified by FCC or preparative HPLC to afford the desired compound.

Compounds of general formula 3.1, 3.2 and 3.3 may be prepared according to Scheme 3.

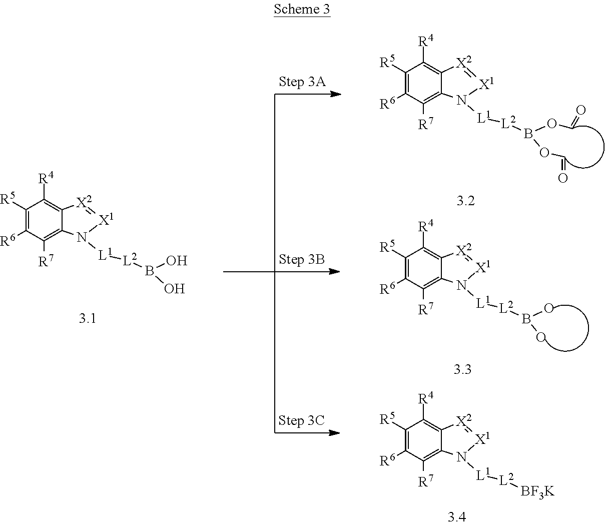

### Step 3A

A mixture of the appropriate boronic acid (1 eq), the appropriate dicarboxylic acid (1.5 eq), MgSO₄ (10 eq) and toluene/DMSO (9:1, 0.05 M) was refluxed for 1 h. The mixture was cooled and purified by FCC or preparative HPLC to afford the desired compound.

### Step 3B

A mixture of the appropriate boronic acid (1 eq), the appropriate diol (2 eq), MgSO₄ (10 eq) and DCM (0.02 M) was stirred at rt for 20 h. The mixture was cooled and purified by FCC or preparative HPLC to afford the desired compound.

### Step 3C

KHF₂ (3.5 eq) in water (4.5 M) was added dropwise to a solution of the appropriate boronic acid (1 eq) in MeOH (0.04 M) and the mixture was stirred at rt for 30 min. The mixture was concentrated in vacuo. The residue was triturated with cold water, filtered and dried to afford the desired compound.

Compounds of general formula 4.3 may be prepared according to Scheme 4.

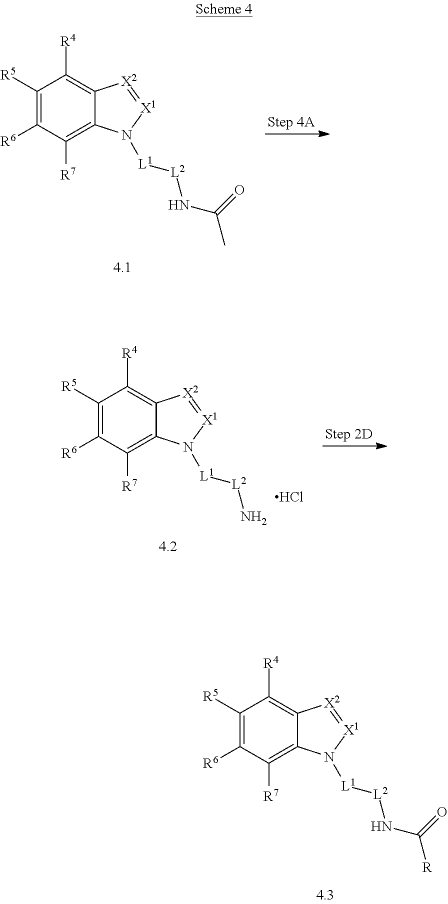

### Step 4A

A mixture of the appropriate acetamide (1 eq) and 6M HCl (0.2 M) was refluxed for 3 h. The mixture was concentrated in vacuo to afford the desired compound as a hydrochloride salt.

Compounds of general formula 5.2 and 5.4 may be prepared according to Scheme 5.

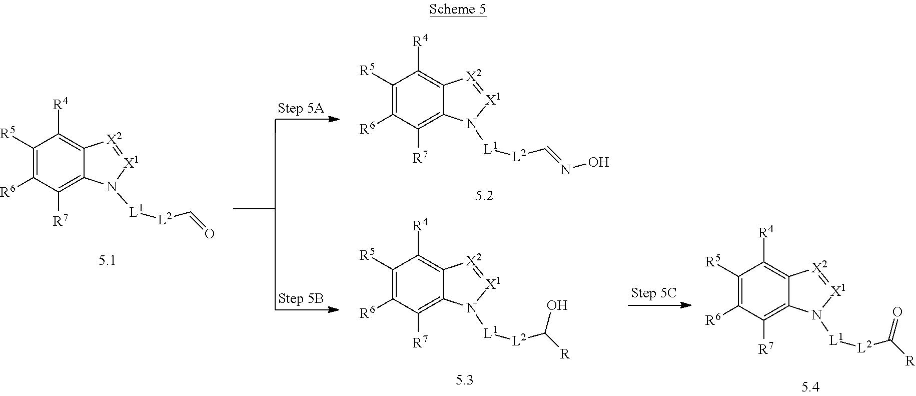

### Step 5A

NaOH (1.5 eq) in water (0.4 M) was added to a stirred mixture of the appropriate aldehyde (1 eq), hydroxylamine hydrochloride (1.5 eq) and EtOH (0.1 M). The mixture was diluted with EtOH (0.05 M) and stirred at rt for 16 h. The mixture was concentrated in vacuo and the residue triturated with cold water. The solids were filtered of and dried to afford the desired compound.

### Step 5B

The appropriate Grignard reagent (1.0 M in THF, 1.1 eq) was added dropwise to a mixture of the appropriate aldehyde (1 eq) and dry THF (0.08 M) at 0° C. The mixture was stirred at this temperature for 10 min and allowed to warm to rt over 4 h. The reaction was quenched by addition of sat aq NH₄Cl, diluted with MeOH and purified by FCC to afford the desired compound.

### Step 5C

A mixture of the appropriate alcohol (1 eq), MnO₂ (5 eq) and 1,4-dioxane (0.07 M) was stirred at 60° C. for 16 h. The mixture was purified by FCC or preparative HPLC to afford the desired compound.

Compounds of general formula 6.2 may be prepared following the procedure in Scheme 6.

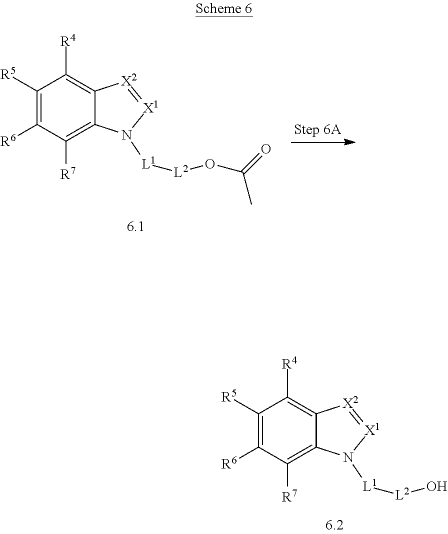

### Step 6A

A solution of LiOH (1.5 eq) in water (1 M) was added to a stirred solution of the appropriate carboxylic ester (1 eq) in 1,4-dioxane (0.06 M) at rt. The mixture was stirred at rt for 16 h and purified by FCC or preparative HPLC to afford the desired compound.

Compounds of general formula 7.2 and 7.3 can be prepared following the procedure in Scheme 7.

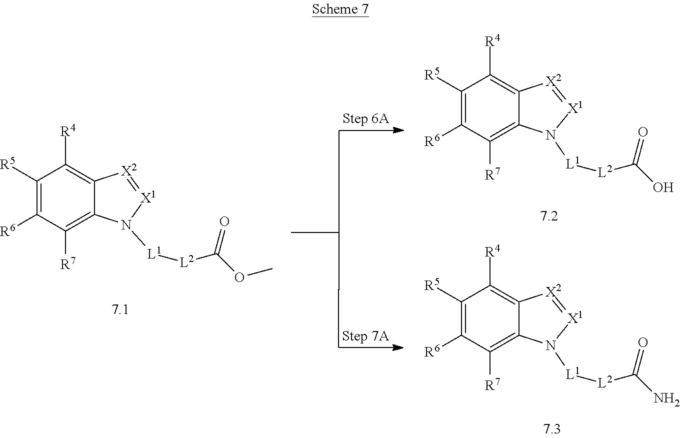

### Step 7A

A mixture of 7 M NH₃ in MeOH (3 eq) and the appropriate carboxylic ester (1 eq) in MeOH (0.1 M) was stirred in a sealed tube at 70° C. for 6 h. Purification by FCC or preparative HPLC to afford the desired compound.

### Biological Example: DCTPP1 Inhibition Assay

DCTPP1 catalyzes the hydrolysis of dCTP to dCMP and PPi. By coupling the reaction to pyrophosphatase added in excess PPi is converted to Pi that can be detected by using the malachite green assay reagent. Briefly, for IC₅₀ value determination the compound to be analysed is diluted in assay buffer in a 1:3 dilution series generating 12 different compound concentrations giving a final DMSO concentration of 1% in the assay well in assay buffer. DCTPP1 diluted in assay buffer (100 mM Tris-acetate, 100 mM KCl, 10 mM magnesium acetate, 1 mM DTT and 0.005% Tween 20) fortified with E. coli pyrophosphatase (0.2 U/mL) is added to a final concentration of 35 nM. dCTP diluted in assay buffer is added to a final concentration of 35 μM. The reaction mixture is incubated with shaking for 20 minutes at 22° C. To 100 μl reaction mixture is added 25 mL Malachite green assay reagent (0.095% Malachite green in 17% H₂SO₄, 1.5% Ammonium molybdate, 0.17% Tween 20) added followed by incubation with shaking for 15 minutes at 22° C. The absorbance of the assay plate is read at 630 nm using a SpectraMax plate reader (Molecular Devices). The IC₅₀ value is determined using a sigmoidal, 4PL (four parameter logistic) plot in GraphPad Prism software.

### Compound Analytical Data and DCTPP1 Inhibition Data

Compounds were synthesised according to the methods described in the schemes presented herein.

IC₅₀ values were determined, as shown in Table 2, whereby the following classification is used:

| | |
|---|---|
| IC₅₀ < 100 nM | A |
| IC₅₀ ≧ 100 nM but < 500 nM | B |
| IC₅₀ ≧ 500 nM but < 1000 nM | C |
| IC₅₀ ≧ 1000 nM | D |
| Not tested | NT |

TABLE 1
Intermediates of general formula 1.2

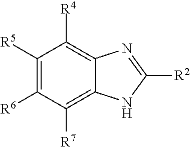

| Inter- | | | | | | | |
|---|---|---|---|---|---|---|---|
| mediate | | | | | | | LCMS |
| ID | R² | R⁴ | R⁵ | R⁶ | R⁷ | ¹H NMR | [M + H]+ |
| 1.2.1 | Me | H | Me | Me | H | (DMSO-d₆) δ: 11.88 (s, 1H), 7.24 (s, 1H), 7.14 (s, 1H), 2.42 (s, 3H), 2.26 (s, 6H) | 161 |
| 1.2.2 | Ph | H | Me | Me | H | — | 223 |
| 1.2.3 | Me | H | Cl | Cl | H | (DMSO-d₆) δ: 7.71 (s, 1H), 2.48 (s, 3H) | 201 |
| 1.2.4 | CF₃ | H | Cl | Cl | H | (DMSO-d₆) δ: 8.03 (s, 1H) | 255 |
| 1.2.5 | cyPr | H | Cl | Cl | H | (DMSO-d₆) δ: 12.62 (br s, 1H), 7.66 (s, 2 H), 2.16-2.06 (m, 1 H), 1.12-1.05 (m, 2 | 227 |
| | | | | | | H), 1.05-0.97 (m, 2 H) | |
| 1.2.6 | Me | H | F | F | H | — | 169 |
| 1.2.7 | Me | H | H | Me | H | (DMSO-d₆) δ: 11.98 (br s, 1 H), 7.34-7.17 (m, 2H), 6.94-6.87 (m, 1 H), 2.43 | 147 |
| | | | | | | (s, 3 H), 2.37 (s, 3H) | |

TABLE 2
Intermediates of general formula 1.3

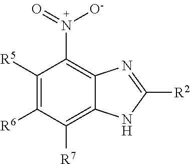

| Inter- | | | | | | |
|---|---|---|---|---|---|---|
| mediate | | | | | | LCMS |
| ID | R² | R⁵ | R⁶ | R⁷ | ¹H NMR | [M + H]+ |
| 1.3.1 | Me | H | H | H | (DMSO-d₆) δ: 13.00 (br s, 1H), 8.05 (dd, J = 8.1, 0.9 Hz, 1H), 7.98 (dd, J = 8.0, 0.9 | 178 |
| | | | | | Hz, 1H), 7.33 (app t, J = 8.0 Hz, 1H), 2.58 (s, 3H) | |
| 1.3.2 | OMe | H | H | H | | 194 |
| 1.3.3 | CH₂OMe | H | H | H | (DMSO-d₆) δ: 13.27 (br s, 1 H), 8.18-8.09 (m, 2 H), 7.44-7.38 (m, 1 H), 4.68 (s, 2 | 208 |
| | | | | | H), 3.37 (s, 3 H) | |
| 1.3.4 | CF₃ | H | H | H | (DMSO-d₆) δ: 14.64 (br s, 1 H), 8.36-8.24 (m, 2 H), 7.59 (app t, J = 8.1 Hz, 1 H) | 232 |
| 1.3.5 | Ph | H | H | H | (DMSO-d₆) δ: 13.25 (br s, 1 H), 8.37-8.32 (m, 2 H), 8.15-8.11 (m, 2 H), 7.63- | 240 |
| | | | | | 7.55 (m, 3 H), 7.44 (app t, J = 8.0 Hz, 1 H) | |
| 1.3.6 | Bn | H | H | H | (DMSO-d₆) δ: 13.23 (br s, 1 H), 8.08 (dd, J = 8.2, 0.9 Hz, 1 H), 8.03 (app d, J = 7.8 | 254 |
| | | | | | Hz, 1 H), 7.40-7.36 (m, 3 H), 7.35-7.29 (m, 2 H), 7.26-7.20 (m, 1 H), 4.31 (s, 2 | |
| | | | | | H) | |
| 1.3.7 | iPr | H | H | H | (DMSO-d₆) δ: 12.93 (br s, 1 H), 8.07 (dd, J = 8.2, 0.9 Hz, 1 H), 8.04 (app d, J = 7.8 | 206 |
| | | | | | Hz, 1 H), 7.35 (app t, J = 8.0 Hz, 1 H), 3.33-3.29 (m, 2 H), 1.36 (d, J = 6.8 Hz, 7 H) | |
| 1.3.8 | Me | F | F | H | (DMSO-d₆) δ: 13.08 (br s, 1H), 8.13 (dd, J = 9.9, 7.1, Hz, 1 H), 2.56 (s, 3 H) | 214 |
| 1.3.9 | Me | Cl | Cl | H | (DMSO-d₆) δ: 13.14 (br s, 1H), 8.10 (s, 1H), 2.56 (s, 3H) | 246 |
| 1.3.10 | cyPr | Cl | Cl | H | (CDCl₃) δ: 10.13 (br s, 1H), 7.99 (s, 1H), 2.05-2.15 (m, 1H), 1.23-1.33 (m, 4H) | 272 |
| 1.3.11 | Me | Me | Me | H | (DMSO-d₆) δ: 12.56 (br s, 1H), 7.73 (s, 0.4H, minor tautomer), 7.47 (s, 0.6H, major | 206 |
| | | | | | tautomer), 2.47-2.49 (m, 4.2H), 2.40 (s, 1.2H, minor tautomer), 2.38 (s, 1.8H, major | |
| | | | | | tautomer), 2.21 (s, 1.8H, major tautomer) | |
| 1.3.12 | CF₃ | Me | Me | H | (DMSO-d₆) δ: 7.77 (s, 1H), 2.45 (s, 3H), 2.33 (s, 3H) | 260 |

TABLE 3
Compounds of general formula 2.2 and 2.3

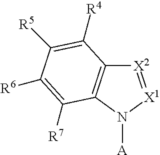

| Com- | | | | | | | | | | | LCMS |
|---|---|---|---|---|---|---|---|---|---|---|---|
| pound | IC₅₀ | | | | | | | | | | [M + |
| ID | (nM) | R² | R⁴ | R⁵ | R⁶ | R⁷ | A | X² | X¹ | ¹H NMR | H]+ |
| 2.2.1 | D | Me | NO₂ | H | H | H | 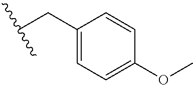 [SMILES: *Cc1ccc(OC)cc1] | N | CR² | (DMSO-d₆) δ: 7.99 (dd, J = 8.0, 0.9 Hz, 1H), 7.98 (dd, J = 8.1, 0.9 Hz, 1H), 7.36 (app t, J = 8.1 Hz, 1H), 7.12 (m, 2H), 6.89 (m, 2H), 5.51 (s, 2H), 3.70 (s, 3H), 2.64 (s, 3H) | 298 |
| 2.2.2 | D | Ph | NO₂ | H | H | H | 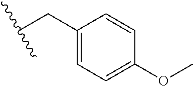 [SMILES: *Cc1ccc(OC)cc1] | N | CR² | — | 360 |
| 2.2.3 | D | CH₂Ph | NO₂ | H | H | H | 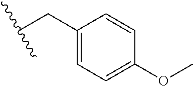 [SMILES: *Cc1ccc(OC)cc1] | N | CR² | — | 374 |
| 2.2.4 | D | CF₃ | NO₂ | H | H | H | 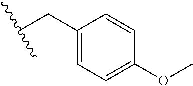 [SMILES: *Cc1ccc(OC)cc1] | N | CR² | — | 352 |
| 2.2.5 | D | OMe | NO₂ | H | H | H | 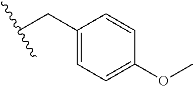 [SMILES: *Cc1ccc(OC)cc1] | N | CR² | — | 314 |
| 2.2.6 | D | CH₂OMe | NO₂ | H | H | H | 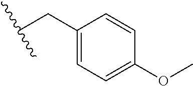 [SMILES: *Cc1ccc(OC)cc1] | N | CR² | — | 328 |
| 2.2.7 | D | iPr | NO₂ | H | H | H | 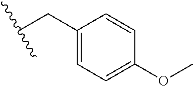 [SMILES: *Cc1ccc(OC)cc1] | N | CR² | — | 326 |
| 2.2.8 | B, 206 | CF₃ | NO₂ | H | H | H | 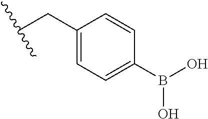 [SMILES: *Cc1ccc(B(O)O)cc1] | N | CR² | (MeOH-d₄) δ: 8.26-8.20 (m, 1H), 7.94-7.92 (m, 1H), 7.70 (br s, 1H), 7.60 (br s, 1H), 7.60- 7.55 (m, 1H), 7.10-7.05 (m, 2H), 5.78 (s, 2H) | 366 |
| 2.2.9 | NT | Me | NO₂ | F | F | H | 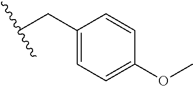 [SMILES: *Cc1ccc(OC)cc1] | N | CR² | — | 334 |
| 2.2.10 | B, 410 | Me | NO₂ | F | F | H | 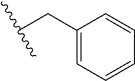 [SMILES: *Cc1ccccc1] | N | CR² | (DMSO-d₆) δ: 8.26 (dd, J = 10.4, 6.6 Hz, 1H), 7.39-7.25 (m, 3H), 7.18-7.12 (m, 2H), 5.56 (s, 2H), 2.56 (s, 3H) | 304 |
| 2.2.11 | D | Me | NO₂ | F | F | H | 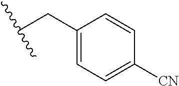 [SMILES: *Cc1ccc(C#N)cc1] | N | CR² | (DMSO-d₆) δ: 8.24 (dd, J = 10.1, 6.6 Hz, 1H), 7.83 (app d, J = 8.3 Hz, 2H), 7.31 (app d, J = 8.3 Hz, 2H), 5.68 (s, 2H), 2.53 (s, 3H) | 329 |
| 2.2.12 | C | Me | NO₂ | F | F | H | 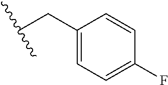 [SMILES: *Cc1ccc(F)cc1] | N | CR² | (DMSO-d₆) δ: 8.27 (dd, J = 10.2, 6.7 Hz, 1H), 7.12-7.27 (m, 4H), 5.55 (s, 2H), 2.56 (s, 3H) | 322 |
| 2.2.13 | D | Me | NO₂ | F | F | H | 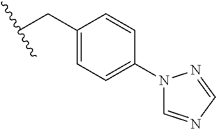 [SMILES: *Cc1ccc(-n2cncn2)cc1] | N | CR² | (DMSO-d₆) δ: 9.27 (s, 1H), 8.29 (dd, J = 10.4, 6.6 Hz, 1H), 8.23 (s, 1H), 7.80-7.86 (m, 2H), 7.33-7.39 (m, 2H), 5.63 (s, 2H), 2.59 (s, 3H) | 371 |
| 2.2.14 | D | Me | NO₂ | F | F | H | 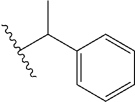 [SMILES: *C(C)c1ccccc1] | N | CR² | (DMSO-d₆) δ: 7.78 (dd, J = 10.5, 6.7 Hz, 1H), 7.27-7.42 (m, 5H), 6.05 (q, J = 7.2 Hz, 1H), 2.59 (s, 3H), 1.93 (d, J = 7.2 Hz, 3H) | 318 |
| 2.2.15 | C | Me | NO₂ | F | F | H | 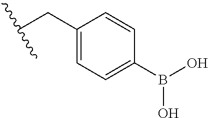 [SMILES: *Cc1ccc(B(O)O)cc1] | N | CR² | (MeOH-d₄) δ: 7.91-8.01 (m, 1H), 7.74-7.62 (m, 2H), 7.19- 7.08 (m, 2H), 5.60 (s, 2H), 2.72 (s, 3H) | 348 |
| 2.2.16 | A | Me | NO₂ | Cl | Cl | H | 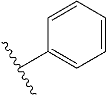 [SMILES: *c1ccccc1] | N | CR² | (CDCl₃) δ: 7.60-7.69 (m, 3H), 7.36 (s, 1H), 7.32-7.36 (m, 2H), 2.54 (s, 3H) | 322 |
| 2.2.17 | A, 74 | Me | NO₂ | Cl | Cl | H | 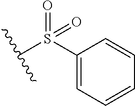 [SMILES: *S(=O)(=O)c1ccccc1] | N | CR² | (CDCl₃) δ: 8.36 (s, 1 H), 7.95- 7.90 (m, 2 H), 7.77-7.71 (m, 1 H), 7.64-7.57 (m, 2 H), 2.81 (s, 3 H) | 386 |
| 2.2.18 | B | Me | NO₂ | Cl | Cl | H | 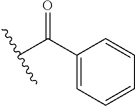 [SMILES: *C(=O)c1ccccc1] | N | CR² | (CDCl₃) δ: 7.82-7.72 (m, 3 H), 7.64-7.58 (m, 2 H), 7.28 (s, 1 H), 2.65 (s, 3 H) | 350 |
| 2.2.19 | A | Me | NO₂ | Cl | Cl | H | 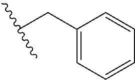 [SMILES: *Cc1ccccc1] | N | CR² | (DMSO-d₆) δ: 8.31 (s, 1H), 7.28- 7.39 (m, 3H), 7.16 (m, 2H), 5.59 (s, 2H), 2.55 (s, 3H) | 336 |
| 2.2.20 | A, 41 | Me | NO₂ | Cl | Cl | H | 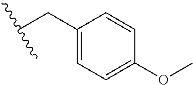 [SMILES: *Cc1ccc(OC)cc1] | N | CR² | (CDCl₃) δ: 7.44 (s, 1 H), 6.96 (d, J = 9.0 Hz, 2 H), 6.85 (d, J = 8.8 Hz, 2 H), 3.77 (s, 3 H), 2.59 (s, 3 H) | 366 |
| 2.2.21 | B | Me | NO₂ | Cl | Cl | H | 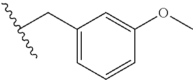 [SMILES: *Cc1cccc(OC)c1] | N | CR² | (DMSO-d₆) δ: 8.31 (s, 1H), 7.26 (app t, J = 8.0 Hz, 1H), 6.88 (m, 1H), 6.79 (m, 1H), 6.63 (m, 1H), 5.55 (s, 2H), 3.72 (s, 3H), 2.55 (s, 3H) | 366 |
| 2.2.22 | A | Me | NO₂ | Cl | Cl | H | 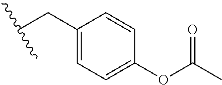 [SMILES: *Cc1ccc(OC(C)=O)cc1] | N | CR² | (CDCl₃) δ: 7.46 (s, 1 H), 7.12- 7.07 (m, 2 H), 7.05-7.00 (m, 2 H), 5.32 (s, 2 H), 2.62 (s, 3 H), 2.30 (s, 3 H) | 394 |
| 2.2.23 | NT | Me | NO₂ | Cl | Cl | H | 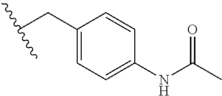 [SMILES: *Cc1ccc(NC(C)=O)cc1] | N | CR² | — | 393 |
| 2.2.24 | NT | Me | NO₂ | Cl | Cl | H | 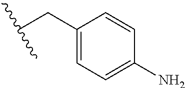 [SMILES: *Cc1ccc(N)cc1] | N | CR² | — | 351 |
| 2.2.25 | A | Me | NO₂ | Cl | Cl | H | 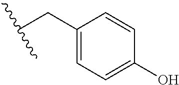 [SMILES: *Cc1ccc(O)cc1] | N | CR² | (DMSO-d₆) δ: 9.48 (s, 1 H), 8.29 (s, 1 H), 7.08-6.99 (m, 2 H), 6.76-6.66 (m, 2 H), 5.44 (s, 2 H), 2.56 (s, 3 H) | 352 |
| 2.2.26 | A | Me | NO₂ | Cl | Cl | H | 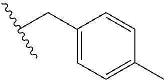 [SMILES: *Cc1ccc(C)cc1] | N | CR² | (DMSO-d₆) δ: 8.30 (s, 1H), 7.16 (m, 2H), 7.06 (m, 2H), 5.53 (s, 2H), 2.55 (s, 3H), 2.26 (s, 3H) | 350 |
| 2.2.27 | B | Me | NO₂ | Cl | Cl | H | 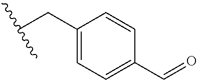 [SMILES: *Cc1ccc(C=O)cc1] | N | CR² | (CDCl₃) δ: 10.01 (s, 1H), 7.89 (app d, J = 8.03 Hz, 2H), 7.43 (s, 1H), 7.18 (app d, J = 8.03 Hz, 2H), 5.41 (s, 2H), 2.62 (s, 3H) | 364 |
| 2.2.28 | A | Me | NO₂ | Cl | Cl | H | 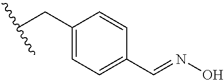 [SMILES: *Cc1ccc(/C=N/O)cc1] | N | CR² | DMSO-d₆) δ: 11.25 (s, 1H), 8.31 (s, 1H), 8.12 (s, 1H), 7.56-7.60 (m, 2H), 7.17-7.21 (m, 2H), 5.61 (s, 2H), 2.56 (s, 3H) | 379 |
| 2.2.29 | NT | Me | NO₂ | Cl | Cl | H | 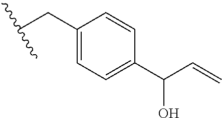 [SMILES: *Cc1ccc(C(O)C=C)cc1] | N | CR² | — | 392 |
| 2.2.30 | A | Me | NO₂ | Cl | Cl | H | 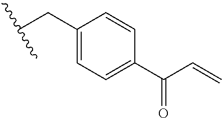 [SMILES: *Cc1ccc(C(=O)C=C)cc1] | N | CR² | (CDCl₃) δ: 7.98-7.92 (m, 2 H), 7.45 (s, 1 H), 7.15- 7.13 (m, 2H), 7.11 (dd, J = 17.2, 10.6 Hz, 1 H), 6.45 (dd, J = 17.2, 1.5 Hz, 1 H), 5.98 (dd, J = 10.6, 1.5 Hz, 1 H), 5.40 (s, 2 H), 2.63 (s, 3 H) | 390 |
| 2.2.31 | NT | Me | NO₂ | Cl | Cl | H | 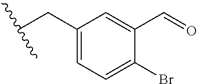 [SMILES: *Cc1ccc(Br)c(C=O)c1] | N | CR² | — | 442 |
| 2.2.32 | A | Me | NO₂ | Cl | Cl | H | 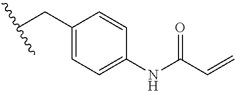 [SMILES: *Cc1ccc(NC(=O)C=C)cc1] | N | CR² | (DMSO-d₆) δ: 10.21 (s, 1 H), 8.31 (s, 1 H), 7.64 (app d, J = 8.3 Hz, 2H), 7.16 (app d, J = 8.3 Hz, 2H), 6.42 (dd, J = 16.9, 10.1 Hz, 1 H), 6.23 (dd, J = 16.9, 1.8 Hz, 1 H), 5.74 (dd, J = | 405 |
| | | | | | | | | | | 10.1, 1.8 Hz, 1 H), 5.53 (s, 2 H), | |
| | | | | | | | | | | 2.56 (s, 3 H) | |
| 2.2.33 | B | Me | NO₂ | Cl | Cl | H | 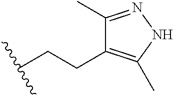 [SMILES: *CCc1c(C)n[nH]c1C] | N | CR² | (DMSO-d₆) δ: 11.92 (br s, 1H), 8.01 (s, 1H), 4.34 (t, J = 6.2 Hz, 1H), 2.73 (t, J = 6.2 Hz, 2H), 2.23 (s, 3H), 1.55-1.85 (br s, 6H) | 368 |
| 2.2.34 | A | Me | NO₂ | Cl | Cl | H | 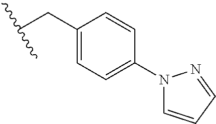 [SMILES: *Cc1ccc(-n2cccn2)cc1] | N | CR² | (CDCl₃) δ: 7.90 (app d, J = 2.5 Hz, 1H), 7.68-7.73 (m, 3H), 7.46 (app s, 1H), 7.12 (d, J = 8.8 Hz, 2H), 6.45-6.50 (m, 1H), 5.35 (s, 2H), 2.63 (s, 3H) | 402 |
| 2.2.35 | C | Me | NO₂ | Cl | Cl | H | 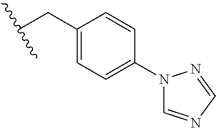 [SMILES: *Cc1ccc(-n2cncn2)cc1] | N | CR² | (DMSO-d₆) δ: 9.27 (s, 1H), 8.35 (s, 1H), 8.22 (s, 1H), 7.84 (m, 2H), 7.37 (m, 2H), 5.66 (s, 2H), 2.58 (s, 3H) | 403 |
| 2.2.36 | B | Me | NO₂ | Cl | Cl | H | 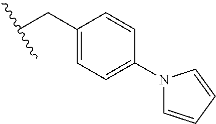 [SMILES: *Cc1ccc(-n2cccc2)cc1] | N | CR² | (DMSO-d₆) δ: 8.33 (s, 1H), 7.50- 7.56 (m, 2H), 7.40 (app t, J = 7.8 Hz, 1H), 7.35 (app t, J = 2.2 Hz, 2H), 6.89 (app d, J = 7.5 Hz, 1H), 6.27 (app t, J = 2.2 Hz, 2H), 5.62 (s, 2H), 4.07-4.49 (m, 1H), 2.59 (s, 3H) | 401 |
| 2.2.37 | A | Me | NO₂ | Cl | Cl | H | 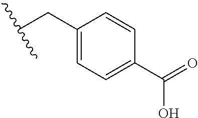 [SMILES: *Cc1ccc(C(=O)O)cc1] | N | CR² | (DMSO-d₆) δ: 12.98 (br s, 1 H), 8.31 (s, 1 H), 7.94-7.89 (app d, J = 8.3 Hz, 2H), 7.28-7.21 (app d, J = 8.3 Hz, 2 H), 5.68 (s, 2 H), 2.53 (s, 3 H) | 380 |
| 2.2.38 | B | Me | NO₂ | Cl | Cl | H | 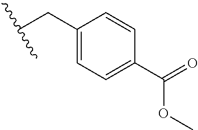 [SMILES: *Cc1ccc(C(=O)OC)cc1] | N | CR² | (CDCl₃) δ: 8.03 (app d, J = 8.3 Hz, 2 H), 7.43 (s, 1 H), 7.08 (app d, J = 8.3 Hz, 2 H), 5.38 (s, 2H), 3.92 (s, 3 H), 2.61 (s, 3 H) | 394 |
| 2.2.39 | A | Me | NO₂ | Cl | Cl | H | 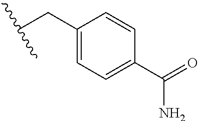 [SMILES: *Cc1ccc(C(N)=O)cc1] | N | CR² | — | 379 |
| 2.2.40 | B | Me | NO₂ | Cl | Cl | H | 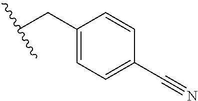 [SMILES: *Cc1ccc(C#N)cc1] | N | CR² | (DMSO-d₆) δ: 8.31 (s, 1H), 7.78- 7.85 (app d, J = 8.3 Hz, 2H), 7.28-7.34 (app d, J = 8.3 Hz, 2H), 5.71 (s, 2H), 2.53 (s, 3H) | 361 |
| 2.2.41 | B | Me | NO₂ | Cl | Cl | H | 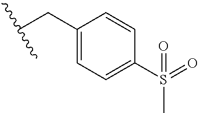 [SMILES: *Cc1ccc(S(C)(=O)=O)cc1] | N | CR² | — | 414 |
| 2.2.42 | B | Me | NO₂ | Cl | Cl | H | 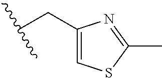 [SMILES: *Cc1csc(C)n1] | N | CR² | (CDCl₃) δ: 7.64 (s, 1 H), 6.83 (s, 1 H), 5.34 (s, 2 H), 2.74 (s, 3 H), 2.67 (s, 3 H) | 357 |
| 2.2.43 | B | Me | NO₂ | Cl | Cl | H | 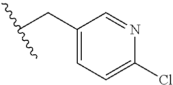 [SMILES: *Cc1ccc(Cl)nc1] | N | CR² | (CDCl₃) δ: 8.30 (d, J = 2.3 Hz, 1 H), 7.45 (s, 1 H), 7.33 (d, J = 8.0 Hz, 1 H), 7.21 (dd, J = 2.3, 8.0 Hz, 1 H), 5.34 (s, 2 H), 2.64 (s, 3 H) | 371 |
| 2.2.44 | A, 47 | Me | NO₂ | Cl | Cl | H | 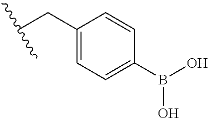 [SMILES: *Cc1ccc(B(O)O)cc1] | N | CR² | (MeOH-d₄) δ: 7.88 (s, 1 H), 7.58 (d, J = 7.8 Hz, 2 H), 7.09 (d, J = 7.8 Hz, 2 H), 5.50 (s, 2 H), 2.55 (s, 3 H) | 380 |
| 2.2.45 | A, 27 | cyclo- propyl | NO₂ | Cl | Cl | H | 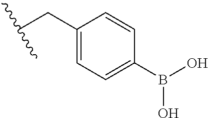 [SMILES: *Cc1ccc(B(O)O)cc1] | N | CR² | (Acetone-d₆) δ: 8.01 (s, 1H), 7.85-7.89 (m, 2H), 7.24-7.27 (m, 2H), 5.77 (s, 2H), 2.27- 2.31 (m, 1H), 1.14-1.19 (m, 4H) | 406 |
| 2.2.46 | A | Me | NO₂ | Cl | Cl | H | 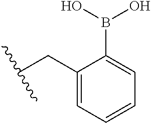 [SMILES: *Cc1ccccc1B(O)O] | N | CR² | (MeOH-d₄) δ: 7.74 (s, 1 H), 7.45-7.39 (m, 1 H), 7.39-7.34 (m, 2 H), 7.10-7.05 (m, 1 H), 5.52 (s, 2 H), 2.55 (s, 3 H) | 380 |
| 2.2.47 | A | Me | NO₂ | Cl | Cl | H | 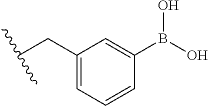 [SMILES: *Cc1cccc(B(O)O)c1] | N | CR² | (MeOH-d₄) δ: 7.93 (s, 1 H), 7.57-7.54 (m, 1 H), 7.43-7.31 (m,2 H), 7.16-7.14 (m, 1 H), 5.53 (s, 2 H), 2.60 (s, 3 H) | 380 |
| 2.2.48 | A, 39 | Me | NO₂ | Cl | Cl | H | 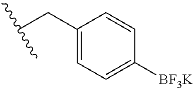 | N | CR² | (MeOH-d₄) δ: 7.85 (s, 1 H), 7.45 (app d, J = 7.8 Hz, 2H), 6.93 (app d, J = 7.8 Hz, 2 H), 5.39 (s, 2 H), 2.55 (s, 3 H) | 380 |
| 2.2.49 | A, 30 | Me | NO₂ | Cl | Cl | H | 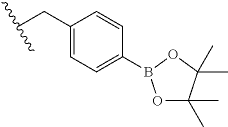 [SMILES: *Cc1ccc(B2OC(C)(C)C(C)(C)O2)cc1] | N | CR² | (CDCl₃) δ: 7.82-7.77 (m, 2 H), 7.44 (s, 1 H), 7.04-7.00 (m, , 2 H), 5.34 (s, 2 H), 2.62 (s, 3 H), 1.34 (s, 12 H) | 462 |
| 2.2.50 | A, 44 | Me | NO₂ | Cl | Cl | H | 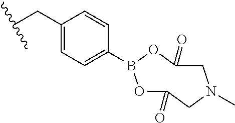 [SMILES: *Cc1ccc(B2OC(=O)CN(C)CC(=O)O2)cc1] | N | CR² | (Acetone-d₆) δ: 8.01 (s, 1 H), 7.54 (app d, J = 8.1 Hz, 2 H), 7.24 (app d, J = 8.3 Hz, 2 H), 5.65 (s, 2H), 4.34 (d, J = 16.9 Hz, 2 H), 4.13 (d, J = 16.9 Hz, 2 H), 2.60 (s, 3 H) | 491 |
| 2.2.51 | B, 205 | Me | NO₂ | Cl | Cl | H | 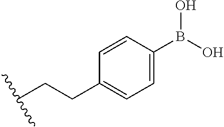 [SMILES: *CCc1ccc(B(O)O)cc1] | N | CR² | (MeOH-d₄) δ: 7.74 (s, 1H), 7.35- 7.64 (m, 2H), 6.92 (app d, J = 7.6 Hz, 2H), 4.49 (t, J = 6.3 Hz, 2H), 3.09 (t, J = 6.3 Hz, 2H), 2.20 (br. s., 3H) | 395 |
| 2.2.52 | D | Me | NO₂ | Me | Me | H | 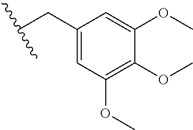 [SMILES: *Cc1cc(OC)c(OC)c(OC)c1] | N | CR² | (DMSO-d₆) δ: 7.66 (s, 1H), 6.50 (s, 2H), 5.38 (s, 2H), 3.62 (s, 3H), 2.57 (s, 3H), 2.38 (s, 3H), 2.22 (s, 3H) | 386 |
| 2.2.53 | D | Me | NO₂ | Me | Me | H | 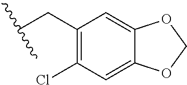 [SMILES: *Cc1cc2c(cc1Cl)OCO2] | N | CR² | ¹H NMR (DMSO-d₆) δ: 7.50 (s, 1H), 7.19 (s, 1H), 6.15 (s, 1H), 6.04 (s, 2H), 5.43 (s, 2H) | 374 |
| 2.2.54 | A | Me | NO₂ | Me | Me | H | 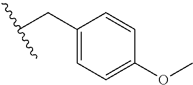 [SMILES: *Cc1ccc(OC)cc1] | N | CR² | (DMSO-d₆) δ: 7.63 (s, 1H), 7.09 (m, 2H), 6.89 (m, 2H), 6.58 (m, 2H), 5.41 (s, 2H), 3.70 (s, 3H), 2.51 (s, 3H), 2.37 (s, 3H), 2.22 (s, 3H) | 326 |
| 2.2.55 | A | Me | NO₂ | Me | Me | H | 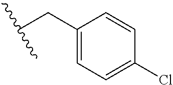 [SMILES: *Cc1ccc(Cl)cc1] | N | CR² | (DMSO-d₆) δ: 7.61 (s, 1H), 7.40 (app d, J = 8.6 Hz, 2H), 7.13 (app d, J = 8.6 Hz, 2H), 5.50 (s, 2H), 2.50 (s, 3H), 2.36 (s, 3H), 2.22 (s, 3H) | 330 |
| 2.2.56 | B | Me | NO₂ | Me | Me | H | 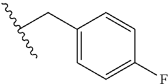 [SMILES: *Cc1ccc(F)cc1] | N | CR² | (DMSO-d₆) δ: 7.62 (s, 1H), 7.35 (m, 2H), 7.23 (m, 2H), 5.55/s, 2H), 2.51 (s, 3H), 2.37 (s, 3H), 2.23 (s, 3H) | 314 |
| 2.2.57 | B | Me | NO₂ | Me | Me | H | 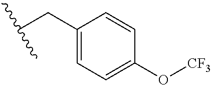 [SMILES: *Cc1ccc(OC(F)(F)F)cc1] | N | CR² | (DMSO-d₆) δ: 7.62 (s, 1H), 7.35 (m, 2H), 7.23 (m, 2H), 5.55 (s, 2H), 2.51 (s, 3H), 2.37 (s, 3H), 2.23 (s, 3H) | 380 |
| 2.2.58 | A, 14 | CF₃ | NO₂ | Me | Me | H | 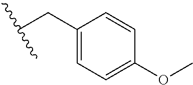 [SMILES: *Cc1ccc(OC)cc1] | N | CR² | (CDCl₃) δ: 7.23 (s, 1H), 7.02- 7.07 (m, 2H), 6.83-6.89 (m, 2H), 5.47 (s, 2H), 3.80 (s, 3H), 2.41 (s, 3H), 2.36 (s, 3H) | 380 |
| 2.2.59 | B | Me | NO₂ | Me | Me | H | 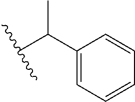 [SMILES: *C(C)c1ccccc1] | N | CR² | (DMSO-d₆) δ: 7.24-7.40 (m, 6H), 5.95 (q, J = 7.2 Hz, 1H), 2.55 (s, 3H), 2.27 (s, 3H), 2.18 (s, 3H), 1.93 (d, J = 7.2 Hz, 3H) | 310 |
| 2.2.60 | A, 65 | Me | NO₂ | Me | Me | H | 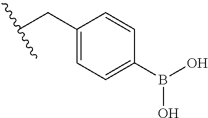 [SMILES: *Cc1ccc(B(O)O)cc1] | N | CR² | (MeOH-d₄) δ: 7.74 (s, 1H), 7.68- 7.57 (m, 2H), 7.26-7.12 (m, 2H), 5.64 (s, 2H), 2.77 (s, 3H), 2.48 (s, 3H), 2.47 (s, 3H) | 340 |
| 2.2.61 | A | CF₃ | NO₂ | Me | Me | H | 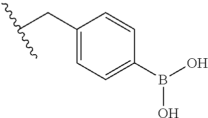 [SMILES: *Cc1ccc(B(O)O)cc1] | N | CR² | (MeOH-d₄) δ: 7.70 (s, 1 H), 7.63-7.46 (m, 2 H), 7.12-6.97 (m, 2 H), 5.66 (s, 2 H), 2.39 (s, 3 H), 2.33 (s, 3 H) | 394 |
| 2.2.62 | D | H | NO₂ | H | H | H | 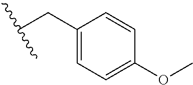 [SMILES: *Cc1ccc(OC)cc1] | CH | CR² | — | 283 |
| 2.2.63 | D | H | NO₂ | H | H | H | 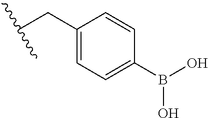 [SMILES: *Cc1ccc(B(O)O)cc1] | CH | CR² | (CDCl₃) δ: 8.08 (dd, J = 8.1, 0.8 Hz, 1 H), 7.78 (app d, J = 8.1 Hz, 1 H), 7.64 (d, J = 3.1 Hz, 1 H), 7.55 (app d, J = 7.8 Hz, 2 H), 7.25 (app t, J = 8.1 Hz, 1 H), 7.19 (dd, J = 0.8, 3.1 Hz, 1 H), 7.14 (app d, J = 7.8 Hz, 2 H), 5.51 (s, 2 H) | 298 |
| 2.2.64 | D | — | NO₂ | 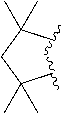 [SMILES: *C(C)(C)CC(*)(C)C] | | H | 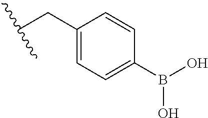 [SMILES: *Cc1ccc(B(O)O)cc1] | CH | N | (DMSO-d₆) δ: 8.67 (d, J = 0.8 Hz, 1H), 8.11 (s, 1H), 7.83 (d, J = 0.8 Hz, 1H), 7.74 (app d, J = 8.2 Hz, 2H), 7.28 (app d, J = 8.2 Hz, 4H), 5.67 (s, 2H), 2.01 (s, 2H), 1.46 (s, 6H), 1.37 (s, 6H) | 394 |
| 2.3.1 | D | CH₂Ph | H | H | H | NO₂ | 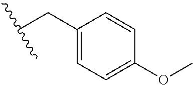 [SMILES: *Cc1ccc(OC)cc1] | N | CR² | (DMSO-d₆) δ: 8.01 (dd, J = 8.1, 1.0 Hz, 1 H), 7.87 (dd, J = 8.1, 1.0 Hz, 1 H), 7.36 (app t, J = 8.1 Hz, 1 H), 7.31-7.20 (m, 5 H), 6.98-6.93 (m, 2 H), 6.83- 6.78 (m, 2 H), 5.51 (s, 2 H), | 374 |
| | | | | | | | | | | 4.43 (s, 2 H), 3.68 (s, 3 H) | |
| 2.3.2 | D | OMe | H | H | H | NO₂ | 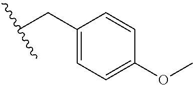 [SMILES: *Cc1ccc(OC)cc1] | N | CR² | — | 314 |
| 2.3.3 | D | Ph | H | H | H | NO₂ | 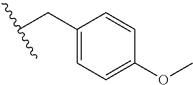 [SMILES: *Cc1ccc(OC)cc1] | N | CR² | — | 360 |
| 2.3.4 | C | Me | H | F | F | NO₂ | 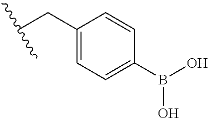 [SMILES: *Cc1ccc(B(O)O)cc1] | N | CR² | (MeOH-d₄) δ: 7.90-7.76 (s, 1H), 7.73-7.46 (m, 2H), 6.96- 6.79 (m, 2H), 5.46 (s, 2H), 2.67 (s, 3H) | 348 |

### Example 2: Cancer Cell Viability Assay and Synergy Assay

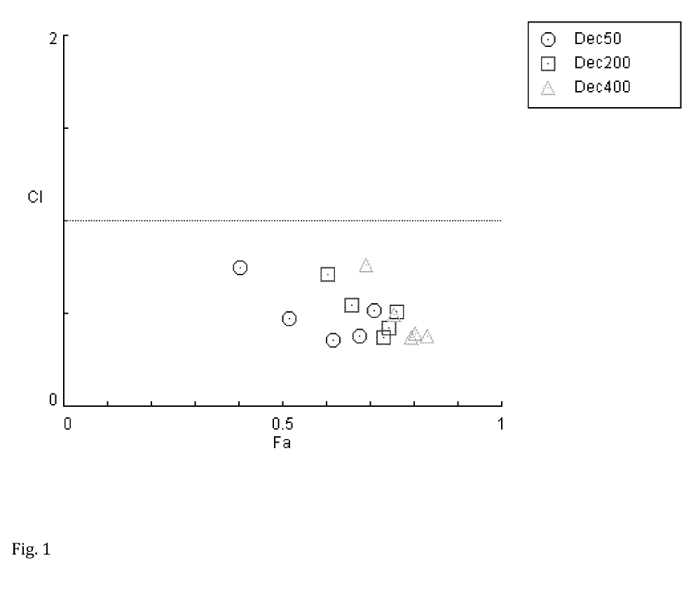

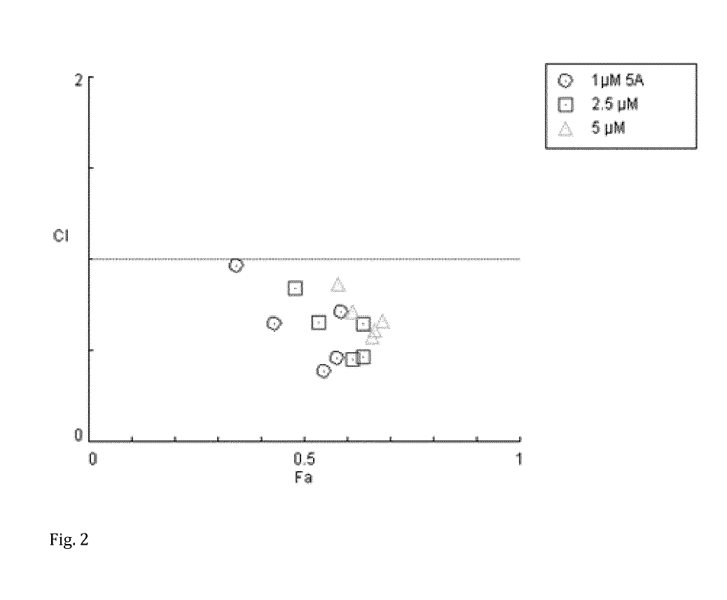

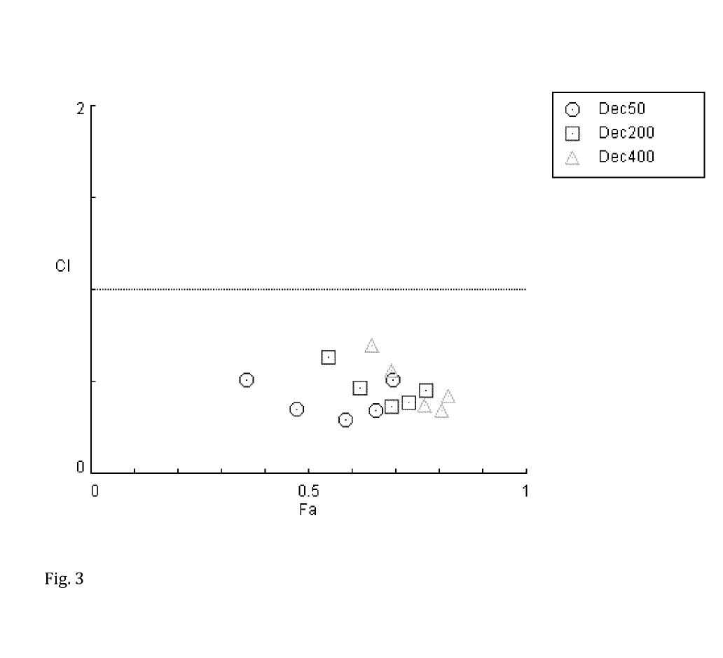

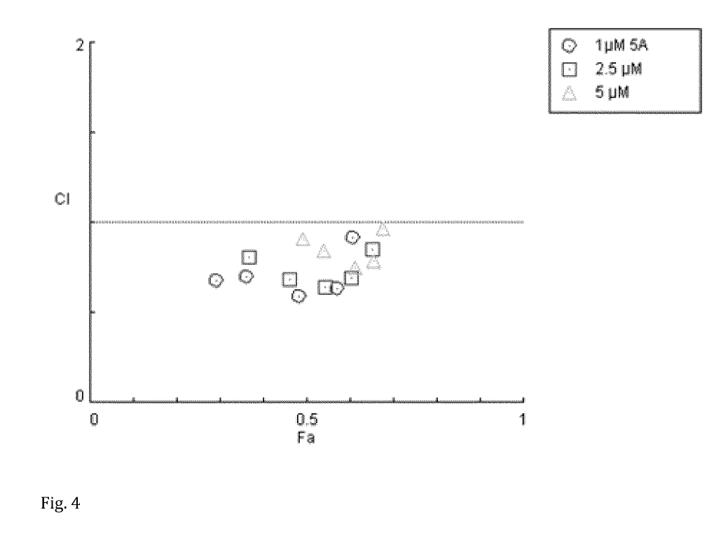

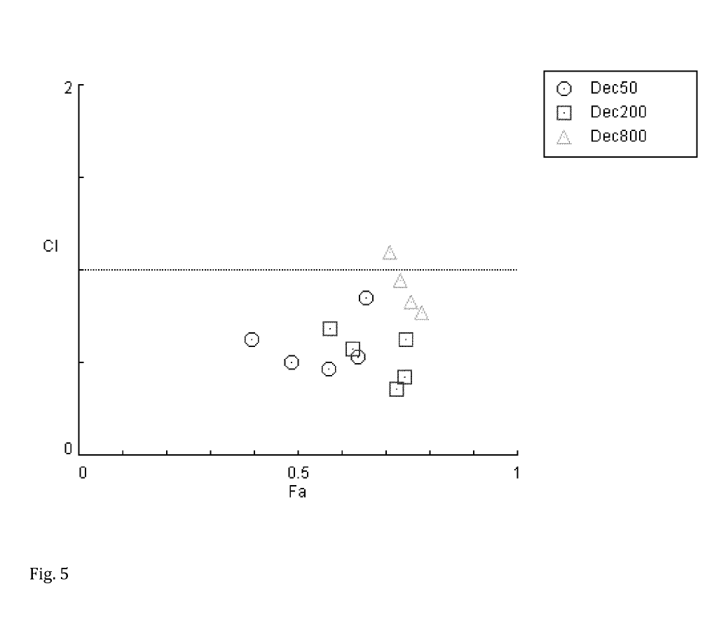

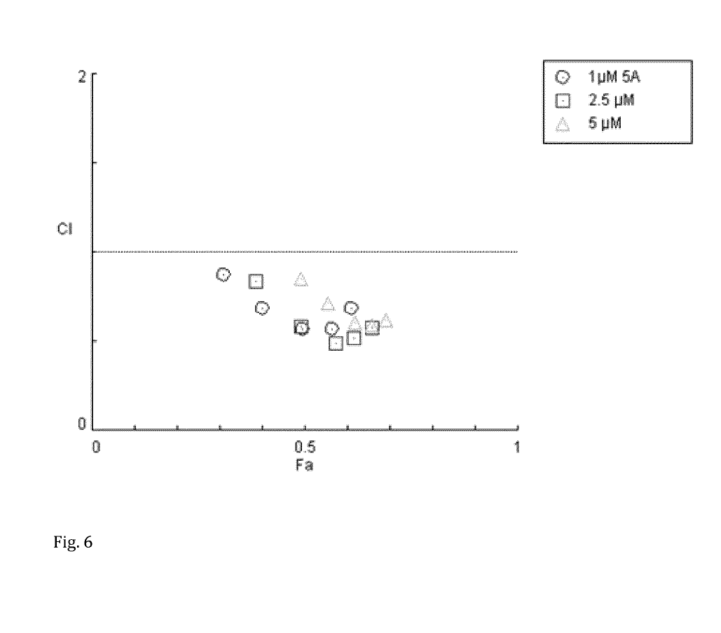

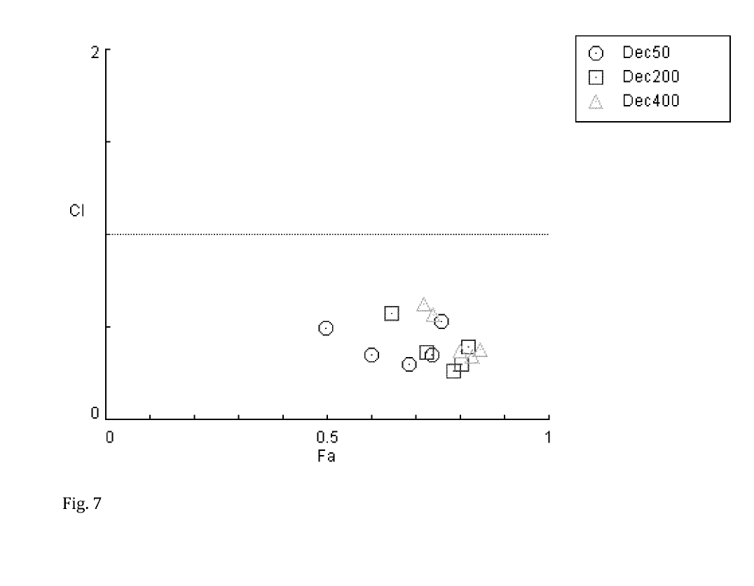

Compounds can be screened for their ability to reduce HL60 cancer cell viability alone and/or in combination with cytidine analogues (concentration range) using a 72 h cell viability assay (resazurin assay, Nature 508, 215-221). HL60 cells were grown in RPMI-Glutamax (Lifetechnologies, cat. Nr. 61870-010), containing 10% fetal bovine serum (FBS), penicillin (50 U/ml) and streptomycin (50 μg/ml). Cells were maintained at 37° C. in a 5% CO₂ atmosphere. Synergistic effect can be quantified using the Combination Index (using Compusyn, according to Cancer Res. 2010, 70, 440-446). Compounds from the invention show a synergistic effect (Combination Index <0.8) with cytidine analogues such as decitabine (Dec) and 5-azacytidine (5A), decreasing the viability of HL60 cancer cells (see FIGS. 1, 2, 3, 4, 5, 6 and 7).

### Example 3: Activated Immune System Cell Viability Assay

Compounds can be screened for their ability to reduce cell viability of activated cells from the immune system using a 24 h cell viability assay. Neutrophils, eosinophils and monocytes can be isolated from whole blood using commercial isolation kits (Miltenyi Biotec). Cells can be activated with phorbol-12-myristate-13-acetate (PMA) (200 nM) and treated with a range of concentration of the compounds of interest. Cell viability can be quantified using a MTT cell viability assay (Int Arch Allergy Appl Immunol. 1990; 92(2):189-92).

1. A method of treatment of a proliferative disorder, comprising administering, to a patient in need of such treatment, a therapeutically effective amount of a compound of formula I, 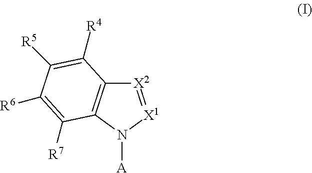
or a pharmaceutically acceptable salt thereof,
wherein:
A represents -L¹-L²-L³-A¹;
A¹ represents aryl optionally substituted by one or more groups independently selected from Y¹, or heteroaryl optionally substituted by one or more groups independently selected from Y²;
each one of L¹ and L³ independently represents a single bond or C₁₋₃ alkylene optionally substituted by one or more halo;
L² represents a single bond, —C(Q)-, —N(R¹)—, —O— or —S(O)ₙ—;
X¹ represents C(R²);
X² represents N;
R¹ represents H or C₁₋₆ alkyl optionally substituted by one or more halo;
R² represents H, R^{a} or —OR^{b};
R⁴ represents —NO₂;
R⁷ represents H;
R⁵ and R⁶ independently represent H, halo, —CN, R^{c}, —N₃ or —NO₂;
Q represents ═O or ═S;
R^{a} represents C₁₋₆ alkyl optionally substituted by one or more groups independently selected from D¹, or phenyl optionally substituted by one or two groups independently selected from D²;
R^{b} represents H or C₁₋₆ alkyl optionally substituted by one or more F;
D¹ represents F, —OC₁₋₄ alkyl optionally substituted by one or more F, or phenyl optionally substituted by one or two groups independently selected from D²; and
D² represents F, Cl, C₁₋₄ alkyl optionally substituted by one or more F or —OC₁₋₃alkyl optionally substituted by one or more F;
each R^{c} independently represents C₁₋₆alkyl optionally substituted by one or more F;
D¹ represents halo, —OC₁₋₆ alkyl optionally substituted by one or more halo, aryl optionally substituted by one or more groups independently selected from D² or heteroaryl optionally substituted by one or more groups independently selected from D³;
each D² and D³ independently represents halo, C₁₋₆ alkyl optionally substituted by one or more halo or —OC₁₋₆ alkyl optionally substituted by one or more halo;
each Y¹ and Y² independently represents halo, —BF₃M, —B(OR^{a1})₂, R^{b1}, —CN, —C(Q²)R^{c1}, —C(Q²)OR^{d1}, —C(Q²)N(R^{e1})R^{f1}, —N₃, —NO₂, —N(R^{g1})R^{h1}, —N(Rⁱ¹)C(Q²)R^{j1}, —N(R^{k1})C(Q²)N(R^{l1})R^{m1}, —N(Rⁿ¹)C(Q²)OR^{o1}, —N(R^{p1})S(O)ₙR^{q1}, —N(R^{r1})S(O)ₙN(R^{s1})R^{t1}, —OR^{u1}, —OC(Q²)R^{v1}, —OC(Q²)N(R^{w1})R^{x1}, —OC(Q²)OR^{y1}, —OS(O)ₙR^{z1}, —SR^{aa1}, —S(O)ₙR^{ab1}, —S(O)ₙN(R^{ac1})R^{ad1}, heterocycloalkyl optionally substituted by one or more groups independently selected from Z¹, aryl substituted by one or more groups independently selected from Z², heteroaryl optionally substituted by one or more groups independently selected from Z³ or Q²;
each Z¹ independently represents halo, R^{b1}, —CN, —C(Q²)R^{c1}, —C(Q²)OR^{d1}, —C(Q²)N(R^{e1})R^{f1}, —N₃, —NO₂, —N(R^{g1})R^{h1}, —N(Rⁱ¹)C(Q²)R^{j1}, —N(R^{k1})C(Q²)N(R^{l1})R^{m1}, —N(Rⁿ¹)C(Q²)OR^{o1}, —N(R^{p1})S(O)ₙR^{q1}, —N(R^{r1})S(O)ₙN(R^{s1})R^{t1}, —OR^{u1}, —OC(Q²)R^{v1}, —OC(Q²)N(R^{w1})R^{x1}, —OC(Q²)OR^{y1}, —OS(O)ₙR^{z1}, —SR^{aa1}, —S(O)ₙR^{ab1}, —S(O)ₙN(R^{ac1})R^{ad1} or Q²;
each Z² and Z³ independently represents halo, —BF₃M, —B(OR^{a1})₂, R^{b1}, —CN, C(Q²)R^{c1}, —C(Q²)OR^{d1}, —C(Q²)N(R^{e1})R^{f1}, —N₃, —NO₂, —N(R^{g1})R^{h1}, —N(Rⁱ¹)C(Q²)R^{j1}, —N(R^{k1})C(Q²)N(R^{l1})R^{m1}, —N(Rⁿ¹)C(Q²)OR^{o1}, —N(R^{p1})S(O)ₙR^{g1}, —N(R^{r1})S(O)ₙN(R^{s1})R^{t1}, —OR^{u1}, —OC(Q²)R^{v1}, —OC(Q²)N(R^{w1})R^{x1}, —OC(Q²)OR^{y1}, —OS(O)ₙR^{z1}, —SR^{aa1}, —S(O)ₙR^{ab1} or —S(O)ₙN(R^{ac1})R^{ad1};
M represents a cation selected from (R^{M})₄N⁺, Li⁺, Na⁺, K⁺, Rb⁺ or Cs⁺;
each R^{M} independently represents C₁₋₁₂ alkyl optionally substituted by one or more D⁴;
each Q² independently represents ═NR^{ae1}, ═N(OR^{af1}), ═O or ═S;
each R^{b1}, R^{o1}, R^{q1}, R^{y1}, R^{z1} and R^{ab1} independently represents C₁₋₄ alkyl optionally substituted by one or more groups independently selected from D⁴;
each R^{a1}, R^{c1}, R^{d1}, R^{e1}, R^{f1}, R^{g1}, R^{h1}, Rⁱ¹, R^{j1}, R^{k1}, R^{l1}, R^{m1}, Rⁿ¹, R^{p1}, R^{r1}, R^{s1}, R^{t1}, R^{u1}, R^{v1}, R^{w1}, R^{x1}, R^{aa1}, R^{ac1}, R^{ad1}, R^{ae1} and R^{af1} independently represents H or C₁₋₄ alkyl optionally substituted by one or more groups independently selected from D⁴; or
R^{e1} and R^{f1}, R^{g1} and R^{h1}, R^{l1} and R^{m1}, R^{s1} and R^{t1}, R^{w1} and R^{x1} and/or R^{ac1} and R^{ad1} are linked together to form, along with the nitrogen atom to which they are attached, a 3- to 6-membered ring, which ring optionally contains one further heteroatom and which ring optionally is substituted by one or more groups independently selected from F, one or more C₁₋₃ alkyl each optionally and independently substituted by one or more F, and ═O; or
two R^{a1} are linked together to form, along with the boron, and the oxygen atoms to which they are attached, a 5- to 8-membered heterocyclic ring, which ring optionally contains one or more further heteroatoms and which ring optionally is substituted by one or more groups independently selected from halo, C₁₋₃ alkyl optionally substituted by one or more halo, and ═O;
each D⁴ independently represents halo, —OH or —OC₁₋₆ alkyl optionally substituted by one or more halo;
each n independently represents 1 or 2.

2.-5. (canceled)

6. A method as claimed in claim 1 wherein:
R⁵ and R⁶ are independently selected from halo and R^{c}.

7. (canceled)

8. (canceled)

9. A method as claimed in claim 1 wherein:
A represents -L¹-L²-L³-A¹;
each one of L¹ and L³ independently represents a single bond or C₁₋₃alkylene; and
L² represents a single bond, —C(O)— or —S(O)₂—;
A¹ represents phenyl optionally substituted by one to three groups independently selected from Y¹ or heteroaryl optionally substituted by one to three groups independently selected from Y²;
each Y¹ and Y² independently represents halo, R^{b1}, —CN, —C(Q²)R^{c1}, —C(O)OR^{d1}, —C(O)N(R^{e1})R^{f1}, —N(Rⁱ¹)C(O)R^{j1}, —N(R^{p1})S(O)₂R^{q1}, —OR^{u1}, —OC(O)R^{v1}, —S(O)₂R^{ab1} or heteroaryl optionally substituted by one or more groups independently selected from Z³;
each Z³ independently represents halo or C₁₋₃ alkyl optionally substituted by one or more F;
Q² represents ═O or ═N(OH);
each R^{b1} independently represents F, —OH or —OMe;
each R^{q1} and R^{ab1} independently represents C₁₋₃ alkyl optionally substituted by one or more F; and
each R^{c1}, R^{d1}; R^{e1}; R^{f1}, Rⁱ¹, R^{j1}; R^{p1}; R^{r1}; R^{u1} or R^{v1} independently represents H or C₁₋₃alkyl optionally substituted by one or more F.

10. A method as claimed in claim 9 wherein:
A represents -L¹-L²-L³-A¹;
-L¹-L²-L³- represents a single bond, —CH₂—, —CH₂CH₂—, —CH(Me)-, —C(O)— or —S(O)₂—; and
A¹ represents:
(i) phenyl optionally substituted by one, two or three groups independently selected from F, Cl, bromo, —CN, —CH(OH)CH═CH₂, —C(═NOH)H, —C(O)H, —C(O)NH₂, —C(O)OH, —C(O)OMe, —NH₂, —N(H)C(O)Me, —N(H)C(O)CH═CH₂, —OH, —OMe, —OCF₃, —OC(O)Me, —S(O)₂Me, pyridinyl, thiazolyl and 1,2,4-triazol-1-yl; or
(ii) heteroaryl optionally substituted by halo or C₁₋₃ alkyl optionally substituted by one or more F.

11. A method as claimed in claim 1 wherein:
A represents -L¹-L²-L³-A¹;
each one of L¹ and L³ independently represents a single bond or C₁₋₃alkylene; and
L² represents a single bond, —C(O)— or —S(O)₂—;
A¹ represents:
(i) phenyl substituted by —BF₃K or —B(OR^{a1})₂, and optionally substituted by one or more groups independently selected from F, Cl, methyl, difluoromethyl, trifluoromethyl, —OH and —OMe;
(ii) monocyclic heteroaryl substituted by —BF₃K or —B(OR^{a1})₂, and optionally substituted by one or more groups independently selected from F, Cl, methyl, difluoromethyl, trifluoromethyl, —OH and —OMe; or
(iii) bicyclic, boron containing, partly aromatic heteroaryl substituted on the boron by —OH and optionally substituted by one or more groups independently selected from F, Cl, methyl, difluoromethyl, trifluoromethyl, —OH and —OMe;
each R^{a1} independently represents H or C₁₋₃alkyl optionally substituted by one or more F; or
two R^{a1} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O.

12. A method as claimed in claim 11 wherein:
A represents -L¹-L²-L³-A¹;
-L¹-L²-L³- represents a single bond, —CH₂—, —CH₂CH₂—, —CH(Me)-, —C(O)— or —S(O)₂—; and
A¹ represents phenyl substituted by —B(OR^{a1})₂ and optionally and independently substituted by one or two groups independently selected from F, Cl, methyl, —OH or —OMe.

13. A method as claimed in claim 12 wherein:
A represents —CH₂-A¹; and
A¹ represents phenyl substituted by —B(OH)₂, —B(OMe)₂, 4,4,5,5-tetramethyl-1,3,2-dioxaborolan-2-yl or 6-methyl-1,3,6,2-dioxazaborocane-4,8-dion-2-yl.

14. (canceled)

15. (canceled)

16. A method as claimed in claim 1 wherein:
R^{a} represents:
(i) C₁₋₃alkyl optionally substituted by one to three F and —OC₁₋₃alkyl optionally substituted by one to three F;
(ii) C₁₋₃alkylphenyl optionally substituted by one or two groups independently selected from F, Cl, methyl, difluoromethyl, trifluoromethyl, methoxy, difluoromethoxy and trifluoromethoxy; or
(iii) phenyl optionally substituted by one or two groups independently selected from F, Cl, methyl, difluoromethyl, trifluoromethyl, methoxy, difluoromethoxy and trifluoromethoxy; and
R^{b} represents C₁₋₂alkyl optionally substituted by one or more F.

17. A method as claimed in claim 1 wherein:
R² represents methyl, isopropyl, cyclopropyl, trifluoromethyl, methoxymethyl, benzyl, phenyl or methoxy.

18. (canceled)

19. (canceled)

20. A method as claimed in claim 1, wherein the compound is selected from:
1-(4-methoxybenzyl)-2-methyl-4-nitro-1H-benzo[d]imidazole,
1-(4-methoxybenzyl)-4-nitro-2-phenyl-1H-benzo[d]imidazole,
2-benzyl-1-(4-methoxybenzyl)-4-nitro-1H-benzo[d]imidazole,
1-(4-methoxybenzyl)-4-nitro-2-(trifluoromethyl)-1H-benzo[d]imidazole,
2-methoxy-1-(4-methoxybenzyl)-4-nitro-1H-benzo[d]imidazole,
1-(4-methoxybenzyl)-2-(methoxymethyl)-4-nitro-1H-benzo[d]imidazole,
2-isopropyl-1-(4-methoxybenzyl)-4-nitro-1H-benzo[d]imidazole,
(4-((4-nitro-2-(trifluoromethyl)-1H-benzo[d]imidazol-1-yl)methyl)phenyl)boronic acid,
5,6-difluoro-1-(4-methoxybenzyl)-2-methyl-4-nitro-1H-benzo[d]imidazole,
1-benzyl-5,6-difluoro-2-methyl-4-nitro-1H-benzo[d]imidazole,
4-((5,6-difluoro-2-methyl-4-nitro-1H-benzo[d]imidazol-1-yl)methyl)benzonitrile,
5,6-difluoro-1-(4-fluorobenzyl)-2-methyl-4-nitro-1H-benzo[d]imidazole,
1-(4-(1H-1,2,4-triazol-1-yl)benzyl)-5,6-difluoro-2-methyl-4-nitro-1H-benzo[d]imidazole,
5,6-difluoro-2-methyl-4-nitro-1-(1-phenylethyl)-1H-benzo[d]imidazole,
(4-((5,6-difluoro-2-methyl-4-nitro-1H-benzo[d]imidazol-1-yl)methyl)phenyl)boronic acid,
5,6-dichloro-2-methyl-4-nitro-1-phenyl-1H-benzo[d]imidazole,
5,6-dichloro-2-methyl-4-nitro-1-(phenylsulfonyl)-1H-benzo[d]imidazole,
(5,6-dichloro-2-methyl-4-nitro-1H-benzo[d]imidazol-1-yl)(phenyl)methanone,
1-benzyl-5,6-dichloro-2-methyl-4-nitro-1H-benzo[d]imidazole,
5,6-dichloro-1-(4-methoxybenzyl)-2-methyl-4-nitro-1H-benzo[d]imidazole,
5,6-dichloro-1-(3-methoxybenzyl)-2-methyl-4-nitro-1H-benzo[d]imidazole,
4-((5,6-dichloro-2-methyl-4-nitro-1H-benzo[d]imidazol-1-yl)methyl)phenyl acetate,
N-(4-((5,6-dichloro-2-methyl-4-nitro-1H-benzo[d]imidazol-1-yl)methyl)phenyl)acetamide,
4-((5,6-dichloro-2-methyl-4-nitro-1H-benzo[d]imidazol-1-yl)methyl)aniline,
4-((5,6-dichloro-2-methyl-4-nitro-1H-benzo[d]imidazol-1-yl)methyl)phenol,
5,6-dichloro-2-methyl-1-(4-methylbenzyl)-4-nitro-1H-benzo[d]imidazole,
4-((5,6-dichloro-2-methyl-4-nitro-1H-benzo[d]imidazol-1-yl)methyl)benzaldehyde,
4-((5,6-dichloro-2-methyl-4-nitro-1H-benzo[d]imidazol-1-yl)methyl)benzaldehyde oxime,
1-(4-((5,6-dichloro-2-methyl-4-nitro-1H-benzo[d]imidazol-1-yl)methyl)phenyl)prop-2-en-1-ol,
1-(4-((5,6-dichloro-2-methyl-4-nitro-1H-benzo[d]imidazol-1-yl)methyl)phenyl)prop-2-en-1-one,
2-bromo-5-((5,6-dichloro-2-methyl-4-nitro-1H-benzo[d]imidazol-1-yl)methyl)-benzaldehyde,
N-(4-((5,6-dichloro-2-methyl-4-nitro-1H-benzo[d]imidazol-1-yl)methyl)phenyl)acrylamide,
5,6-dichloro-1-(2-(3,5-dimethyl-1H-pyrazol-4-yl)ethyl)-2-methyl-4-nitro-1H-benzo[d]imidazole,
1-(4-(1H-pyrazol-1-yl)benzyl)-5,6-dichloro-2-methyl-4-nitro-1H-benzo[d]imidazole,
1-(4-(1H-1,2,4-triazol-1-yl)benzyl)-5,6-dichloro-2-methyl-4-nitro-1H-benzo[d]imidazole,
1-(4-(1H-pyrrol-1-yl)benzyl)-5,6-dichloro-2-methyl-4-nitro-1H-benzo[d]imidazole,
4-((5,6-dichloro-2-methyl-4-nitro-1H-benzo[d]imidazol-1-yl)methyl)benzoic acid,
methyl 4-((5,6-dichloro-2-methyl-4-nitro-1H-benzo[d]imidazol-1-yl)methyl)benzoate,
4-((5,6-dichloro-2-methyl-4-nitro-1H-benzo[d]imidazol-1-yl)methyl)benzamide,
4-((5,6-dichloro-2-methyl-4-nitro-1H-benzo[d]imidazol-1-yl)methyl)benzonitrile,
5,6-dichloro-2-methyl-1-(4-(methylsulfonyl)benzyl)-4-nitro-1H-benzo[d]imidazole,
4-((5,6-dichloro-2-methyl-4-nitro-1H-benzo[d]imidazol-1-yl)methyl)-2-methylthiazole,
5,6-dichloro-1-((6-chloropyridin-3-yl)methyl)-2-methyl-4-nitro-1H-benzo[d]imidazole,
(4-((5,6-dichloro-2-methyl-4-nitro-1H-benzo[d]imidazol-1-yl)methyl)phenyl)boronic acid,
(4-((5,6-dichloro-2-cyclopropyl-4-nitro-1H-benzo[d]imidazol-1-yl)methyl)phenyl)boronic acid,
(2-((5,6-dichloro-2-methyl-4-nitro-1H-benzo[d]imidazol-1-yl)methyl)phenyl)boronic acid,
(3-((5,6-dichloro-2-methyl-4-nitro-1H-benzo[d]imidazol-1-yl)methyl)phenyl)boronic acid,
5,6-dichloro-2-methyl-4-nitro-1-(4-(trifluoro-l4-boranyl)benzyl)-1H-benzo[d]imidazole, or potassium salt thereof,
5,6-dichloro-2-methyl-4-nitro-1-(4-(4,4,5,5-tetramethyl-1,3,2-dioxaborolan-2-yl)benzyl)-1H-benzo[d]imidazole,
2-(4-((5,6-dichloro-2-methyl-4-nitro-1H-benzo[d]imidazol-1-yl)methyl)phenyl)-6-methyl-1,3,6,2-dioxazaborocane-4,8-dione,
(4-(2-(5,6-dichloro-2-methyl-4-nitro-1H-benzo[d]imidazol-1-yl)ethyl)phenyl)boronic acid,
2,5,6-trim ethyl-4-nitro-1-(3,4,5-trim ethoxybenzyl)-1H-benzo[d]imidazole,
1-((6-chlorobenzo[d][1,3]dioxol-5-yl)methyl)-2,5,6-trimethyl-4-nitro-1H-benzo[d]imidazole,
1-(4-methoxybenzyl)-2,5,6-trim ethyl-4-nitro-1H-benzo[d]imidazole,
1-(4-chlorobenzyl)-2,5,6-trimethyl-4-nitro-1H-benzo[d]imidazole,
1-(4-fluorobenzyl)-2,5,6-trim ethyl-4-nitro-1H-benzo[d]imidazole,
2,5,6-trim ethyl-4-nitro-1-(4-(trifluoromethoxy)benzyl)-1H-benzo[d]imidazole,
1-(4-methoxybenzyl)-5,6-dimethyl-4-nitro-2-(trifluoromethyl)-1H-benzo[d]imidazole,
2,5,6-trim ethyl-4-nitro-1-(1-phenylethyl)-1H-benzo[d]imidazole,
(4-((2,5,6-trimethyl-4-nitro-1H-benzo[d]imidazol-1-yl)methyl)phenyl)boronic acid,
(4-((5,6-dimethyl-4-nitro-2-(trifluoromethyl)-1H-benzo[d]imidazol-1-yl)methyl)-phenyl)boronic acid,
1-(4-methoxybenzyl)-4-nitro-1H-benzo[d]imidazole,
(4-((4-nitro-1H-benzo[d]imidazol-1-yl)methyl)phenyl)boronic acid,
2-benzyl-1-(4-methoxybenzyl)-7-nitro-1H-benzo[d]imidazole,
2-methoxy-1-(4-methoxybenzyl)-7-nitro-1H-benzo[d]imidazole,
1-(4-methoxybenzyl)-7-nitro-2-phenyl-1H-benzo[d]imidazole, and
(4-((5,6-difluoro-2-methyl-7-nitro-1H-benzo[d]imidazol-1-yl)methyl)phenyl)boronic, acid,
or is a pharmaceutically acceptable salt thereof.

21. A compound of formula I, 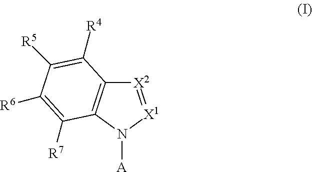
or a pharmaceutically acceptable salt thereof, wherein:
A represents -L¹-L²-L³-A¹;
A¹ represents:
(i) aryl substituted by —BF₃M or —B(OR^{a1})₂, and optionally substituted by one or more groups independently selected from Y¹;
(ii) heteroaryl substituted by —BF₃M or —B(OR^{a1})₂, and optionally substituted by one or more groups independently selected from Y²; or
(iii) bicyclic, boron containing, partly aromatic heteroaryl substituted on the boron by —OH and optionally substituted by one or more groups independently selected from Y³;
each one of L¹ and L³ independently represents a single bond or C₁₋₃ alkylene optionally substituted by one or more halo;
L² represents a single bond, —C(Q)-, —N(R¹)—, —O—, —S(O)ₙ—, —C(Q)N(R¹)—, —N(R¹)C(Q)-, —C(O)O—, —OC(O)—, —S(O)ₙN(R¹)— or —N(R¹)S(O)ₙ—;
X¹ represents C(R²);
X² represents N;
R¹ represents H or C₁₋₆ alkyl optionally substituted by one or more halo;
R² represents H, R^{a} or —OR^{b};
R⁴ and R⁷ independently represent H, halo, —CN, R^{c}, —C(H)(CF₃)OH, —C(CF₃)₂OH, —C(OH)₂CF₃, —N₃, —NO₂, —N(R^{d})R^{e}, —N(R^{f})C(Q¹)R^{g}, —N(R^{h})S(O)ₙRⁱ, —OR^{j}, —SR^{k} or —C(O)R⁸;
R⁵ and R⁶ independently represent H, halo, —CN, R^{c}, —N₃, —NO₂, —OR^{j} or —SR^{k}; or
R⁴ and R⁵, R⁵ and R⁶ and/or R⁶ and R⁷ are linked together to form, along with the carbon atoms to which they are attached, a 5- or 6-membered ring, which ring optionally contains one to three heteroatoms and/or one or two further double bonds, and which ring optionally is substituted by one or more groups independently selected from halo, —OR^{j}, C₁₋₃ alkyl optionally substituted by one or more halo, and Q¹;
each R⁸ independently represents —OR^{l}, —N(H)R^{m}, —N(H)C(Q¹)Rⁿ, —N(H)C(Q¹)N(R^{o})R^{p}, —N(H)OH or —N(H)S(O)ₙR^{q};
Q represents ═O or ═S;
Q¹ represents ═O, ═NR^{r} or ═S;
R^{a} represents C₁₋₆ alkyl optionally substituted by one or more groups independently selected from D¹, aryl optionally substituted by one or more groups independently selected from D² or heteroaryl optionally substituted by one or more groups independently selected from D³;
each R^{c} and R^{q} independently represents C₁₋₆ alkyl optionally substituted by one or more halo;
each R^{b}, R^{d}, R^{e}, R^{f}, R^{g}, R^{h}, Rⁱ, R^{j}, R^{k}, R^{l}, R^{m}, Rⁿ, R^{o}, R^{p} and R^{r} independently represents H or C₁₋₆alkyl optionally substituted by one or more halo; or
R^{d} and R^{e} and/or R^{o} and R^{p} are linked together to form, along with the nitrogen atom to which they are attached, a 3- to 6-membered ring, which ring optionally contains one further heteroatom and which ring optionally is substituted by one or more halo, one or more C₁₋₃alkyl each optionally and independently substituted by one or more F, or ═O;
D¹ represents halo, —OC₁₋₆ alkyl optionally substituted by one or more halo, aryl optionally substituted by one or more groups independently selected from D² or heteroaryl optionally substituted by one or more groups independently selected from D³;
each D² and D³ independently represents halo, C₁₋₆alkyl optionally substituted by one or more halo or —OC₁₋₆ alkyl optionally substituted by one or more halo;
each Y¹, Y² and Y³ independently represents halo, R^{b1}, —CN, or —OR^{u1};
M represents a cation selected from (R^{M})₄N⁺, Li⁺, Na⁺, K⁺, Rb⁺ or Cs⁺;
each R^{M} independently represents C₁₋₁₂alkyl optionally substituted by one or more D⁴;
each R^{b1} independently represents C₁₋₆ alkyl optionally substituted by one or more groups independently selected from D⁴;
each R^{a1} and R^{u1} independently represents H or C₁₋₆ alkyl optionally substituted by one or more groups independently selected from D⁴; or
two R^{a1} are linked together to form, along with the boron, and the oxygen atoms to which they are attached, a 5- to 8-membered heterocyclic ring, which ring optionally contains one or more further heteroatoms and which ring optionally and independently is substituted by one or more groups independently selected from halo, C₁₋₃ alkyl optionally substituted by one or more halo, and ═O;
each D⁴ independently represents halo, —OH or —OC₁₋₆ alkyl optionally substituted by one or more halo;
each n independently represents 1 or 2;
provided that at least one of R⁴ and R⁷ represent —C(H)(CF₃)OH, —C(CF₃)₂OH, —C(OH)₂CF₃, —NO₂ or —C(O)R⁸; and
provided that formula I does not represent
1-(4-boronobenzyl)-2-ethoxy-1H-benzo[d]imidazole-7-carboxylic acid,
ethyl 1-(4-(5,5-dim ethyl-1, 3,2-dioxaborinan-2-yl)benzyl)-2-ethoxy-1H-benzo[d]imidazole-7-carboxylate,
methyl 1-(3-(4,4,5,5-tetramethyl-1,3,2-dioxaborolan-2-yl)benzyl)-1H-indole-7-carboxylate, or
methyl 1-(4-(4,4,5,5-tetramethyl-1, 3,2-dioxaborolan-2-yl)benzyl)-1H-indole-7-carboxylate.

22. A compound as claimed in claim 21 wherein:
each one of L¹ and L³ independently represents a single bond or C₁₋₃alkylene;
L² represents a single bond, —C(O)—, —S(O)₂— or —C(O)N(H)—;
A¹ represents:
(i) phenyl substituted by —BF₃K or —B(OR^{a1})₂, and optionally substituted by F, Cl, methyl, difluoromethyl, trifluoromethyl, —OH or —OMe;
(ii) monocyclic heteroaryl substituted by —BF₃K or —B(OR^{a1})₂, and optionally substituted by F, Cl, methyl, difluoromethyl, trifluoromethyl, —OH or —OMe; or
(iii) bicyclic, boron containing, partly aromatic heteroaryl substituted on the boron by —OH and optionally substituted by one or more groups independently selected from F, Cl, methyl, difluoromethyl, trifluoromethyl, —OH and —OMe;
R² represents R^{a} or —OR^{b};
R⁴ represents —NO₂ or —C(O)R⁸;
R⁵ and R⁶ independently represent H, halo or R^{c};
R⁷ represents H; or
R⁵ and R⁶ or R⁶ and R⁷ are linked together to form, along with the carbon atoms to which they are attached, a 5- or 6-membered ring, which ring optionally contains one to three heteroatoms and/or one or two further double bonds, and which ring optionally is substituted by one or more groups independently selected from F or R^{c};
R⁸ represents —OR^{l}, —N(H)R^{m}, —N(H)C(O)Rⁿ, —N(H)C(O)N(R^{o}R^{p}, —N(H)OH and —N(H)S(O)₂R^{q};
R^{a} represents:
(i) C₁₋₃ alkyl optionally substituted by one to three groups independently selected from F and —OC₁₋₃alkyl optionally substituted by one to three F;
(ii) C₁₋₃alkylphenyl optionally substituted by one or two groups independently selected from F, Cl, methyl, difluoromethyl, trifluoromethyl, methoxy, difluoromethoxy and trifluoromethoxy; or
(iii) phenyl optionally substituted by one or two groups independently selected from F, Cl, methyl, difluoromethyl, trifluoromethyl, methoxy, difluoromethoxy and trifluoromethoxy;
R^{b} represents C₁₋₂ alkyl optionally substituted by one or more fluoroF;
each R^{c} and R^{q} independently represents C₁₋₆alkyl optionally substituted by one or more F;
each R^{d}, R^{e}, R^{f}, R^{g}, R^{h}, Rⁱ, R^{j}, R^{k}, R^{l}, R^{m}, Rⁿ and R^{o} independently represents H or C₁₋₆alkyl optionally substituted by one or more F; or
R^{d} and R^{e} and/or R^{o} and R^{p} are linked together to form, along with the nitrogen atom to which they are attached, a 3- to 6-membered ring, which ring optionally contains one further heteroatom and which ring optionally is substituted by one or more groups independently selected from F, C₁₋₃alkyl optionally substituted by one or more F, and ═O;
each R^{a1} independently represents H or C₁₋₃alkyl optionally substituted by one or more F; or
two R^{a1} are linked together to form, along with the boron, and the oxygen atoms to which they are attached, a 5-, 6- or 8-membered heterocyclic ring, which ring optionally contains one or two further heteroatoms and which ring optionally is substituted by one or more C₁₋₃alkyl and/or one or more ═O.

23. A compound as claimed in claim 22 wherein:
-L¹-L²-L³- represents a single bond, —CH₂—, —CH₂CH₂—, —CH(Me)-, —C(O)— or —S(O)₂—;
A¹ represents phenyl substituted by —BF₃K or —B(OR^{a1})₂, and optionally substituted by F, Cl, methyl, —OH or —OMe.

24. A compound as claimed in claim 23 wherein:
A represents —CH₂-A¹;
A¹ represents phenyl substituted by —B(OH)₂, —B(OMe)₂, 4,4,5,5-tetramethyl-1,3,2-dioxaborolan-2-yl or 6-methyl-1,3,6,2-dioxazaborocane-4,8-dion-2-yl;
R² represents methyl, isopropyl, cyclopropyl, trifluoromethyl, methoxymethyl, benzyl, phenyl or methoxy;
R⁴ represents —NO₂;
R⁵ and R⁶ independently represent H, halo or R^{c}; and
R⁷ represents H.

25. (canceled)

26. A compound as claimed in claim 24 wherein:
R⁵ and R⁶ are independently selected from F, Cl, and methyl.

27.-35. (canceled)

36. A compound as claimed in claim 21, selected from
(4-((4-nitro-2-(trifluoromethyl)-1H-benzo[d]imidazol-1-yl)methyl)phenyl)boronic acid,
(4-((5,6-difluoro-2-methyl-4-nitro-1H-benzo[d]imidazol-1-yl)methyl)phenyl)boronic acid,
(4-((5,6-dichloro-2-methyl-4-nitro-1H-benzo[d]imidazol-1-yl)methyl)phenyl)boronic acid,
(4-((5,6-dichloro-2-cyclopropyl-4-nitro-1H-benzo[d]imidazol-1-yl)methyl)phenyl)boronic acid,
(2-((5,6-dichloro-2-methyl-4-nitro-1H-benzo[d]imidazol-1-yl)methyl)phenyl)boronic acid,
(3-((5,6-dichloro-2-methyl-4-nitro-1H-benzo[d]imidazol-1-yl)methyl)phenyl)boronic acid,
5,6-dichloro-2-methyl-4-nitro-1-(4-(trifluoro-l4-boranyl)benzyl)-1H-benzo[d]imidazole, or potassium salt thereof,
5,6-dichloro-2-methyl-4-nitro-1-(4-(4,4,5,5-tetramethyl-1, 3,2-dioxaborolan-2-yl)benzyl)-1H-benzo[d]imidazole,
2-(4-((5,6-dichloro-2-methyl-4-nitro-1H-benzo[d]imidazol-1-yl)methyl)phenyl)-6-methyl-1,3,6,2-dioxazaborocane-4,8-dione,
(4-(2-(5,6-dichloro-2-methyl-4-nitro-1H-benzo[d]imidazol-1-yl)ethyl)phenyl)boronic acid,
(4-((2,5,6-trimethyl-4-nitro-1H-benzo[d]imidazol-1-yl)methyl)phenyl)boronic acid, or
(4-((5,6-dimethyl-4-nitro-2-(trifluoromethyl)-1H-benzo[d]imidazol-1-yl)methyl)phenyl)-boronic acid,
or a pharmaceutically acceptable salt thereof.

37.-39. (canceled)

40. The method according to claim 1, wherein the proliferative disorder is cancer.

41. The method according to claim 1, wherein the proliferative disorder is inflammation.

42. A pharmaceutical formulation comprising a compound as claimed in claim 21, or a pharmaceutically acceptable salt thereof, in admixture with a pharmaceutically acceptable adjuvant, diluent or carrier.

43. (canceled)

44. (canceled)

45. The method as claimed in claim 40, wherein the cancer is selected from the group comprising Soft Tissue Cancers: sarcoma (angiosarcoma, fibrosarcoma, rhabdomyosarcoma, liposarcoma), myxoma, rhabdomyoma, fibroma, lipoma and teratoma; Lung cancers/disorders: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal cancers/disorders: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract cancers/disorders: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver cancers/disorders: hepatoma (hepatocellular carcinoma), cholangiocarcinoma, hepatoblastoma, angiosarcoma, hepatocellular adenoma, hemangioma; Bone cancers/disorders: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system cancers/disorders: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological cancers/disorders: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic cancers/disorders: blood and bone marrow (myeloid leukemia [acute and chronic], acute lymphoblastic leukemia, chronic lymphocytic leukemia, myeloproliferative disorders, multiple myeloma, myelodysplastic syndrome), Hodgkin's disorder, non-Hodgkin's lymphoma [malignant lymphoma]; Skin cancers/disorders: malignant melanoma, basal cell carcinoma, squamous cell carcinoma, Kaposi's sarcoma, moles dysplastic nevi, lipoma, angioma, dermatofibroma, keloids; Adrenal glands cancers/disorders, neuroblastoma, neurofibromatosis and head and neck cancers.

46. The method as claimed in claim 45, wherein the cancer is selected from the group comprising acute myeloid leukaemia, acute lymphocytic leukaemia, myelodysplastic sindrome, chronic myelomonocytic leukaemia, lymphoma, advanced stomach cancer, oesophageal cancer or ovarian cancer.

47. The method as claimed in claim 41, wherein the inflammation is selected from the group comprising allergic disorders, asthma, childhood wheezing, chronic obstructive pulmonary disorder, bronchopulmonary dysplasia, cystic fibrosis, interstitial lung disorder (e.g. sarcoidosis, pulmonary fibrosis, scleroderma lung disorder, and usual interstitial in pneumonia), ear nose and throat disorders (e.g. rhinitis, nasal polyposis, and otitis media), eye disorders (e.g. conjunctivitis and giant papillary conjunctivitis), skin disorders (e.g. psoriasis, dermatitis, and eczema), rheumatic disorders (e.g. rheumatoid arthritis, arthrosis, psoriasis arthritis, osteoarthritis, systemic lupus erythematosus, systemic sclerosis), vasculitis (e.g. Henoch-Schonlein purpura, Löffler's syndrome and Kawasaki disorder), cardiovascular disorders (e.g. atherosclerosis), gastrointestinal disorders (e.g. eosinophilic disorders in the gastrointestinal system, inflammatory bowel disorder, irritable bowel syndrome, colitis, celiaci and gastric haemorrhagia), urologic disorders (e.g. glomerulonephritis, interstitial cystitis, nephritis, nephropathy, nephrotic syndrome, hepatorenal syndrome, and nephrotoxicity), disorders of the central nervous system (e.g. cerebral ischemia, spinal cord injury, migraine, multiple sclerosis, and sleep-disordered breathing), endocrine disorders (e.g. autoimmune thyreoiditis, diabetes-related inflammation), urticaria, anaphylaxis, angioedema, oedema in Kwashiorkor, dysmenorrhoea, burn-induced oxidative injury, multiple trauma, pain, toxic oil syndrome, endotoxin chock, sepsis, bacterial infections (e.g. from Helicobacter pylori, Pseudomonas aerugiosa or Shigella dysenteriae), fungal infections (e.g. vulvovaginal candidasis), viral infections (e.g. hepatitis, meningitis, parainfluenza and respiratory syncytial virus), sickle cell anemia and hypereosinofilic syndrome.

48. (canceled)

49. A combination product comprising:
(A) a compound or a pharmaceutically acceptable salt thereof as claimed in claim 21; and
(B) one or more other therapeutic agent(s),
wherein each one of components (A) and (B) is formulated in admixture with a pharmaceutically-acceptable adjuvant, diluent or carrier.

50. A combination product as claimed in claim 49, wherein component (B) is selected from the group comprising anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors; kinase inhibitors; angiogenesis inhibitors; immunotherapeutic agents; pro-apoptotic agents; and cell cycle signaling inhibitors.

51. A combination product as claimed in claim 49, wherein component (B) is selected from the group comprising cytarabine, fludarabine, cladribine, clofarabine, nelarabine, capecitabine, floxuridine, deoxycoformycin, azacitidine, decitabine, gemcitabine, sapacitabine, zebularine, fluorouracil and 4′-thio-2′-deoxycytidine.

52. A combination product as claimed in claim 49 comprising a compound selected from the group comprising
2-(4-((5,6-dichloro-2-methyl-4-nitro-1H-benzo[d]imidazol-1-yl)methyl)phenyl)-6-methyl-1,3,6,2-dioxazaborocane-4,8-dione,
(4-((5,6-dichloro-2-methyl-4-nitro-1H-benzo[d]imidazol-1-yl)methyl)phenyl)boronic acid,
(4-((5,6-dichloro-2-cyclopropyl-4-nitro-1H-benzo[d]imidazol-1-yl)methyl)phenyl)boronic acid,
5,6-dichloro-2-methyl-4-nitro-1-(4-(4,4,5,5-tetramethyl-1,3,2-dioxaborolan-2-yl)benzyl)-1H-benzo[d]imidazole,
5, 6-dichloro-2-methyl-4-nitro-1-(4-(trifluoro-l4-boranyl)benzyl)-1H-benzo[d]imidazole, potassium salt, and
a therapeutic agent selected from the group comprising azacitidine, decitabine and gemcitabine.

53. A method of treatment of a proliferative disorder, comprising administration of a therapeutically effective amount of a compound according claim 21, to a patient in need of such treatment.

54. The method as claimed in claim 53, wherein the disorder is cancer and/or inflammation.

55. The method as claimed in claim 53, wherein the treatment is combined with radiation therapy.
